(19) United States
(12) Patent Application Publication
(10) Pub. No.: US 2024/0287589 A1
(43) Pub. Date: Aug. 29, 2024

(54) PHOTOREACTIVE AND CLEAVABLE PROBES FOR TAGGING BIOMOLECULES

(71) Applicant: Syncell (Taiwan) Inc., Taipei City (TW)

(72) Inventors: Chih-Wei CHANG, Taipei (TW); Hsiang-Ju KAI, Taipei City (TW); Chia-Wen CHUNG, Taipei (TW); Yi-De CHEN, Taipei (TW); Jung-Chi LIAO, Taipei City (TW)

(21) Appl. No.: 18/611,306

(22) Filed: Mar. 20, 2024

Related U.S. Application Data
(63) Continuation-in-part of application No. PCT/US2022/076672, filed on Sep. 19, 2022.
(60) Provisional application No. 63/246,287, filed on Sep. 20, 2021.

(51) Int. Cl. C12Q 1/6841; G01N 33/58

(52) U.S. Cl. CPC C12Q 1/6841; G01N 33/58

(57) ABSTRACT

Compositions including photoreactive and cleavable probes and methods of using the probes. The probes may include a tag conjugatable to a label, a cleavable linker linkable to a bait molecule, and a light activated warhead, which may be configured to covalently bond an anchoring strand to a probing strand upon application of light energy. The compositions and methods may be useful for analyzing biomolecules, such as identifying proximal molecules in cell or tissue samples.

### CROSS REFERENCE TO RELATED APPLICATIONS

This application claims priority to PCT/US2022/076672, titled “PHOTOREACTIVE AND CLEAVABLE PROBES FOR TAGGING BIOMOLECULES”, filed on Sep. 19, 2022, which claims priority to U.S. Provisional Patent Application No. 63/246,287, titled “PHOTOREACTIVE AND CLEAVABLE PROBES FOR TAGGING BIOMOLECULES”, filed on Sep. 20, 2021, which are herein incorporated by reference in their entirety.

### SEQUENCE LISTING

The instant application contains a Sequence Listing which has been submitted electronically in XML file format and is hereby incorporated by reference in its entirety. Said XML copy, created Dec. 1, 2022, is named 14815-705_600_SL.xml and is 41,455 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and compositions for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples.

### BACKGROUND

Cells are composed of different types of biological molecules (biomolecules). The biomolecules in the cells interact with neighbor biomolecules in the subcellular environment to form complexes, organelles, or other assemblies and to carry out various essential cell functions. Characterizing the subcellular environment, within which biomolecules interact with one another, and how the biomolecules function together is very challenging. Biomolecules are small, and they exist in a cell environment with tens of millions of other molecules. The interactions between neighboring biomolecules are frequently weak, and techniques used to study biomolecules disrupt their interactions. While techniques such as yeast two-hybridization assays and more recently proximity labeling have advanced our understanding of the cell environment, these techniques suffer from various limitations such as nonspecific binding, slow reaction times and disruption of the natural cell environment, resulting in false positives and missed interactions. What is needed are better tools for determining naturally occurring biomolecule interactions. Described herein are systems, compositions, and methods to better analyze endogenous biomolecule interactions.

### SUMMARY OF THE DISCLOSURE

Described herein are systems, compositions, and methods to better analyze endogenous biomolecule interactions. The methods and compositions may be useful for identifying, tagging, and analyzing biomolecules. Specifically described are cleavable probes useful for photoactivated and tagging of subsets of biomolecules. The methods and compositions may be particularly useful for analyzing biological samples, such as identifying proximal biomolecules in cell or tissue samples. These probes may be especially useful for selectively tagging and proximity labeling of biomolecules via selective light illumination through a microscope system. In general, in one embodiment, a photoreactive and cleavable probe, including a nucleic acid anchoring strand, wherein the anchoring strand can include a bait attachment site, a nucleic acid probing strand, wherein the probing strand forms a double-stranded structure with the anchoring strand along a complementary sequence, a cleavable site in the double-stranded structure, wherein the anchoring strand and probing strand are configured to break at the cleavable site in response to application of a cleaver, a light-activated warhead disposed in the probe, wherein the light-activated warhead is configured to covalently bond the anchoring strand to the probing strand upon application of light energy, and a tag bound to the probing strand, wherein the tag is configured to conjugate to a detectable label.

This and other embodiments can include one or more of the following features. The anchoring strand and the probing strand include DNA, RNA, or both DNA and RNA. The anchoring strand is longer than the probing strand. The anchoring strand and probing strand have at least 6 complementary nucleotides in a continuous row and at least 20 complementary nucleotides in total. The double-stranded structure is at least 10 nucleotides in length. A melting temperature Tₘ of the double-stranded structure is from 52° C. to 60° C. A melting temperature Tₘ of the double-stranded structure is at least 50° C. The anchoring strand and the probing strand are at least 10 nucleotides in length, at least 20 nucleotides in length, or at least 30 nucleotides in length. The anchoring strand and the probing strand are no more than 40 nucleotides in length, no more than 50 nucleotides in length, or no more than 60 nucleotides in length. The anchoring strand and the probing strand are between 15 nucleotides in length and 40 nucleotides in length. The anchoring strand can include a primary sequence and the tag is on the same side of the primary sequence as the bait attachment site, relative to the cleavable site. The probe is attached to a bait molecule at the bait attachment site. The probe is covalently attached to the bait molecule. The bait molecule includes an antibody, a CLIP-tag, a HaloTag, protein A, protein G, protein L, an RNA molecule, a small molecule, or a SNAP-tag. The bait molecule includes an antibody. The bait molecule includes a secondary antibody. The tag includes a biotin derivative, a CLIP-tag, a click chemistry tag, digoxigenin, a HaloTag, a peptide tag, or a SNAP-tag. The biotin derivative includes the moiety of

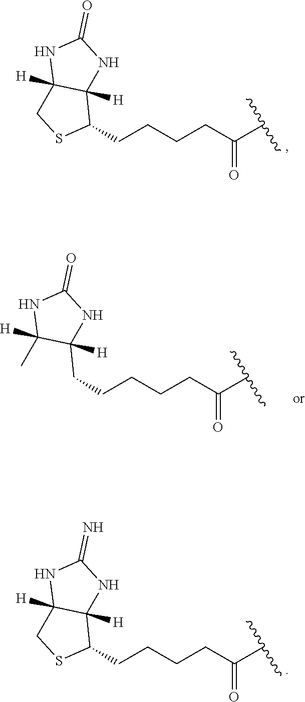

The cleavable site includes an endonuclease site. The cleavable site includes a restriction enzyme site. The light-activated warhead includes a nucleobase. The light-activated warhead includes a thymine-specific warhead. The light-activated warhead includes a nucleobase-specific psoralen, including the moiety of

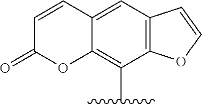 [SMILES: *c1c2occc2cc2ccc(=O)oc12]

or a nucleobase-specific azide. The light-activated warhead includes a nucleobase-specific 3-cyanovinylcarbazole nucleoside (CNVK), including the moiety of

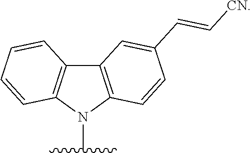 [SMILES: *n1c2ccccc2c2cc(/C=C/C#N)ccc21]

The light-activated warhead includes a nucleobase-specific trioxsalen, including the moiety of

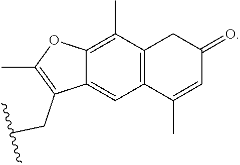 [SMILES: *Cc1c(C)oc2c(C)c3c(cc12)C(C)=CC(=O)C3]

The photoreactive and cleavable probe of any one of the above claims, wherein the light-activated warhead comprises a nucleobase-specific diazirines, including the moiety of

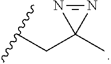 [SMILES: *CC1(C)N=N1]

In general, in one embodiment, a method for photoactivated labeling including conjugating the photoreactive and cleavable probe as above, to a biomolecule in a biological sample, conjugating a bait molecule to a target biomolecule in the biological sample to crosslink the probe and target biomolecule, delivering optical radiation to activate the light-activated warhead and covalently bond the anchoring strand to the probing strand, cleaving the cleavable site in the double-stranded region of the probe to remove a portion of the anchoring strand and the probing strand away from the rest of the probe, and removing the cleaved and unbound probe from the biological sample.

In general, in one embodiment, an analytical method including delivering a photoreactive and cleavable probe to a biological sample, the probe including a nucleic acid anchoring strand and a nucleic acid probing strand, wherein the probing strand forms a double-stranded structure with the anchoring strand along a complementary sequence, selectively illuminating a first region of the biological sample to thereby activate a light-activated warhead disposed in the probe and covalently bond the probing strand to the anchoring strand in the first region, and not illuminating a second region of the biological sample such that the probing strand and anchoring strand are not covalently bonded in the second region, cleaving a cleavable site in the double-stranded region of the probe in the first and second regions to remove a cleaved portion of the anchoring strand and a cleaved portion of the probing strand away from the rest of the probe, delivering a competing nucleic acid strand to the first and second regions, wherein the competing nucleic acid strand is configured to compete with the probing strand for binding to the anchoring strand, and replacing the probing strand with the competing nucleic acid strand in probes in the second region, but not in the first region, wherein the covalent bonding between the probing strand and the anchoring strand in the first region prevents the competing strand from replacing the probing strand in the first region.

This and other embodiments can include one or more of the following features. The probe can further include a tag bound to the probing strand, wherein the tag is configured to conjugate to a detectable label. The step of conjugating a detectable label with the tag and detectably proximity labeling neighbors proximal the target biomolecule by detectable label activity. The step of detectably proximity labeling can include photoselectively proximity labeling a region less than 300 nm, less than 200 nm, or less than 100 nm in diameter or in a longest dimension. A melting temperature Tₘ of the double-stranded structure prior to cleaving the cleavable site can be at least 50° C. A melting temperature Tₘ of the double-stranded structure prior to cleaving the cleavable site can be from 52° C. to 60° C. The method can further include wherein (i) the probe can include a double-stranded structure after the cleaving step, (ii) the probe can include a double-stranded structure after the replacing step, and (iii) a melting temperature Tₘ of the double-stranded structure in the second region after cleaving the cleavable site is lower than a melting temperature Tₘ of the double-stranded structure in the second region after the replacing step in the second region. The melting temperature Tₘ of the double-stranded structure in the second region after cleaving the cleavable site is from 26° C. to 34° C. The melting temperature Tₘ of the double-stranded structure in the second region after the replacing step in the second region is from 44° C. to 53° C. The replacing step can be performed at a temperature higher than the melting temperature Tₘ of the double-stranded structure in the second region after cleaving the cleavable site and lower than the melting temperature Tₘ of the double-stranded structure in the second region after the replacing step. A primary sequence of the anchoring strand, a primary sequence of the probing strand, and/or a primary sequence of the competing strand are bioorthogonal with naturally occurring nucleic acid chains in the biological sample, such that there is no sequence matches more than 10 nucleotides between endogenous and the probing strands or the anchoring strands. The step of cleaving a cleavable site can include cutting the cleavable site with a restriction endonuclease and/or a restriction enzyme. The step of selectively illuminating to activate a light-activated warhead can include activating a nucleobase warhead. The step of selectively illuminating to activate a light-activated warhead can include activating a thymine-specific warhead. The step of selectively illuminating the biological sample can include illuminating from an imaging lighting source of an image-guided microscope system, the method further including imaging the illuminated sample with a controllable camera, acquiring with the camera at least one image of subcellular morphology of the biological sample in a first field of view, processing the at least one image and determining a region of interest in the sample based on the processed image, and obtaining coordinate information of the region of interest. The step of removing cleaved and unbound probe from the first and second regions. The step of selectively illuminating can include illuminating a region for 25 μs/pixel to 400 μs/pixel, for 50 μs/pixel to 300 μs/pixel, or for 75 μs/pixel to 200 μs/pixel. The step of selectively illuminating can include illuminating with a power intensity of from 100 mW to 300 mW. The detectable label can include a catalytic label. The biological sample can include at least one, at least 100, at least 1000 or at least 10,000 live or fixed cells. The biological sample can include fixed cells, tissues, cell extracts, or tissue extracts. The step of selectively illuminating can include illuminating a zone defined by point spread function. The biological sample is disposed on a microscope stage, the method further including removing at least a portion of the illuminated region of the biological sample from the stage. The step of subjecting the sample to mass spectrometry analysis or sequencing analysis. The tag can include a biotin derivative, a CLIP-tag, a click chemistry tag, digoxigenin, a HaloTag, a peptide tag, or a SNAP-tag.

In general, in one embodiment, a kit for labeling biomolecules including the photoreactive and cleavable probe of any of the above in a first container, and an instructional material.

This and other embodiments can include one or more of the following features. A competing strand, wherein the competing strand is complementary to the anchoring strand. When the competing strand and anchoring strand form a double-stranded structure, the structure has a melting temperature Tm of from 44° C. to 53° C.

In general, in one embodiment, a kit for probe generation, including a nucleic acid anchoring strand, wherein the anchoring strand can include a bait attachment site, a nucleic acid probing strand, wherein the probing strand forms a double-stranded structure with the anchoring strand along a complementary sequence, a cleavable site in the double-stranded structure, wherein the anchoring strand and probing strand are configured to break at the cleavable site in response to application of a cleaver, a light-activated warhead, wherein the light-activated warhead is configured to covalently bond the anchoring strand to the probing strand upon application of light energy, and a tag bound to the probing strand, wherein the tag is configured to conjugate to a detectable label.

In general, in one embodiment, a photoreactive probe, including a nucleic acid anchoring strand, wherein the anchoring strand can include a bait attachment site, a nucleic acid probing strand, wherein the probing strand forms a double-stranded structure with the anchoring strand along a complementary sequence, a light-activated warhead disposed in the probe, wherein the light-activated warhead is configured to covalently bond the anchoring strand to the probing strand upon application of light energy, and a tag bound to the probing strand, wherein the tag is configured to conjugate to a detectable label.

This and other embodiments can include one or more of the following features. The anchoring strand and the probing strand include DNA, RNA, or DNA and RNA. The anchoring strand is longer than the probing strand. The anchoring strand and the probing strand have at least 6 complementary nucleotides in a continuous row and at least 20 complementary nucleotides in total. The double-stranded structure is at least 10 nucleotides in length. A melting temperature Tm of the double-stranded structure is from 52° C. to 60° C. A melting temperature Tₘ of the double-stranded structure is at least 50° C. The anchoring strand and the probing strand are at least 10 nucleotides in length, at least 20 nucleotides in length, or at least 30 nucleotides in length. The anchoring strand and the probing strand are no more than 40 nucleotides in length, no more than 50 nucleotides in length, or no more than 60 nucleotides in length. Each of the anchoring strand and the probing strand are between 15 nucleotides in length and 40 nucleotides in length. The anchoring strand can include a primary sequence and the tag is on the same side of the primary sequence as the bait attachment site, relative to the cleavable site. The probe is attached to a bait molecule at the bait attachment site. The probe is covalently attached to the bait molecule. The bait molecule can include an antibody, a CLIP-tag, a HaloTag, protein A, protein G, protein L, an RNA molecule, a small molecule, or a SNAP-tag. The bait molecule can include an antibody. The bait molecule can include a secondary antibody.

The tag can include a biotin derivative, a CLIP-tag, a click chemistry tag, digoxigenin, a HaloTag, a peptide tag, or a SNAP-tag. The biotin derivative includes the moiety of

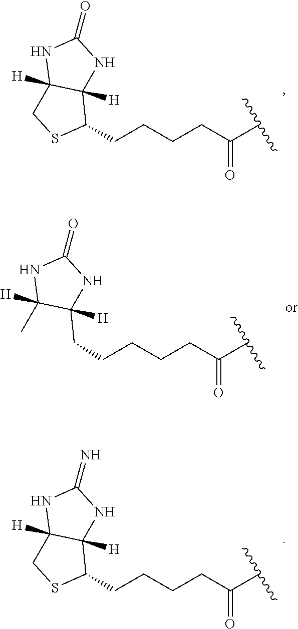

The light-activated warhead can include a nucleobase. The light-activated warhead can include a thymine-specific warhead. The light-activated warhead can include a nucleobase-specific psoralen, including the moiety of

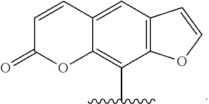 [SMILES: *c1c2occc2cc2ccc(=O)oc12]

The light-activated warhead can include a nucleobase-specific 3-cyanovinylcarbazole nucleoside (CNVK), including the moiety of

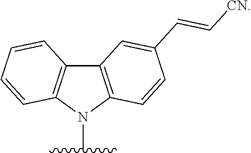 [SMILES: *n1c2ccccc2c2cc(/C=C/C#N)ccc21]

The light-activated warhead can include a nucleobase-specific trioxsalen, including the moiety of

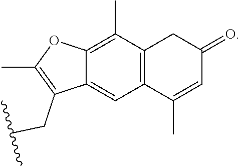 [SMILES: *Cc1c(C)oc2c(C)c3c(cc12)C(C)=CC(=O)C3]

In general, in one embodiment, a method for photoactivated labeling including conjugating any photoreactive probe above to a biomolecule in a biological sample, conjugating a bait molecule to a target biomolecule in the biological sample to crosslink the probe and target biomolecule, delivering optical radiation to a first region of the biological sample to activate the light-activated warhead and covalently bond the anchoring strand to the probing strand, removing the probing strand away from the rest of the probe and hybridizing a competing strand to the anchoring strand in a second region of the biological sample that has not been treating with optical radiation, and removing the cleaved and unbound probe from the biological sample.

In general, in one embodiment, an analytical method, including delivering a photoreactive probe to a biological sample, the probe including a nucleic acid anchoring strand and a nucleic acid probing strand, wherein the probing strand forms a double-stranded structure with the anchoring strand along a complementary sequence, selectively illuminating a first region of the biological sample to thereby activate a light-activated warhead disposed in the probe and covalently bond the probing strand to the anchoring strand in the first region, and not illuminating a second region of the biological sample such that the probing strand and anchoring strand are not covalently bonded in the second region, unwinding the probing strand away from the rest of the probe in the second region, delivering a competing nucleic acid strand to the first and second regions, wherein the competing nucleic acid strand is configured to compete with the probing strand for binding to the anchoring strand, and replacing the probing strand with the competing nucleic acid strand in probes in the second region, but not in the first region, wherein the covalent bonding between the probing strand and the anchoring strand in the first region prevents the competing strand from replacing the probing strand in the first region.

This and other embodiments can include one or more of the following features. The probe further can include a tag bound to the probing strand, wherein the tag is configured to conjugate to a detectable label. The step of conjugating a detectable label with the tag and detectably proximity labeling neighbors proximal the target biomolecule by detectable label activity. The step of detectably proximity labeling can include photoselectively proximity labeling a region less than 300 nm, less than 200 nm, or less than 100 nm in diameter or in a longest dimension. A melting temperature Tₘ of the double-stranded structure prior to cleaving the cleavable site is at least 50° C. A melting temperature Tm of the double-stranded structure prior to cleaving the cleavable site is from 52° C. to 60° C. The method can further include wherein (i) the probe can include a double-stranded structure after the cleaving step, (ii) the probe can include a double-stranded structure after the replacing step, and (iii) a melting temperature Tₘ of the double-stranded structure in the second region after cleaving the cleavable site is lower than a melting temperature Tm of the double-stranded structure in the second region after the replacing step in the second region. The melting temperature Tm of the double-stranded structure in the second region after cleaving the cleavable site is from 26° C. to 34° C. The melting temperature Tm of the double-stranded structure in the second region after the replacing step in the second region is from 44° C. to 53° C. The replacing step is performed at a temperature higher than the melting temperature Tₘ of the double-stranded structure in the second region after cleaving the cleavable site and lower than the melting temperature Tₘ of the double-stranded structure in the second region after the replacing step. A primary sequence of the anchoring strand, a primary sequence of the probing strand, and/or a primary sequence of the competing strand are bioorthogonal with naturally occurring nucleic acid chains in the biological sample, such that there is no sequence matches more than 10 nucleotides between endogenous and the probing strands or the anchoring strands.

The step of removing the probing strand away from the rest of the probe in the second region can include digesting the probing strand with an exonuclease. The step of removing the probing strand away from the rest of the probe in the second region can include treating the sample with an unwinding factor. The step of removing the probing strand away from the rest of the probe in the second region can include treating the sample with an unwinding factor including a helicase, a small molecule, or increased temperature. The step of selectively illuminating to activate a light-activated warhead can include activating a nucleobase warhead. The step of selectively illuminating to activate a light-activated warhead can include activating a thymine-specific warhead. The step of selectively illuminating the biological sample can include illuminating from an imaging lighting source of an image-guided microscope system, the method further including imaging the illuminated sample with a controllable camera, acquiring with the camera at least one image of subcellular morphology of the biological sample in a first field of view, processing the at least one image and determining a region of interest in the sample based on the processed image, and obtaining coordinate information of the region of interest. The step of removing unbound probe from the first and second regions. The step of selectively illuminating can include illuminating a region for 25 μs/pixel to 400 μs/pixel, for 50 us/pixel to 300 μs/pixel, or for 75 μs/pixel to 200 μs/pixel. The step of selectively illuminating can include illuminating with a power intensity of from 100 mW to 300 mW. The detectable label can include a catalytic label. The biological sample can include at least one, at least 100, at least 1000 or at least 10,000 live or fixed cells. The biological sample can include fixed cells, tissues, cell extracts, or tissue extracts. The step of selectively illuminating can include illuminating a zone defined by point spread function. The biological sample is disposed on a microscope stage, the method further including removing at least a portion of the illuminated region of the biological sample from the stage. The step of subjecting the sample to mass spectrometry analysis or sequencing analysis. The tag can include a biotin derivative, a CLIP-tag, a click chemistry tag, digoxigenin, a HaloTag, a peptide tag, or a SNAP-tag.

In general, in one embodiment, kit for labeling biomolecules including any of the photoreactive probes above in a first container, and an instructional material. This and other embodiments can include one or more of the following features. A competing strand, wherein the competing strand is complementary to the anchoring strand. The competing strand and anchoring strand form a double-stranded structure, the structure has a melting temperature Tm of from 44° C. to 53° C.

In general, in one embodiment, a kit for probe generation, including a nucleic acid anchoring strand, wherein the anchoring strand can include a bait attachment site, a nucleic acid probing strand, wherein the probing strand forms a double-stranded structure with the anchoring strand along a complementary sequence, a light-activated warhead, wherein the light-activated warhead is configured to covalently bond the anchoring strand to the probing strand upon application of light energy, and a tag bound to the probing strand, wherein the tag is configured to conjugate to a detectable label. One aspect of the disclosure provides a kit for photoreactive labeling, including one or more of a nucleic acid anchoring strand and a nucleic acid probing strand. In these and other embodiments, the anchoring strand can include a bait attachment site covalently attached to a bait molecule. In these and other embodiments, the probing strand is configured to form a double-stranded structure with the anchoring strand along a complementary sequence. These and other embodiments can include a light-activated warhead disposed in the probing strand, wherein the light-activated warhead is configured to covalently bond the anchoring strand to the probing strand upon application of light energy; and a tag bound to the probing strand, wherein the tag is a detectable label.

In these and other embodiments, the nucleic acid in the anchoring strand and the nucleic acid in the probing strand can be DNA, RNA, or both DNA and RNA.

In these and other embodiments, the anchoring strand is longer than the probing strand, and the anchoring strand and the probing strand have at least 6 complementary nucleotides in a continuous row and at least 20 complementary nucleotides in total. In these and other embodiments, the double-stranded structure is at least 10 nucleotides in length.

In these and other embodiments, a melting temperature Tm(1) of the double-stranded structure is at least 50° C. In these and other embodiments, the melting temperature Tm(1) of the double-stranded structure is from 52° C. to 60° C. In these and other embodiments, each of the anchoring strand and the probing strand are between 15 nucleotides and 40 nucleotides in length.

In these and other embodiments, the bait molecule can include a CLIP-tag, a HaloTag, SNAP-tag, protein A, protein G, protein L, or an RNA molecule.

In these and other embodiments, the bait molecule is a primary antibody or a secondary antibody.

In these and other embodiments, the tag can include a biotin derivative, digoxigenin, a click chemistry tag, a CLIP-tag, a HaloTag, or a SNAP-tag.

In these and other embodiments, the tag is a biotin derivative and the biotin derivative a moiety of

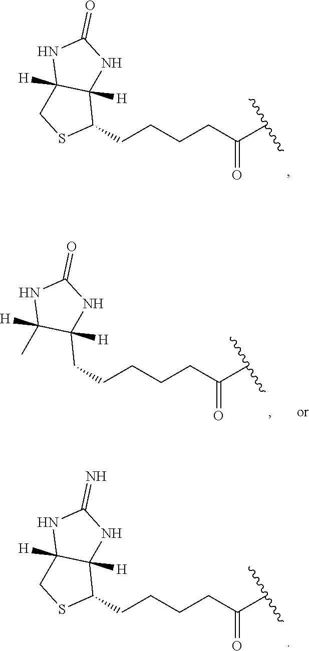

In these and other embodiments, the light-activated warhead can include a thymine-specific warhead. In these and other embodiments, the light-activated warhead is a nucleobase-specific psoralen, including a moiety of

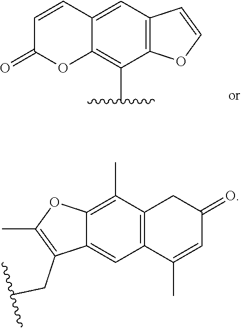 [SMILES: *c1c2occc2cc2ccc(=O)oc12 ; *Cc1c(C)oc2c(C)c3c(cc12)C(C)=CC(=O)C3]

In these and other embodiments, the light-activated warhead is a nucleobase-specific azide. In these and other embodiments, the light-activated warhead is a nucleobase-specific 3-cyanovinylcarbazole nucleoside (CNVK), including a moiety of

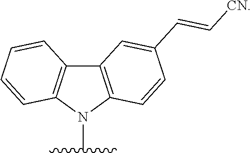 [SMILES: *n1c2ccccc2c2cc(/C=C/C#N)ccc21]

In these and other embodiments, the light-activated warhead can include a nucleobase-specific diazirine, including a moiety of

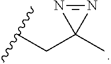 [SMILES: *CC1(C)N=N1]

In these and other embodiments, when the nucleic acid anchoring strand and the nucleic acid probing strand bind to each other along the complementary sequence to form the double-stranded structure, the double-stranded structure can include a cleavable site, wherein the anchoring strand and the probing strand are configured to break at the cleavable site.

In these and other embodiments, the cleavable site can include a restriction enzyme site or another endonuclease site.

In these and other embodiments, cleaving the double-stranded site at the cleavable site results in the formation of two smaller double-stranded structures, wherein a melting temperature Tm(2) of a first of the two smaller double-stranded structures is from 26° C. to 34° C., wherein the first of the smaller double-stranded structures does not contain the light-activated warhead or the tag.

Another aspect of the disclosure provides a photoselective labeling method that can include one or more (or all of) the steps of delivering a nucleic acid anchoring strand to a biological sample, wherein the anchoring strand can include a bait attachment site covalently attached to a bait molecule, and binding the bait molecule to a molecule of interest in the biological sample; removing unbound anchoring strand from the biological sample; delivering a nucleic acid probing strand to the biological sample, the probing strand can include a light-activated warhead disposed therein, and a tag bound to the 3′ end of the probing strand, wherein the probing strand forms a double-stranded structure with the anchoring strand along a complementary sequence to form a photoreactive probe; selectively illuminating a first region of the biological sample to thereby activate the light-activated warhead disposed in the probing strand and covalently bond the probing strand to the anchoring strand of the probe in the first region to achieve a photoselective labeling, and not activating the light-activated warhead in a second region of the biological sample such that the probing strand and anchoring strand are not covalently bonded in the probe in the second region; heating the biological sample above a melting temperature Tm(1) such that the probing strand separates from the anchoring strand in probe in the second region of the biological sample to form unbound probing strand, while the probing strand and anchoring strand remain in the double-stranded structure in the first region of the biological sample; and removing the unbound probing strand from the biological sample.

In these and other photoselective labeling methods, the melting temperature Tm(1) of the double-stranded structure of the photoreactive probe is from 52° C. to 60° C.

Another aspect of the disclosure provides a photoselective labeling method including one or more of the steps of: delivering a nucleic acid anchoring strand to a biological sample, wherein the anchoring strand can include a bait attachment site covalently attached to a bait molecule, and the bait molecule binds a molecule of interest in the biological sample; removing unbound anchoring strand from the biological sample; delivering a nucleic acid probing strand to the biological sample, the probing strand can include a light-activated warhead disposed in the probing strand, and a tag bound to the 3′ end of the probing strand, wherein the probing strand forms a double-stranded structure with the anchoring strand along a complementary sequence to become a photoreactive probe; selectively illuminating a first region of the biological sample to thereby activate the light-activated warhead disposed in the probing strand and covalently bond the probing strand to the anchoring strand of the probe in a first region to achieve a photoselective labeling, and not activating the light-activated warhead in a second region of the biological sample such that the probing strand and anchoring strand are not covalently bonded in the probe in the second region; delivering an exonuclease to digest the probing strand from 5′ to 3′ direction, wherein after the digesting, a remaining double-stranded structure has a melting temperature Tm(2); heating the biological sample above a melting temperature Tm(2) such that the probing strand separates from the anchoring strand in probe in the second region of the biological sample to form unbound probing strand, while the probing strand remains in the double-stranded structure with the anchoring strand in probe in the first region of the biological sample; and removing the unbound probing strand from the biological sample.

These and other embodiments may include wherein the melting temperature Tm(2) of the double-stranded structure of the photoreactive probe is from 26° C. to 34° C.

Another aspect of the disclosure provides a photoselective labeling method including one or more (or all of) the steps of: delivering a nucleic acid anchoring strand to a biological sample, wherein the anchoring strand can include (1) a bait attachment site covalently attached to a bait molecule, and the bait molecule binds a molecule of interest in the biological sample; and (2) a first cleavable sequence; removing unbound anchoring strand from the biological sample; delivering a nucleic acid probing strand to the biological sample, the probing strand can include a light-activated warhead disposed in the probing strand, a second cleavable sequence, and a tag bound to the 3′ end of the probing strand, wherein the probing strand forms a double-stranded structure with the anchoring strand along a complementary sequence to become a photoreactive probe with a cleavable site; selectively illuminating a first region of the biological sample to activate light-activated warhead disposed in the photoreactive probe and thereby covalently bond the probing strand to the anchoring strand in a first region to achieve a photoselective labeling, and not illuminating a second region of the biological sample such that the probing strand and anchoring strand are not covalently bonded together in the second region; cleaving the cleavable site in the double-stranded region of the photoreactive probe by application of a cleaver, wherein after the cleaving, a remaining double-stranded structure has a melting temperature Tm(2); heating the biological sample above a melting temperature Tm(2) such that the probing strand separates from the anchoring strand in probe in the second region of the biological sample to form unbound probing strand, while the probing strand remains in the double-stranded structure with the anchoring strand in probe in the first region of the biological sample; removing the cleaved and unbound probe from the biological sample.

In these and other embodiments, the melting temperature Tm(2) of the double-stranded structure of the photoreactive probe is from 26° C. to 34° C.

In these and other embodiments, cleaving the cleavable site can include cutting the cleavable site with a restriction enzyme or another endonuclease.

### BRIEF DESCRIPTION OF THE DRAWINGS

A better understanding of the features and advantages of the methods and apparatuses

described herein will be obtained by reference to the following detailed description that sets forth illustrative embodiments, and the accompanying drawings of which:

FIG. 1 shows a schematic depiction of a system useful for photoselective spatial tagging and proximity labeling of cells on a substrate.

FIG. 2A shows a schematic illustration of a multifunctional photoreactive and cleavable probe. The photoreactive and cleavable probe has a multivalent core with a plurality of attachment sites. A tag, a cleavable linker, and a light activated warhead are bound to the attachment sites on the probe. FIG. 2B schematically illustrates a proximity labeling system that can be used to label biomolecules in a small region of interest using the probe shown in FIG. 2A.

FIG. 2C shows a schematic illustration comparing the results of direct photochemical labeling with photo-assisted enzymatic proximity labeling using the multifunctional photoreactive and cleavable probes described herein to label biomolecules in small region of interest (ROI). The probes are shown in FIG. 2B.

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A).

With mild cleavage conditions the protein structure is retained, and the reaction is bioorthogonal, while with harsh cleavage conditions, the protein denatures, and the reaction is non-bioorthogonal.

FIGS. 4A-4K show examples of tags that can be used in the photoreactive and cleavable probes described herein. The tags are configured to interact with a label for labeling biomolecules neighboring a target molecule of interest. FIG. 4A-FIG. 4E show examples of click chemistry tags that can be used with the probes. FIGS. 4F-4H show examples of biotin derivatives that can be used with the probes. FIG. 4I shows a digoxigenin moiety. FIG. 4J shows a peptide tag (SEQ ID NO: 1). FIG. 4K shows a SNAP-tag.

FIGS. 5A-5E show examples of site-specific cleavable linkers that can be used in the photoreactive and cleavable probes described herein. FIG. 5A shows an azobenzene moiety. FIG. 5B shows a boronic ester moiety. FIG. 5C shows a Dde moiety. FIG. 5D shows a DNA oligomer. FIG. 5E shows a peptide moiety.

FIGS. 6A-6E shows examples of bait molecules that can be used in the photoreactive and cleavable probes described herein to conjugate (e.g., covalently or non-covalently) with a molecule of interest in a sample. FIG. 6A shows an antibody that can be used a bait molecule. FIG. 6B shows a nucleic acid portion that can be used as a bait molecule. FIG. 6C shows a representation of a functional protein that can be used as a bait molecule. FIG. 6D shows small molecules/drugs can be used as bait molecules. By way of example, erlotinib is shown. FIG. 6E shows a CLIP-tag and other members of self-labeling moieties could be used (e.g., HaloTag or SNAP-Tag).

FIGS. 7A-7I show examples of photoactive warheads that can be used in the photoreactive and cleavable probes described herein.

FIG. 8A-8G show additional examples of linkers that can be used in the photoreactive and cleavable probe described herein.

FIGS. 9A-9G show examples of photoreactive and cleavable probes. The probes have multivalent cores with a plurality of attachment sites. A tag is bound to one of attachment sites, a cleavable linker is bound to another attachment site, and a light activated warhead is bound to another of the attachment sites on the probe.

FIGS. 10A-10B schematically illustrate peptide-based photoreactive and cleavable probes. These probes have a peptide region cleavable by a peptide cleavage reagent, such as by a protease that recognizes a specific peptide sequence. FIG. 10A shows an example of a peptide-based probe with a tag and warhead on the N-terminal end of the peptide region. FIG. 10B shows an example of a peptide-based probe with a tag and warhead on the C-terminal end of the peptide region. FIGS. 10A-10B also show probes with an additional, flexible linker and an optional clickable amino acid. Additional linkers (also referred to as spacers) can play a role in bridging the attachment sites between bait molecules and photoreactive and cleavable probe. The distance between the probe and bait can be controlled by applying linkers with different spatial lengths.

FIGS. 10C-10I show examples of reactive or clickable amino acids that can be used with the probes shown in FIGS. 10A and 10B. A clickable amino acid may be useful for attaching a bait molecule, such as an antibody.

FIGS. 10J-10Q show examples of peptide-based photoreactive and cleavable probes schematically illustrated in FIGS. 10A-10B. The cleavage sites for the human rhinovirus 3C (HRV 3C) protease, tobacco etch virus (TEV) protease, and thrombin are shown with arrows. Figure discloses SEQ ID NOS 18-25, respectively, in order of appearance.

FIGS. 11A-11D illustrate methods and steps used to synthesize the photoreactive and cleavable probes described herein. The methods create probes with a tag, a cleavable linker, and a light activated warhead. Figure discloses SEQ ID NOS 20 and 26, respectively, in order of appearance.

FIG. 12A schematically illustrates a photoreactive and cleavable probe conjugated to an antibody bait.

FIG. 12B and FIG. 12C schematically illustrates a reaction scheme for performing photoselective tagging of a molecule using a photoreactive and cleavable probe conjugated to an antibody bait for tagging proteins in the cell nucleolus. FIG. 12B illustrates how the reaction proceeds using controlled light. FIG. 12B illustrates how the cleavable probes are cleaved to reduce background in non-lighted areas.

FIG. 12D shows results from using the reaction schemes shown in FIG. 12A and FIG. 12B. The nucleolin protein is specifically tagged in the presence of light (top and right panels) but is not tagged in the absence of light (bottom panel).

FIG. 13A represents a schematic diagram of an imaging-guided system.

FIG. 13B depicts the optical path of the image-guided system of FIG. 13A.

FIG. 14A represents a schematic diagram of another imaging-guided system.

FIG. 14B depicts the optical path of the image-guided system of FIG. 14A.

FIG. 15A represents a schematic diagram of yet another imaging-guided system. FIG. 15B depicts the optical path of the image-guided system of FIG. 15A.

FIG. 16A schematically illustrates a photoreactive and cleavable probe with a nucleic acid anchoring strand, a nucleic acid probing strand, a light-activated warhead, and a tag.

FIG. 16B schematically illustrates the probe in FIG. 16A conjugated to an antibody.

FIG. 16C schematically illustrates use of the probe-antibody conjugate illustrated in FIG. 16B, such as for labelling target biomolecules in a sample.

FIG. 17A illustrates an example of the probe and method shown in FIG. 16C. FIGS. 17B-17D schematically illustrate the probe structures shown in FIG. 17A along with melting temperatures for different probe structures. Figure discloses SEQ ID NOS 27-31 and 30, respectively, in order of appearance.

FIG. 18A illustrates steps for removing a tag from a molecule in a non-light activated region of a sample. The tag is removed using an enzyme treatment followed by strand displacement and replacement. Figure discloses SEQ ID NOS 27-28, 28, 32, and 28, respectively, in order of appearance.

FIGS. 18B-18D show another photoreactive probe. FIG. 18B schematically illustrates a probe and FIG. 18C and FIG. 18D illustrate altered versions of the probe shown in FIG. 18B. The different melting temperatures for the different versions can be used for removing a tag from molecules in part of a sample.

FIG. 19A and FIG. 19B schematically illustrate another photoreactive probe that can be used for removing a tag from molecules in part of a sample. An unwinding factor is used to remove the tag.

FIG. 19C shows steps in tagging molecules in a light-activated region of a sample and removing a tag from molecules in a non-light-activated region.

FIG. 20A illustrates steps in a method for removing a tag from a molecule in a non-light-activated region of a sample. The method uses an unwinding factor, strand displacement, and strand replacement to remove the tag. FIG. 20B C and FIG. 20C illustrate different melting temperatures for the altered versions of the probe shown in FIG. 20A. The different melting temperatures facilitate strand displacement and replacement. Photoactivated probe remains bound in the light activated region. Figure discloses SEQ ID NOS 27-28, 28, 32, and 28, respectively, in order of appearance.

FIG. 20D illustrates steps in a method for removing a tag from a molecule in a non-light-activated region of a sample. The method employs a heating step to melt unbound probe and remove the tag from molecules in a non-light activated region. Photoactivated probe remains bound in the light activated region.

FIG. 21A-21C shows labeling of biological samples (stress granules) using a photoreactive probe and methods disclosed herein. FIG. 21A shows the fluorescent image of control sample, which was incubated with probing strand without warhead. FIG. 21A shows low labeling background. FIG. 21B shows the fluorescent image of a biological sample which was incubated with probing strand with warhead. Small spots can be clearly observed within the cell boundary. FIG. 21C shows the fluorescent image of positive sample after amplification of the biotin signal of probing strand. The intensity of fluorescence and numbers of stress granule spot are largely amplified by 5˜10 times.

### DETAILED DESCRIPTION

Described herein are systems, compositions, and methods useful for identifying, tagging, obtaining, and analyzing biomolecules and their neighboring biomolecules. The compositions and methods may be particularly useful for analyzing biomolecule interactions in biological samples, such as analyzing proteins, nucleic acids, carbohydrates, or lipids in cell or tissue samples. The compositions and methods utilize photoreactive and cleavable probes (e.g., bioorthogonally or mildly cleavable or enzyme-specific cleavage) that can label biomolecules and their neighboring biomolecules, while largely maintaining naturally occurring molecular structure in the biomolecules. The photoreactive and mildly cleavable probes described herein may be particularly useful for specifically labeling subsets of biomolecules in subcellular regions of cells using an image guided microscope with precision illumination control such as the system described in U.S. Patent Publication No. 2018/0367717, to enable automatic labeling of cellular biomolecules of interest. The probes can be used for in situ tagging of biomolecules such as proteins inside cells or tissues and that can be followed by tag transfer or proximity labeling such as using Tyramide Signal Amplification (TSA). The biomolecules can be further analyzed by analytical techniques such as mass spectrometry and sequencing. These probes may be especially useful for performing omics studies, such as genomics, proteomics, and transcriptomics, and for finding relevant biomarkers for diagnosis and treatment.

### Abbreviations and Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Amino acids described herein may be conservatively substituted so long as conservatively substituted peptide enables the desired function (such as recognition by a protease). Examples of conservative substitutions include Thr, Gly, or Asn for Ser and His, Lys, Glu, Gln for Arg. Conservative substitutions are described in e.g., Molecular Cloning: A Laboratory Manual, Fourth Edition, Green and Sambrook, eds., Cold Spring Harbor Laboratory Press, Cold Spring Harbor 2014, as well as corrections and updates thereto.)

The term “antibody” refers to immunoglobulin and related molecules and includes monoclonal antibodies, polyclonal antibodies, monomers, dimers, multimers, multispecific antibodies (e.g., bispecific antibodies), heavy chain only antibodies, three chain antibodies, single chain Fv, nanobodies, etc., and also includes antibody fragments. An antibody may be a polyclonal or monoclonal or recombinant antibody. Antibodies may be murine, human, humanized, chimeric, or derived from other species. As used herein, when an antibody or other entity “specifically recognizes” or “specifically binds” an antigen or epitope, it preferentially recognizes the antigen in a complex mixture of proteins and/or macromolecules and binds the antigen or epitope with affinity, which is substantially higher than to other entities not displaying the antigen or epitope.

The term “aryl” refers to an aromatic ring system having a single ring (e.g., a phenyl group or a substituted phenyl group). Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

The term “bait molecule” refers to a molecule that specifically interacts with a molecule of interest, which may be referred to as a target (or prey). Examples of bait molecules include an antibody, CLIP-tag, a drug, a nucleic acid, a fluorescent in situ hybridization (FISH) probe, protein A, protein G, protein L, protein A/G, protein A/G/L, another small molecule, and a SNAP-tag.

The term “binding” refers to a first moiety physically interacting with a second moiety, wherein the first and second moieties are in physical contact with one another.

The term “bioorthogonal” refers to not interfering with or not interacting with biology (e.g., being inert to biomolecules).

The term “bioorthogonal reaction” or “bioorthogonal cleavage reaction” refers to a reaction that proceeds under physiologically relevant conditions and compatibility with naturally occurring functional groups and typically with fast kinetics, tolerance to an aqueous environment, and high selectively. A bioorthogonal reaction proceeds under conditions configured to maintain naturally occurring molecular structure, such as protein folding or three-dimensional structure. A bioorthogonal reaction does not break cross-links between different regions of polypeptide chains in endogenous or sample proteins or peptides. For example, a bioorthogonal reaction does not break covalent bonds in naturally occurring functional groups (e.g., disulfide (—S—S-bonds) in cysteine side chains). A bioorthogonal cleavage linker or a cleavage linker in a bioorthogonal cleavage probe is configured for bioorthogonal cleavage, such as being compatible with using an enzyme or bond-specific chemicals configured to proceed bioorthogonally without breaking covalent bonds in naturally occurring functional groups.

The term “biotin derivative” refers to a biotin moiety, including biotin and variations of biotin, such as biotin with an open ring or substitutions. Typically, a biotin derivative is easily detectable with a biotin-binding entity or protein, such as avidin, NeutrAvidin, or streptavidin.

The term “catalyzed reporter deposition” (CARD) refers an enzyme catalyzed deposition of a detectable molecule on or near target biomolecules (e.g., carbohydrates, lipids, nucleic acids, or proteins). In some embodiments, the enzyme in an enzyme catalyzed deposition is horseradish peroxidase (HRP) and the detectable molecule is tyramide or digoxygenin (DIG).

The term “cleavable linker bond” refers to the chemical bond in a cleavable linker configured to be specifically cleaved by a cleavage reagent. Typically, a cleavable linker bond refers to a single bond; however, in some variations, a cleavable linker bond can refer to more than one bond, such as in the case of a double-stranded DNA cleavable linker cleavable by an endonuclease in which two strands of DNA are cleaved.

The term “click chemistry” refers to a chemical approach that easily joins molecular building blocks. Typically, click chemistry reactions are efficient, high-yielding, reliable, create few or no byproducts, and are compatible with an aqueous environment or without an added solvent. An example of click chemistry is cycloaddition, such as the copper(I)-catalyzed [3+2]-Huisgen 1,3-dipolar cycloaddition of an alkyne and azide leading to the formation of 1,2,3-triazole or Diels-Adler reaction. Click chemistry also includes copper free reactions, such as a variant using substituted cyclooctyne (see e.g., J. M. Baskin et al., Proc. Natl. Acad. Sci. U.S.A. 2007 Oct. 23, 104 (43), 16793-16797.) Other examples of click chemistry are nucleophilic substitutions; additions to C-C multiple bonds (e.g., Michael addition, cpoxidation, dihydroxylation, aziridination); and nonaldol like chemistry (e.g., N-hydroxysuccinimide active ester couplings). Click chemistry reactions can be bioorthogonal reactions, but do not need to be.

The term “conjugate” refers to a process by which two or more molecules specifically interact (e.g., covalently or non-covalently). In some embodiments, a tag and a label conjugate. In some embodiments, a bait and a cleavable probe conjugate.

The term “conjugatable” refers to a molecule that can specifically come together with another molecule to which it can be conjugated. In some embodiments, a bait is conjugatable to a biomolecule of interest. In some embodiments a cleavable probe is conjugatable to a label.

The term “detectable label” refers to a compound or composition which is or is configured to be conjugated directly or indirectly to a molecule. The label itself may be detectable and be a directly detectable label (such as, e.g., fluorescent labels such as fluorescent chemical adducts, radioisotope labels, etc.), or the label can be indirectly detectable (such as, e.g., in the case of an enzymatic detectable label, the enzyme may catalyze a chemical alteration of a substrate compound or composition and the product of the reaction is detectable). Examples of detectable labels include e.g., a biotin label, a fluorescent label, horseradish peroxidase, an immunologically detectable label (e.g., a hemagglutinin (HA) tag, a poly-histidine tag), another light emitting label, and a radioactive label. An example of an indirect label is biotin, which can be detected using a streptavidin detection method.

The term “enzymatic cleavage reaction” refers to cleavage or hydrolysis of bonds in molecules mediated by an enzyme. Typically, enzyme mediated reactions cleave covalent bonds and lead to the formation of smaller molecules.

The term “immunoglobulin-binding protein” refers to immunoglobulin-binding bacterial proteins and variations of immunoglobulin-binding bacterial proteins. Examples include protein A, protein G, protein L, protein A/G, and protein A/G/L. Protein A and protein G and are bacterial proteins originally obtained from Staphylococcus aureus and Group G Streptococci, respectively, and have high affinity for the Fc region of IgG type antibodies. Protein A/G combines the binding domains of protein A and protein G. Protein A/G/L combines binding domains of protein A, protein G, and protein L. Immunoglobulin-binding proteins bind to specific domain of antibodies.

The term “instructional material” includes a publication, a recording, a diagram, a link, or any other medium of expression which can be used to communicate the usefulness of one or more compositions of the invention for its designated use. The instructional material of a kit of the invention may, for example, be affixed to a container which contains the composition or components or be shipped together with a container which contains the composition or components. Alternatively, the instructional material may be shipped separately from a container with the intention that the instructional material and a composition or component be used cooperatively by the recipient.

The term “label” refers to a molecule which produces or can be induced to produce a detectable signal. In some embodiments, a label produces a signal for detecting a neighboring biomolecule. Examples of labels that can be used include avidin labels, NeutrAvidin labels, streptavidin labels to detect a biotin tag.

The term “linker” refers to a structure which connects two or more substructures. A linker has at least one uninterrupted chain of atoms extending between the substructures. The atoms of a linker are connected by chemical bonds, typically covalent bonds.

The term “light activated warhead” refers to a group with a light activated moiety. Examples of light activated warheads include aryl azides, benzophenone, and diazirines. Once activated, a light activated warhead can bind to a binding partner.

The term “mass spectrometer” refers to an instrument for measuring the mass-to-charge ratio of one or more molecules in a sample. A mass spectrometer typically includes an ion source and a mass analyzer. Examples of mass spectrometers includes matrix assisted laser desorption ionization (MALDI), continuous or pulsed electrospray (ES) ionization, ionspray, magnetic sector, thermospray, time-of-flight, and massive cluster impact mass spectrometry.

The term “mass spectrometry” refers to the use of a mass spectrometer to detect gas phase ions.

The term “mass spectrometry analysis” includes linear time-of-flight (TOF), reflectron time-of-flight, single quadruple, multiple quadruple, single magnetic sector, multiple magnetic sector, Fourier transform, ion cyclotron resonance (ICR) or ion trap.

The term “melting temperature” or Tm of a nucleic acid refers to the temperature at which 50% of double stranded nucleic acid is changed to single stranded nucleic acid.

The term “photoactivated” or “light activated” refers to excitation of atoms by means of radiant energy (e.g., by a specific wavelength or wavelength range of light, UV light, etc.). In some examples, a photoactivated molecule forms a covalent linkage with another molecule or another part of itself within its immediate vicinity.

The term “peptide” refers to a polymer in which the monomers are amino acids and the monomers are joined together through amide bonds. A peptide is typically at least 2, least 5, least 10, least 20, least 50, least 100, or at least 500 or more amino acids long.

The term “primary sequence” of a molecule refers to a linear sequence of units that make up the primary structure of the molecule. The “primary sequence” of a nucleic acid refers to the linear sequence of nucleotides that make up the primary structure of the nucleic acid.

The term “photoreactive group” refers to a functional moiety, which, upon exposure to light (e.g., a specific wavelength or wavelength range of light, UV light, etc.) becomes activated. A photoreactive group typically forms a covalent linkage with a molecule within its immediate vicinity.

The term “proximity molecule” or neighboring molecule refers to a molecule that is near another molecule. A proximity molecule or neighbor molecule may conjugate to or interact with the molecule (e.g., covalently or non-covalently) or may be close by and not covalently bound to the molecule.

The term “prey” refers to a binding partner of a bait molecule. For example, if an antibody is a bait, a corresponding protein to which the bait molecule can bind is the corresponding prey. In some embodiments, a bait can bind with a single prey. In some embodiments, a bait can bind with more than one prey.

The term “protein tag” refers to peptide sequences of amino acids. Protein tags can typically be conjugated to a label. An example of a protein tag is a “self-labeling” tag. Examples of self-labeling tags include BL-Tag, CLIP-tag, covalent TMP tag, HALO-tag, and SNAP-tag. SNAP-tag is a ˜20 kDa variant of the DNA repair protein O6-alkylguanine-DNA alkyltransferase that specifically recognizes and rapidly reacts with benzylguanine (BG) derivatives. During a labeling reaction, the benzyl moiety is covalently attached to the SNAP-tag, releasing guanine. CLIP-tag is a variation of SNAP-tag configured to react specifically with O2-benzylcytosine (BC) derivatives rather than benzylguanine (BG).

The term “small molecule” refers to low molecular weight molecules that include carbohydrates, drugs, enzyme inhibitors, lipids, metabolites, monosaccharides, natural products, nucleic acids, peptides, peptidomimetics, second messengers, small organic molecules, and xenobiotics. Typically, small molecules are less than about 1000 molecular weight or less than about 500 molecular weight.

The term “tag” refers to a functional group, compound, molecule, substituent, or the like, that can enable detection of a target molecule. A tag can enable a detectable biological or physiochemical signal that allows detection via any means, e.g., absorbance, chemiluminescence, colorimetry, fluorescence, luminescence, magnetic resonance, phosphorescence, radioactivity. The detectable signal provided due to the tag can be directly detectable due to a biochemical or physiochemical property of the tag moiety (e.g., a fluorophore tag) or indirectly due to the tag interaction with another compound or agent. Typically, a tag is a small functional group or small organic compound. In some embodiments, the employed tag has a molecular weight of less than about 1,000 Da, 750 Da, 500 Da or even smaller.

The term “tagging” refers to the process of adding a tag to a functional group, compound, molecule, substituent, or the like. Typically, tagging enables detection of a target molecule.

The term “tyramide signal amplification” (TSA), refers to a catalyzed reporter deposition (CARD) an enzyme-mediated detection method that utilizes catalytic activity of an enzyme (e.g., horseradish peroxidase) to catalyze inactive tyramide to highly active tyramide. The amplification can take place in the presence of low concentrations of hydrogen peroxide (H₂O₂). In some examples, tyramide can be labeled with a detectable label, such as fluorophore (such as biotin or 2,4-dinitrophenol (DNP)).

The practice of the techniques described herein may employ, unless otherwise indicated, conventional techniques and descriptions of chemistry, biochemistry, cell biology, immunology, molecular biology (including cell culture, recombinant techniques, sequencing techniques) and organic chemistry technology which are explained in the literature in the field (e.g., Molecular Cloning: A Laboratory Manual, Fourth Edition, Green and Sambrook, eds., Cold Spring Harbor Laboratory Press, Cold Spring Harbor 2014, as well as corrections and updates thereto; John D. Roberts and Marjorie C. Caserio (1977) Basic Principles of Organic Chemistry, second edition. W. A. Benjamin, Inc., Menlo Park, CA.).

### Compositions

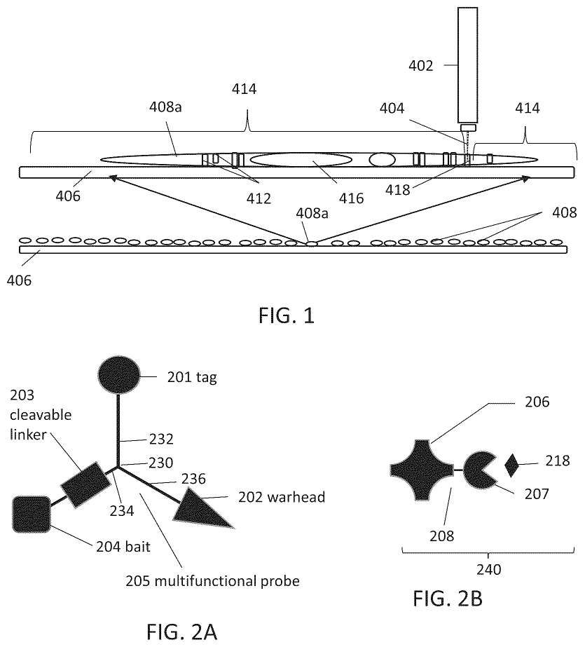

Described herein are compositions of matter including photoreactive and cleavable probes (e.g., bioorthogonally or mildly cleavable probes). The photoreactive and cleavable probes can advantageously be used with a microscope system, such as the systems described herein and in U.S. Patent Publication No. 2018/0367717 A1, to enable automatic labeling of cellular biomolecules proximal to a biomolecule of interest. The labeled molecules may be adjacent the biomolecule of interest or may be close-by but not adjacent, such as when intervening molecules are between the biomolecule of interest and cellular biomolecules for capture or analysis. Molecules that are close-by but not adjacent to a molecule of interest may be part of cell structure or otherwise contribute to a cell microenvironment of interest. FIG. 1 shows a schematic depiction of a system useful for photoselective spatial tagging and labeling. The bottom part of FIG. 1 shows substrate 406, such as a microscope stage, and a monolayer of plurality of cells 408 disposed on the substrate. In some embodiments, the surface of an entire substrate, or a portion of the substrate, can be analyzed using an automated microscope system to identify a region of interest. For example, a sample can be stained or labeled to identify a region of interest. The top part of FIG. 1 shows an expanded view of cell 408a, one of the plurality of cells 408. The cell 408a has a nucleus 416 and a plurality of different types of organelles 412, such as cell membranes, mitochondria, ribosomes, and vacuoles. Microscope system 402 selectively shines narrow band of light 404 onto region of interest (ROI) 418 for analysis of the region of interest 418. The illumination can be selective, and large regions 414 of the cell and substrate are not illuminated. As explained in more detail below, narrow band of light 404 activates a photorcactive and mildly cleavable probe in only the region of interest 418.

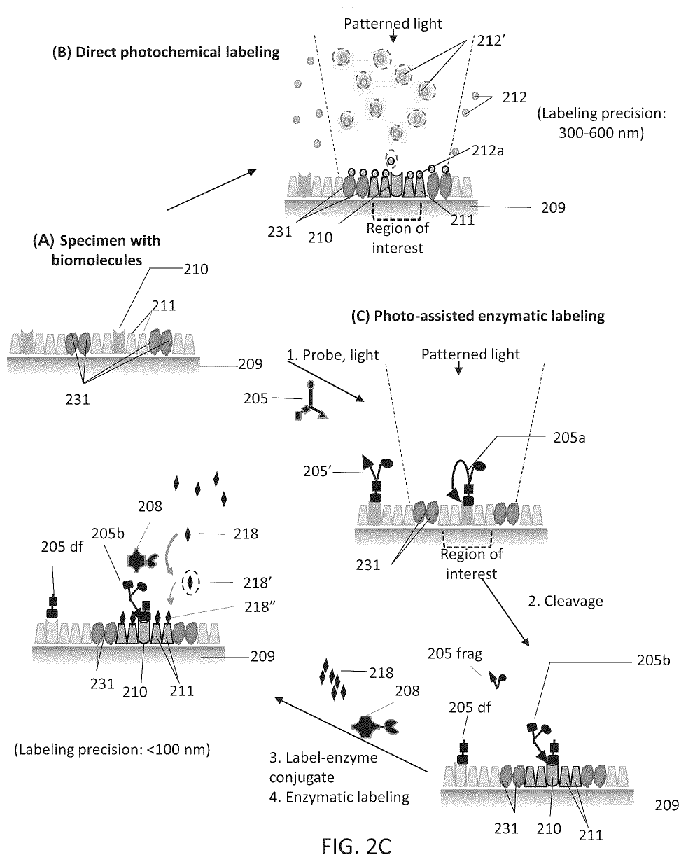

FIG. 2A schematically illustrates multifunctional probe 205 (also referred to interchangeably herein as multifunctional photoreactive and cleavable probe, photoreactive and cleavable probe, or probe unless specific context indicates otherwise). FIG. 2A shows multifunctional probe 205 has a multivalent core 230 with a plurality of attachment sites, first attachment site 232, second attachment site 234, and third attachment site 236. The multifunctional probe of FIG. 2A has tag 201 (circle) attached to first attachment site 232, cleavable linker 203 (rectangle) attached to second attachment site 234, and a light activated warhead (triangle) attached to third attachment site 236, thus forming a trivalent and trifunctional probe. Bait molecule 204 (rounded square) is attached to cleavable linker 203. FIG. 2B shows an example of a labeling system 240 that can be used with the multifunctional probe 205 shown in FIG. 2B to label biomolecules neighboring a target biomolecule of interest. Labeling system 240 includes labeling complex 208 with label 206 and enzyme or catalyst 207, and enzyme/catalyst substrate 218. In some embodiments, label 206 is NeutrAvidin and enzyme or catalyst 207 is peroxidase and utilizes peroxide (not shown) for activity. In this example, tag 201 and label 206 recognize one another and conjugate. Enzyme or catalyst 207 activates enzyme/catalyst substrate 218 and, once activated, activated enzyme/catalyst substrate 218 can bind to and detectably label biomolecules in its vicinity.

FIG. 2C shows a schematic illustration comparing the results of direct photochemical labeling with photo-assisted enzymatic labeling using the multifunctional photoreactive and cleavable probes described herein to label biomolecules in small region of interest (ROI). FIG. 2B shows a comparison of direct photochemical labeling (top, labeled Process B) and photo-assisted enzymatic labeling (bottom, labeled Process C) using the probes and systems described herein on a specimen with biomolecules (A). Prior to performing either Process B or Process C, a sample (e.g., a cell or tissue sample) containing a biomolecule of interest 210 (protein will be used herein by way of example, but other biomolecules could instead be analyzed) is analyzed and a region of interest identified. The sample can be pretreated, such as fixed and stained. For example, a sample can be fixed and stained with a cell stain (e.g., hematoxylin and eosin (H & E); Masson's trichrome stain), identified with an immunofluorescent labeled antibody recognizing a protein of interest or by other methods. Once the region of interest is identified, a complex of neighboring biomolecules within the region of interest is analyzed. As illustrated in Process B, the sample is treated with a direct photoreactive probe 212 and patterned light is directed to the sample and activate direct photoreactive probe 212 to form activated direct photoreactive probe 212 ′. The activated direct photoreactive probe 212′ is able to form complexes with other molecules with a close vicinity (show by the dotted circle in Process B. The activated direct photoreactive probe 212′ can diffuse and labels neighbor molecules 211 near the molecule of interest 210. However, the labeling diameter (300-600 nm) of direct photoactivation of photoreactive probes is spatially restricted by the diffraction limit of the light sources used. Additionally, since the photoreactive probe is free to diffuse, any proteins in the pathway of the patterned light can be labeled. Process B also shows it labels more distant biomolecules 231. The region labeled by activated direct photoreactive probe 212′, or labeled precision, covers a region of about 300-600 nm. This region can include biomolecules that are not in close proximity to protein of interest, and in some cases might lead to confusing, misleading or unhelpful results.

In contrast, in Process C, shown on the bottom of FIG. 2C, multifunctional probe 205 preconjugated with bait molecule (see FIG. 2A) recognizing the biomolecule of interest is delivered to the sample on substrate 209. As illustrated in Step 1, patterned light is also directed to the sample. However, here, patterned light activates the photoreactive warhead 202 which binds to molecules or moieties close by. In addition to the light directing a limited region of activation, the photoreactive warhead is constrained by its attachment to the probe 205 and the photoreactive warhead becomes attached to the biomolecule of interest. The attached probe 205a is now double-crosslinked to the biomolecule of interest (or close to it). FIG. 2C also shows Step 2 Cleavage, and the cleavable linker 203 is cleaved, such as by the addition of a protease if the cleavable linked is a cleavable peptide linker. Step 1 and Step 2 also show how background or unwanted labeling is reduced using the probes and methods described herein. In Step 1, a probe 205′ is attached to a biomolecule; however, since the probe 205′ is outside the light delivery region, photoreactive warhead 202 is not activated and does not bind to the biomolecule of interest. In Step 2, the probe 205′ is cleaved into two pieces, fragment 205 frag which is unbound and washed away in a washing step and 205 df which is defanged due to removal of tag 201 (which is washed away as part of unbound fragment 205 frag). Neither of the probe fragments 205df or 205 frag are able to label any biomolecules. The probe 205b is cleaved, but remains attached to the biomolecule of interest by double-crosslinking. In some variations, the probe 205c may be crosslinked to a bait molecule or another proximal biomolecule; however, the principle remains the same. The probe 205b contains tag 201, and as explained in more detail below, labels neighbor molecules.

Labeling system 240 includes labeling complex 208 with label 206 and enzyme or catalyst 207, and enzyme/catalyst substrate 218.

Excess probe is washed away with wash solution and single-crosslinked probes (c.g., in non-lighted areas) are removed through site-specific cleavage as described above. Steps 3 and 4 show labeling of the molecules near the molecule of interest 210 using labeling system 240 shown in FIG. 2B. Other labeling systems can also be used. By way of example, complex 208 conjugates with tag 201, the enzyme or catalyst 207 activates enzyme/catalyst substrate 218 to activated enzyme/catalyst substrate 218′. Since probe 205b is attached to molecule of interest 210, neighbor molecules 211 are labeled, while more distant molecule 231 is not. The cleavable linkers described herein can enable label transfer from the probe to neighbor molecules within a radius of <10 nm (referring to the size of the radius of the trifunctional (multifunctional) probe).

By photoselectively localizing enzyme or catalyst 207, such as peroxidase, near the molecule of interest and labeling the neighbor molecules 211 in the region of interest using the tagging and labeling just described, the coupling reaction can be localized to a region as small as <100 nm. In some variations, a larger region (e.g., up to about 200 nm, up to about 300 nm, up to about 400 nm) could be labeled. Furthermore, some molecules of interest in a sample have more one region of localization and hence interact with different molecular complexes in different locations simultaneously. The light-assisted tag transfer (e.g., tagging neighbor molecules) can be used successively in more than one location. For example, after applying light as shown in FIG. 2B Process C and tagging the neighbor molecules as indicated, the light can be selectively applied to a second (third, fourth, etc.) location in the sample and this process can be repeated as many times as desired. In addition to labeling (depositing labels) to a relatively small number of neighbor molecules in a very small area of a sample, such as due to the use of the microscope analysis to direct the light and the probes described herein, and as explained below, the process can also be performed with sufficiently mild or gentle treatments so that the cell architecture remains intact (e.g., the reactions are also bioorthogonal).

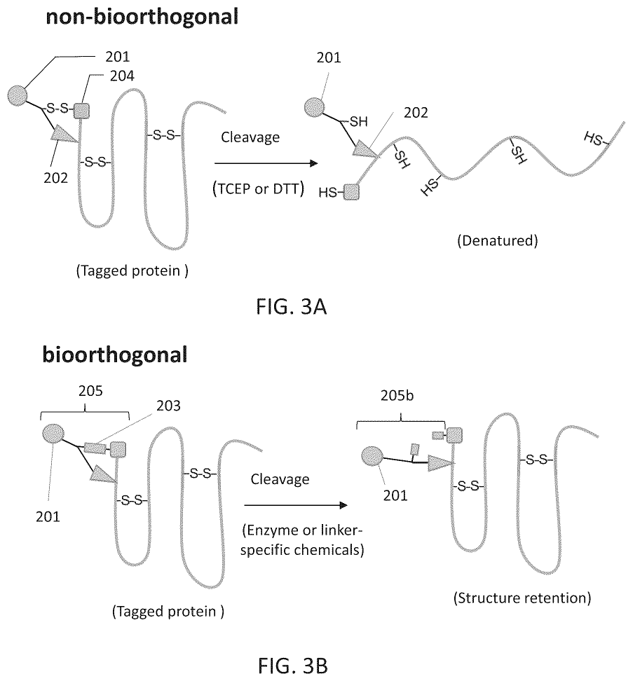

FIG. 3A and FIG. 3B schematically illustrate the effects on protein structure using a multifunctional photoreactive and cleavable probe and mild cleavage conditions as described herein (FIG. 3B), compared with results using a probe with harsh cleavage reactions (FIG. 3A). With mild cleavage conditions the protein structure is retained and the reaction is bioorthogonal, while with harsh cleavage conditions, the protein denatures and the reaction is non-bioorthogonal. FIG. 3A schematically illustrates a relatively harsh cleavage, such as one mediated by use of a reducing agent such as tris (2-carboxyethyl) phosphine (TCEP) or dithiothreitol (DTT) in a cleavage reaction. In addition to cleaving the linker, TCEP or DTT break other disulfide bonds, including naturally occurring covalent disulfide bonds commonly found between cysteine amino acids in proteins, denaturing the proteins. It has been estimated that more than 90% of proteins in cells contain at least one cysteine amino acid and that some one-third of the proteins in the eukaryotic proteome form disulfide bonds. Thus, performing a relatively harsh cleavage on a sample to break disulfide bonds is likely to significantly disrupt protein structure, disrupt overall cell architecture, and alter naturally occurring biomolecule interactions. The cleavage reaction can be considered to be a non-bioorthogonal reaction. In some embodiments, a bioorthogonal reaction preserves structures derived from living organisms (e.g., derived from eukaryotes) and excludes consideration of non-living entity structures, such as viruses.

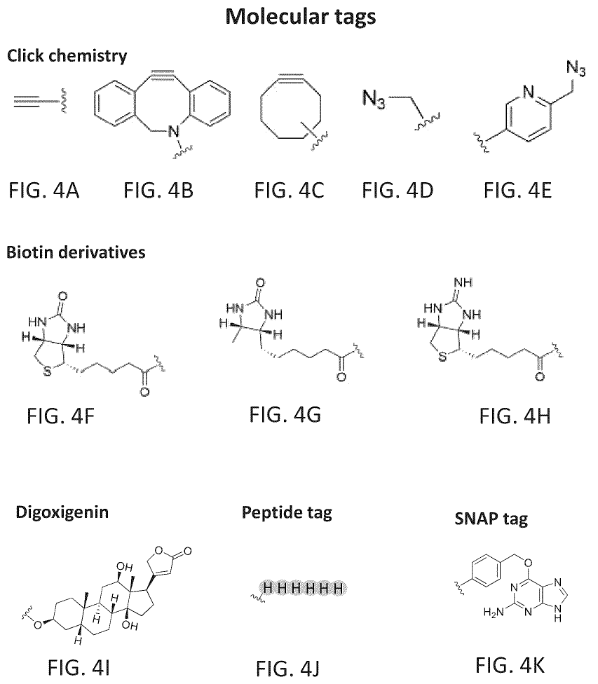

FIG. 4B schematically illustrates a relatively mild cleavage reaction for use with the multifunctional probes described herein. The cleavage reaction uses gentler reagents, such as enzymes or linker-specific chemicals, to cleave the cleavable linker. In some embodiments, mild cleavage reagents are substantially specific. In other words, they substantially and specifically bind to and cleave targets of interest (e.g., the cleavable linker), while substantially not binding to or cleaving other molecules (e.g., less than 1% of the time, less than 0.1%, etc.). In some embodiments, mild cleavage reagents act to cleave other bonds, such as C—C bonds and leave bonds such as disulfide (—S—S—) bonds intact. As illustrated in FIG. 3B, the three-dimensional structure of the protein, mediated by disulfide bonds, remains intact after mild cleavage as described herein. Since tagging and proximity labelling of, for example, naturally occurring neighboring molecules neighboring a protein of interest depends upon the relative proximity of the neighboring molecules to the protein of interest, maintaining the three-dimensional structure of biomolecules and the overall cell architecture can lead to more accurate tagging and labeling of neighboring molecules, reducing both false positives and false negatives in a mild cleavage reaction. The mild cleavage reaction can be bioorthogonal in that it does not substantially disrupt naturally occurring protein structure or cell architecture.

FIGS. 4A-4K show examples of tags that can be used in the photoreactive and cleavable probes described herein. The tags are configured to interact with a detectable label to label biomolecules neighboring a target molecule of interest. FIG. 4A-FIG. 4E show examples of click chemistry tags that can be used with the probes. The click chemistry tag may be, for example, an azide moiety or an alkyne moiety. FIGS. 4F-4H show examples of biotin derivatives that can be used with as probe tags. FIG. 4I shows a digoxigenin moiety tag. FIG. 4J shows a peptide tag. In particular, FIG. 4J shows a poly His tag with 6 histidines (SEQ ID NO:1). However, a histidine tag could instead fewer or more histidines, such as 5 (SEQ ID NO: 33) or 7-10 or more (SEQ ID NO: 34). FIG. 4K shows a SNAP-tag. FIG. 6K shows a SNAP-tag and a CLIP-tag or HaloTag could also be used.

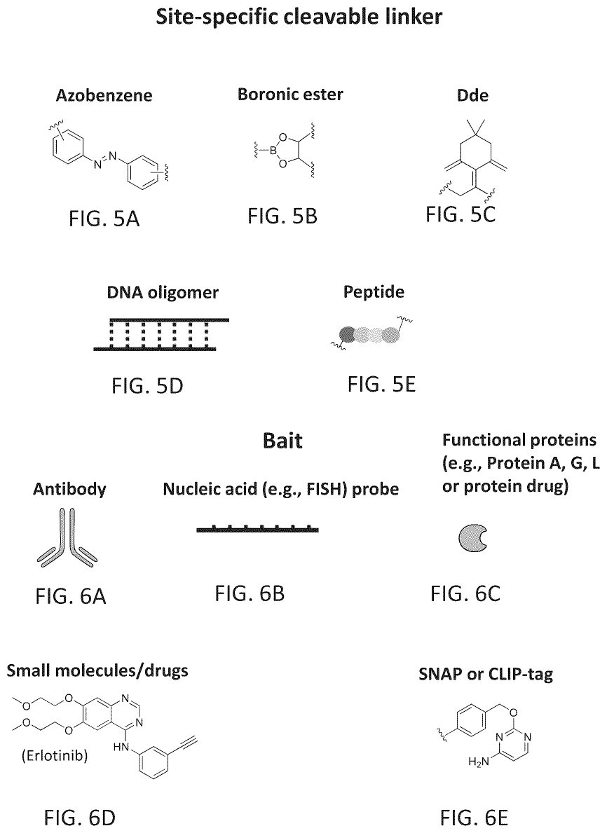

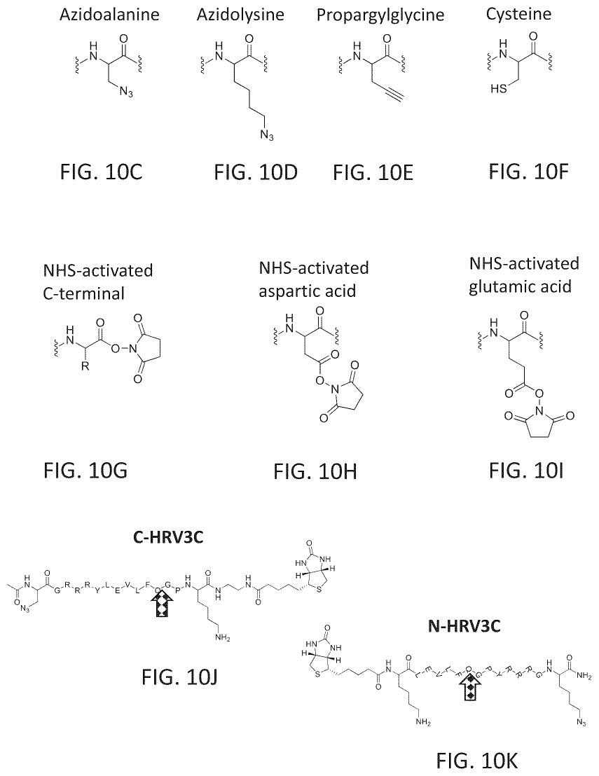

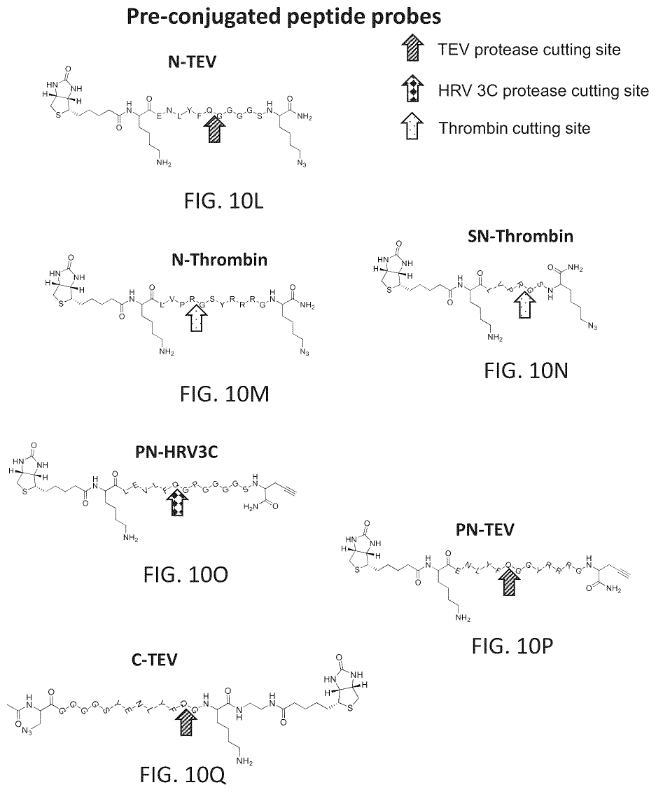

FIGS. 5A-5E show examples of site-specific cleavable linkers that can be used in the photoreactive and cleavable probes described herein. FIG. 5A shows an azobenzene moiety. An azobenzene linker can be cleaved during the cleavage step such as with sodium dithionite or azoreductase. FIG. 5B shows a boronic ester moiety. A boronic ester cleavable linker can be cleaved with thionyl chloride and pyridine. FIG. 5C shows a Dde moiety. The Dde cleavable linker can be cleaved using enzymes or simple small molecules. FIG. 5D shows a DNA oligomer cleavable linker and other nucleic acid molecules can instead be used. DNA oligomers can be cleaved using restriction enzymes, nucleases, or competitive methods using complementary oligomers, depending upon what molecule is labeled. FIG. 5E shows a peptide moiety linker and peptide moiety linkers are discussed below in more detail in reference to FIG. 10A-FIG. 10Q. A peptide linker can be cleaved during the cleavage step using a protease. In some embodiments, a site-specific cleavable linker can be conjugated to a bait molecule. For example, a linker conjugating to bait molecules such as NHS-esters can bind to protein baits, such as antibodies. A particular cleavage linker and associated cleavage reagent can be chosen for various reasons, such as cost or cleavage efficiency.e

FIGS. 6A-6E shows examples of bait molecules that can be used in the photoreactive and cleavable probes described herein to conjugate with a molecule of interest in a sample. FIG. 6A shows an antibody that can be used a bait molecule. Any time of antibody can be used. FIG. 6B shows a nucleic acid portion that can be used as a bait molecule, such as fluorescent in situ hybridization probe (FISH probe). FIG. 6C shows a representation of a functional protein that can be used as a bait molecule. Examples of functional proteins include Protein A, Protein G, Protein L, protein A/G, or a protein drug. Other bait molecules that can be used in the photoreactive and cleavable probes described herein include biologic drugs. Examples of biologic drugs that can be used as bait include abatacept (Orencia); abciximab (ReoPro); abobotulinumtoxinA (Dysport); adalimumab (Humira); adalimumab-atto (Amjevita); ado-trastuzumab emtansine (Kadcyla); aflibercept (Eylea); agalsidase beta (Fabrazyme); albiglutide (Tanzeum); aldesleukin (Proleukin); alemtuzumab (Campath, Lemtrada); alglucosidase alfa (Myozyme, Lumizyme); alirocumab (Praluent); alteplase, cathflo activase (Activase); anakinra (Kineret); asfotase alfa (Strensiq); asparaginase (Elspar); asparaginase erwinia chrysanthemi (Erwinaze); atezolizumab (Tecentriq); basiliximab (Simulect); becaplermin (Regranex); belatacept (Nulojix); belimumab (Benlysta); bevacizumab (Avastin); bezlotoxumab (Zinplava); blinatumomab (Blincyto); brentuximab vedotin (Adcetris); canakinumab (Ilaris); capromab pendetide (ProstaScint); certolizumab pegol (Cimzia); cetuximab (Erbitux); collagenase (Santyl); collagenase clostridium histolyticum (Xiaflex); daclizumab (Zenapax); daclizumab (Zinbryta); daratumumab (Darzalex); darbepoetin alfa (Aranesp); denileukin diftitox (Ontak); denosumab (Prolia, Xgeva); dinutuximab (Unituxin); dornase alfa (Pulmozyme); dulaglutide (Trulicity); ccallantide (Kalbitor); cculizumab (Soliris); elosulfase alfa (Vimizim); clotuzumab (Empliciti); cpoctin alfa (Epogen/Procrit); etanercept (Enbrel); etanercept-szzs (Erelzi); evolocumab (Repatha); filgrastim (Neupogen); filgrastim-sndz (Zarxio); follitropin alpha (Gonal f); galsulfasc (Naglazyme); glucarpidasc (Voraxazc); golimumab (Simponi); golimumab injection (Simponi Aria); ibritumomab tiuxctan (Zevalin); idarucizumab (Praxbind); idursulfasc (Elaprase); incobotulinumtoxinA (Xcomin); infliximab (Remicade); infliximab-dyyb (Inflectra); interferon alfa-2b (Intron A); interferon alfa-n3 (Alferon N Injection); interferon beta-la (Avonex, Rebif); interferon beta-1b (Betaseron, Extavia); interferon gamma-1b (Actimmune); ipilimumab (Yervoy); ixekizumab (Taltz); laronidase (Aldurazyme); mepolizumab (Nucala); methoxy polyethylene glycol-epoctin beta (Mircera); metreleptin (Myalept); natalizumab (Tysabri); necitumumab (Portrazza); nivolumab (Opdivo); obiltoxaximab (Anthim); obinutuzumab (Gazyva); ocriplasmin (Jetrea); ofatumumab (Arzerra); olaratumab (Lartruvo); omalizumab (Xolair); onabotulinumtoxinA (Botox); oprelvekin (Neumega); palifermin (Kepivance); palivizumab (Synagis); panitumumab (Vectibix); parathyroid hormone (Natpara); pegaspargase (Oncaspar); pegfilgrastim (Neulasta); peginterferon alfa-2a (Pegasys); peginterferon alfa-2b (PegIntron, Sylatron); peginterferon beta-1a (Plegridy); pegloticase (Krystexxa); pembrolizumab (Keytruda); pertuzumab (Perjeta); ramucirumab (Cyramza); ranibizumab (Lucentis); rasburicase (Elitek); raxibacumabreslizumab (Cinqair); reteplase (Retavase); rilonacept (Arcalyst); rimabotulinumtoxinB (Myobloc); rituximab (Rituxan); romiplostim (Nplate); sargramostim (Leukine); sebelipase alfa (Kanuma); secukinumab (Cosentyx); siltuximab (Sylvant); tbo-filgrastim (Granix); tenecteplase (TNKase); tocilizumab (Actemra); trastuzumab (Herceptin); ustekinumab (Stelara); vedolizumab (Entyvio); ziv-aflibercept (Zaltrap).

FIG. 6D also shows small molecules/drugs can be used as bait molecules. By way of example, erlotinib is shown. FIG. 6E shows a CLIP-tag and other members of self-labeling moieties could be used (c.g., HaloTag or SNAP-Tag).

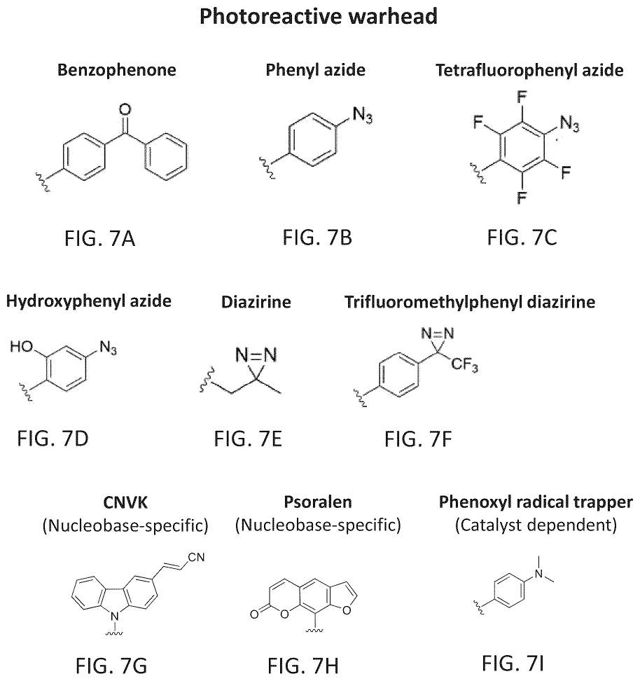

FIGS. 7A-7I show examples of photoactive warheads that can be used in the photoreactive and cleavable probes described herein. FIG. 7A shows a benzophenone photoactive warhead, which can be activated by either 320-365 nm UV-A irradiation of single photon excitation or 720-800 nm of two photon excitation. FIGS. 7B, 7C and 7D shows aryl azide-based warheads which can be activated by either 250-365 nm irradiation of single photon excitation or 800 nm of two photon excitation. FIG. 7B shows phenyl azide photoactive warheads. FIG. 7C shows tetrafluorophenyl azide photoactive warheads. FIG. 7D shows hydroxyphenyl azide photoactive warheads. FIG. 7E shows diazirine photoactive warheads. FIG. 7F shows trifluoromethylphenyl diazirine photoactive warheads. FIG. 7G shows 3-cyanovinylcarbazole nucleoside (CNVK) photoactive warheads which is nucleobase specific. FIG. 7H shows psoralen photoactive warheads which is also nucleobase specific. Psoralens react with DNA or RNA to form covalent adducts. In some embodiments, psoralen photoactive warheads can be activated by long wavelength US light (e.g., UVA, 310-400 nm). FIG. 7I shows phenoxyl radical trapper photoactive warheads which is catalyst dependent. The selection of a particular light-activated warhead can depend on the desired wavelength and the types of the bait molecule. For example, the constituents of the multifunctional probe and constituents for the pre-probe analysis can be chosen so as to not interfere (or minimally interfere) with each other.

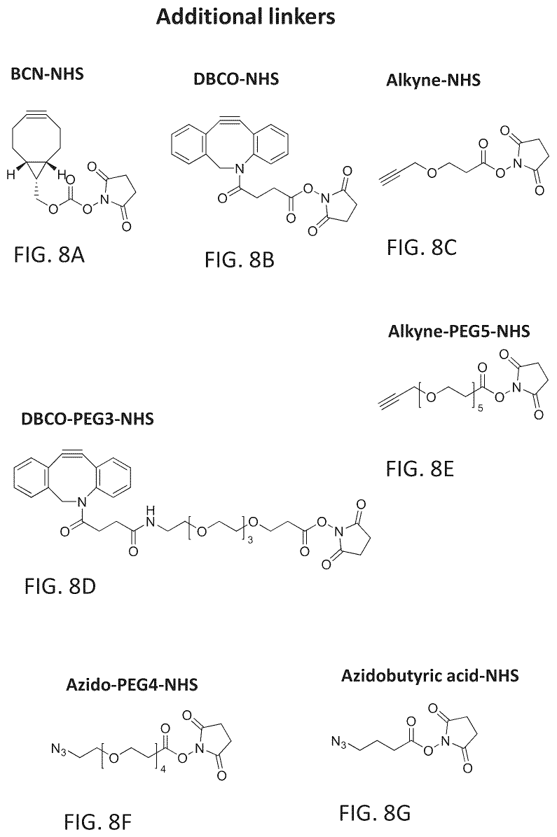

FIG. 8A-8G show additional examples of linkers that can be used as linkers in the photoreactive and cleavable probe described herein. FIG. 8A shows a BCN-NHS linker. FIG. 8B shows DBCO-NHS linker. FIG. 8C shows Alkyne-NHS linker. FIG. 8D shows DBCO-PEG3-NHS linker. FIG. 8E shows Alkyne-PEG5-NHS linker. FIG. 8F shows Azido-PEG4-NHS linker. FIG. 8G shows azidobutyric acid-NHS linker.

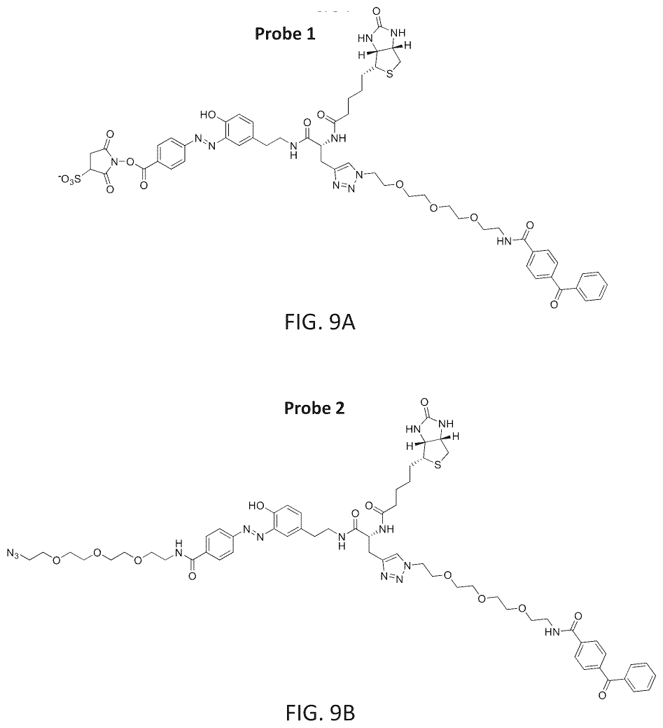

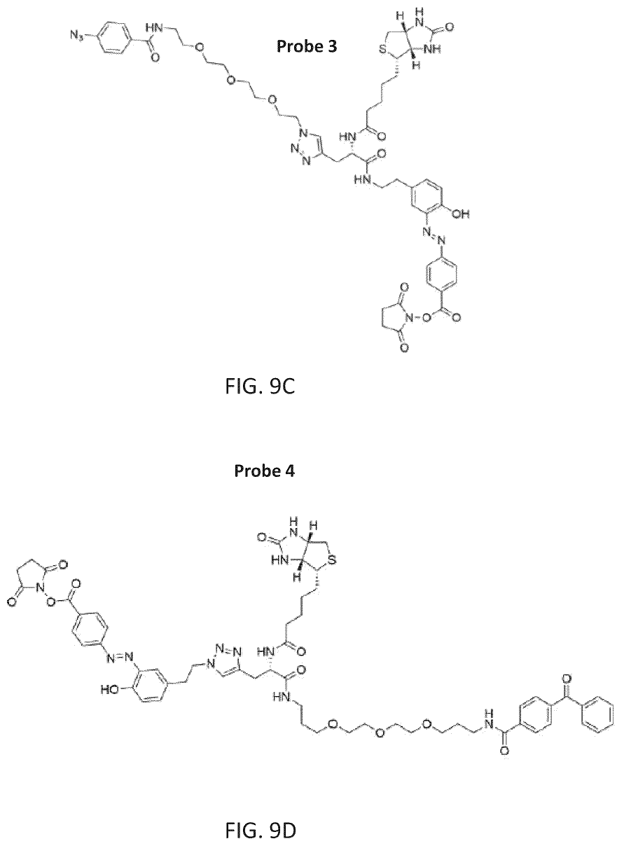

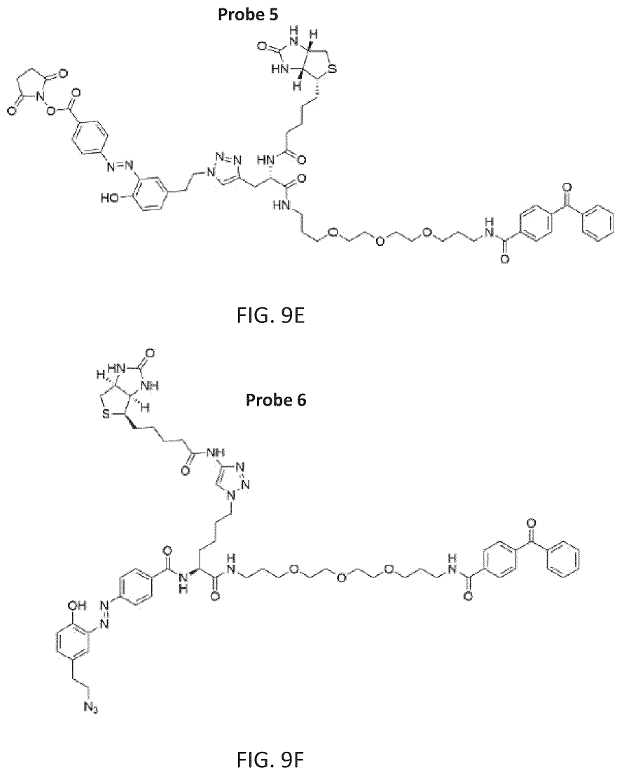

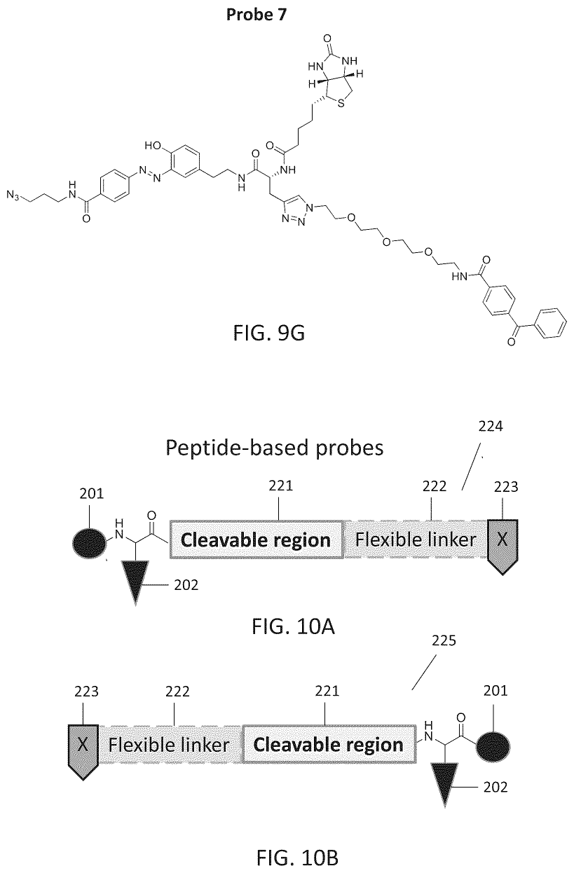

FIGS. 9A-9G show examples of photoreactive and cleavable probes that can be used in the compositions and for practicing the methods described herein. The probes have multivalent cores with a plurality of attachment sites. A tag, a cleavable linker, and a light activated warhead are bound to the attachment sites on the probe. In some embodiments, the multivalent core includes the moiety of formula (I). In some embodiments, n is 1, 2, 3, 4, 5, or 6. In some embodiments, R1 and R2 each independently are hydrogen, substituted, alkyl, substituted alkenyl, substituted alkynyl, substituted carbocyclyl, substituted heterocyclyl, substituted aryl, substituted heteroaryl, or a nitrogen protecting group. In some embodiments one of R3 and R4 is —(CH2)x(OCH2CH2)y(CH2)zNR5R6, and the other is an attachment site, wherein x is 1, 2, 3, 4, 5, or 6; y is 1, 2, 3, 4, 5, or 6; z is 0, 1, 2, 3, 4, 5, or 6; and one of R5 and R6 is an attachment site, and the other is hydrogen, substituted alkyl, substituted alkenyl, substituted alkynyl, substituted carbocyclyl, substituted heterocyclyl, substituted aryl, substituted heteroaryl, or a nitrogen protecting group.

FIGS. 10A-10B schematically illustrate peptide-based photoreactive and cleavable probes. These probes have a peptide region cleavable by a peptide cleavage reagent, such as by a protease that recognizes a specific peptide sequence and the peptide regions are specifically cleavable (e.g., by a protease). FIG. 10A shows an example of a peptide-based probe 224 with tag 201 and photoreactive warhead 202 on the N-terminal end of the peptide region. FIG. 10B shows an example of a peptide-based probe 225 with tag 201 and warhead 202 on the C-terminal end of the peptide region. FIGS. 10A-10B also show probes with an additional, flexible linker 222 (also referred to herein as a spacer) and an optional clickable amino acid 223. FIGS. 10C-10I show examples of reactive or clickable amino acids that can be used with the probes shown in FIGS. 10A and 10B. FIG. 10C shows azidoalanin clickable amino acid. FIG. 10D shows azidolysine clickable amino acid. FIG. 10E shows propargylglycine clickable amino acid. FIG. 10F shows cysteine clickable amino acid. FIG. 10 G shows NHS-activated C-terminal clickable amino acid. FIG. 10 H shows NHS-activated aspartic acid clickable amino acid. FIG. 10 I shows NHS-activated glutamic acid.

FIGS. 10J-10Q show examples of peptide-based photoreactive and cleavable probes schematically illustrated in FIGS. 10A-10B. The cleavage sites for the human rhinovirus 3C (HRV 3C) protease (checkered arrow), tobacco etch virus (TEV) protease (striped arrow), and thrombin (dotted arrow) are indicated. The proteolytically cleavable peptide sequences can be specifically cleaved by a protease during the cleavage step. Examples of proteolytically cleavable peptide sequences that can be used with the probes described herein include those recognized by activated blood coagulation factor X enteropeptidase (also referred to herein as factor X enteropeptidase or factor Xa), human rhinovirus (HRV) 3C protease, thrombin, and tobacco etch virus (TEV) protease. Of these proteases, the factor Xa and thrombin are naturally found in blood. These proteases may recognize and cleave proteins in a cell or cell extract other than the proteolytically cleavable peptide sequences of the peptide-based probes. Although in some cases, this may upset the naturally occurring protein environment in the samples and lead to misleading or artefactual results in some analyses, it is also noted that the number of these cleavage reactions may be sufficiently limited so as to be useful for certain purpose or in certain situations. Cleavage reactions that do not interfere with naturally occurring biomolecules (e.g., naturally occurring proteins in a cell or tissue sample) are considered bioorthogonal and probes cleavable under circumstances that maintain naturally occurring protein structure can be considered to be a bioorthogonally cleavable probe with a bioorthogonally cleavable peptide sequence. As discussed above, while the Sulfo-SBED probe may find use with certain of the methods described herein for particular applications, in other embodiments, the cleavage of the Sulfo-SBED probe with dithiothreitol (DTT) or 2-mercaptoethanol to cleave its S—S bond also undesirably disrupts naturally occurring proteins (e.g., it is non-bioorthogonal).

Enterokinase recognizes the peptide sequence DDDDK| (SEQ ID NO: 2) for cleavage, where cleavage occurs in the linker bond after the lysine.

Factor Xa recognizes the peptide sequence LVPR|GS (SEQ ID NO:3) for cleavage, where cleavage occurs in the linker bond between the arginine and the glycine.

Human rhinovirus (HRV) 3C protease recognizes the peptide sequence LEVLFQ|GP (SEQ ID NO:4) for cleavage, where cleavage occurs in the linker bond between the glutamine and the glycine.

TEV protease prefers the peptide sequence ENLYFQ|S (SEQ ID NO:5) for cleavage, where cleavage occurs in the linker bond between the glutamine and the serine. TEV protease can also recognize the sequence ENLYFQ|G (SEQ ID NO:6) for cleavage, where cleavage occurs between the linker bond between the glutamine and the glycine.

Thrombin recognizes the peptide sequence LVPR|GS (SEQ ID NO:7) for cleavage, where cleavage occurs in the linker bond between the arginine and the glycine

In addition to specific recognition sequences for proteolysis, the peptide portion may contain additional amino acids. Photoreactive and cleavable probes can have either C-terminal or N-terminal tags (e.g., biotinylation).

FIG. 10J shows a C-HRV3C pre-conjugated peptide probe with an HRV 3C proteolytically cleavable peptide sequences GRRRYLEVLFQGP (SEQ ID NO: 8).

FIG. 10K shows an N-HRV3C pre-conjugated peptide probe with an HRV 3C protease cutting site peptide sequence LEVLFQGPYRRRG (SEQ ID NO: 9).

FIG. 10L shows an N-TEV pre-conjugated peptide probe with a TEV protease cutting site peptide sequence ENLYFQGGGGS (SEQ ID NO: 10).

FIG. 10M shows an N-Thrombin pre-conjugated peptide probe with a thrombin protease cutting site peptide sequence LVPRGSYRRRG (SEQ ID NO: 11).

FIG. 10N shows SN-Thrombin conjugated peptide probe with a thrombin protease cutting site peptide sequence LVPRGS (SEQ ID NO: 12).

FIG. 10O shows PN-HRV3C conjugated peptide probe with HRV 3C protease cutting site peptide sequence LEVLFQGPGGGGS (SEQ ID NO: 13).

FIG. 10P shows a PN-TEV conjugated peptide probe with a TEV protease cutting site peptide sequence ENLYFQGGYRRRG (SEQ ID NO: 14).

FIG. 10Q shows a C-TEV conjugated peptide probe with a TEV protease cutting site peptide sequence GGGGSYENLYFQG (SEQ ID NO: 15).

As indicated above, some probes include a flexible linker (also referred to herein as a spacer). Flexible linkers are flexible molecules or stretches of molecules that are used to link two molecules or moieties together. Linkers may be composed of flexible groups so that adjacent domains are free to move relative to another. Flexible linkers may include flexible amino acid residues, such as glycine (G) or serine (S). Flexible linkers may also include threonine (T) and alanine (A) residues. A string of amino acids can be repeated in the linker. For example, a linker may include a length of glycine residues followed by a serine residue, such as forming an (GGGGS)n oligomer, where n is 1, 2, 3, 4, 5, 6, 7, 8 or larger (SEQ ID NO: 16) and the GGGGS motif (SEQ ID NO: 17) is repeated. Flexible linkers can also include alkyl groups, such as a polyethylene glycol (CH₂CH₂O)m linker, where m is from 1 to 50, or 2-30, or 3-6. Other examples of polymeric flexible linkers include polypropylene glycol, polyethylene, polypropylene, polyamides, and polyesters. Flexible linkers can be linear molecules in a chain of at least one or two atoms and can include more.

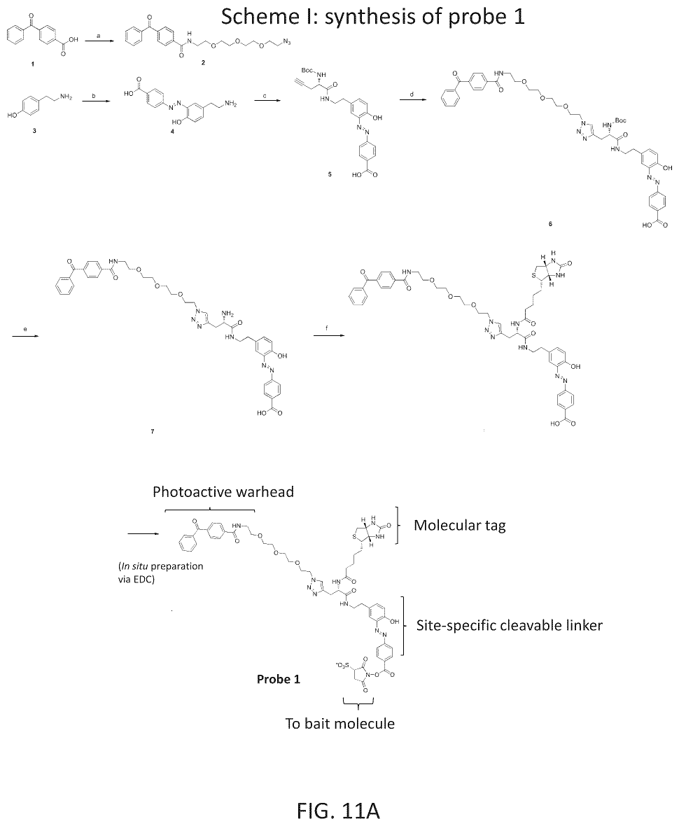

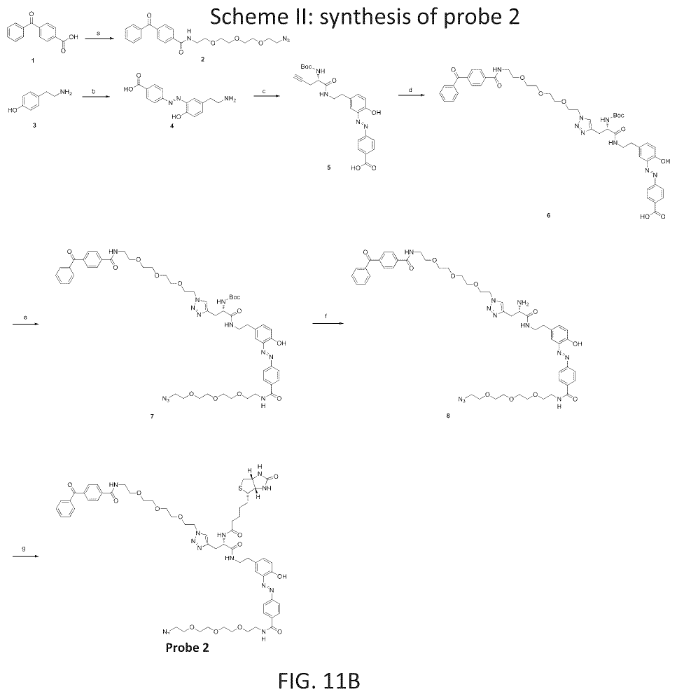

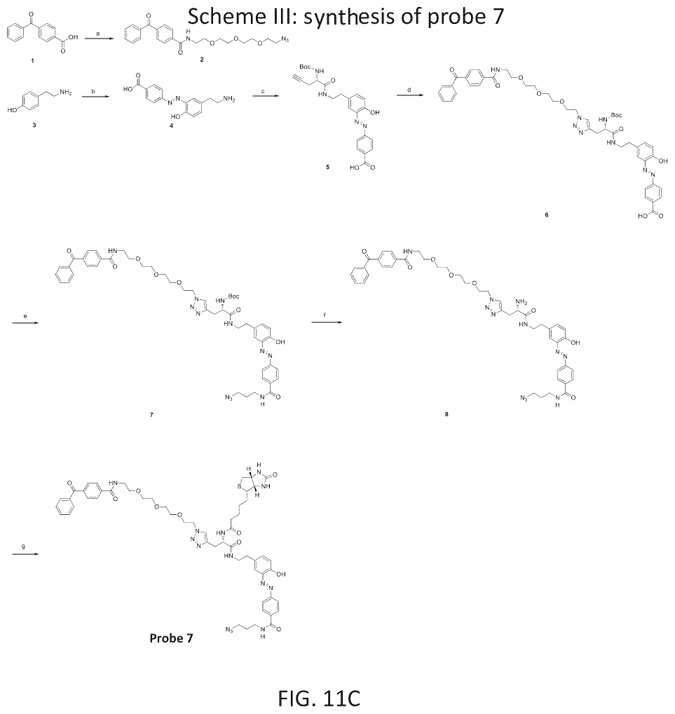

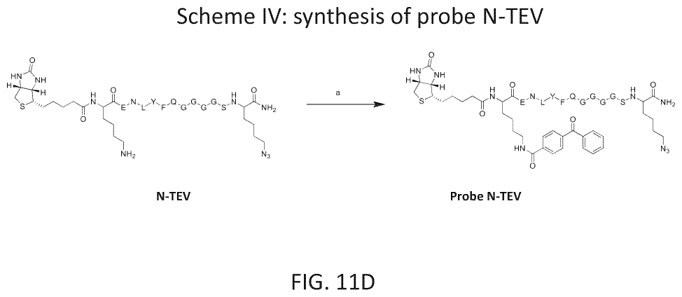

FIGS. 11A-11D illustrate methods to synthesize the photoreactive and cleavable probes described herein. The methods create probes with a tag, a cleavable linker, and a light activated warhead. The figures also illustrate regions where bait molecules can be conjugated. The trifunctional molecular probes can be synthesized by using commercially available molecules as building blocks and regular-used synthesis steps. The schemes shown in FIGS. 11A-11D for synthesis of the probes are given as examples and not for limiting purposes. FIG. 11A shows a synthesis scheme for probe 1. FIG. 11B shows a synthesis scheme for probe 2. FIG. 11C shows a synthesis scheme for probe 7. FIG. 11D shows a synthesis scheme for Probe IV N-TEV.

Some embodiments provide a photoreactive and cleavable probe including a multivalent core comprising a plurality of attachment sites. Some embodiments provide a tag bound to one of the attachment sites, wherein the tag is configured to conjugate to a label. Some embodiments provide a cleavable linker bound to a second of the attachment sites and configured to link to a bait molecule, wherein the cleavable linker includes a peptide sequence. Some embodiments provide a light-activated warhead bound to a third of the attachment sites, wherein the multivalent core includes the moiety of formula (II) or (III):

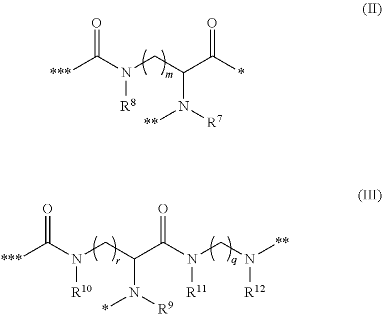

wherein m, r and q each independently are 1, 2, 3, 4, 5, or 6; wherein * comprises an attachment site of one of the plurality of attachment sites for the cleavable linker, wherein ** includes a different attachment site of the plurality of attachment sites for one of either the tag or the photoreactive warhead; wherein includes a different attachment site of the plurality of attachment sites for either the photoreactive warhead or the tag, respectively, and R7, R8, R9, R10, R11, and R12 each independently are hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In some embodiments of the photoreactive and cleavable probe wherein ** includes the attachment site for the tag, and *** includes the attachment site for the photoreactive warhead.

In some embodiments of the photoreactive and cleavable probe, the peptide sequence includes a protease recognition sequence.

In some embodiments of the photoreactive and cleavable probe, wherein the peptide sequence comprises a human rhinovirus 3C (HRV 3C) protease recognition sequence, a tobacco etch virus (TEV) protease recognition sequence, or a thrombin recognition sequence.

In some embodiments of the photoreactive and cleavable probe, the cleavable linker further includes a conjugatable amino acid.

In some embodiments of the photoreactive and cleavable probe, the cleavable linker further includes a cysteine or clickable amino acid amino acid.

In some embodiments of the photoreactive and cleavable probe, the cleavable linker comprises a clickable amino acid with an azido or alkyne moiety.

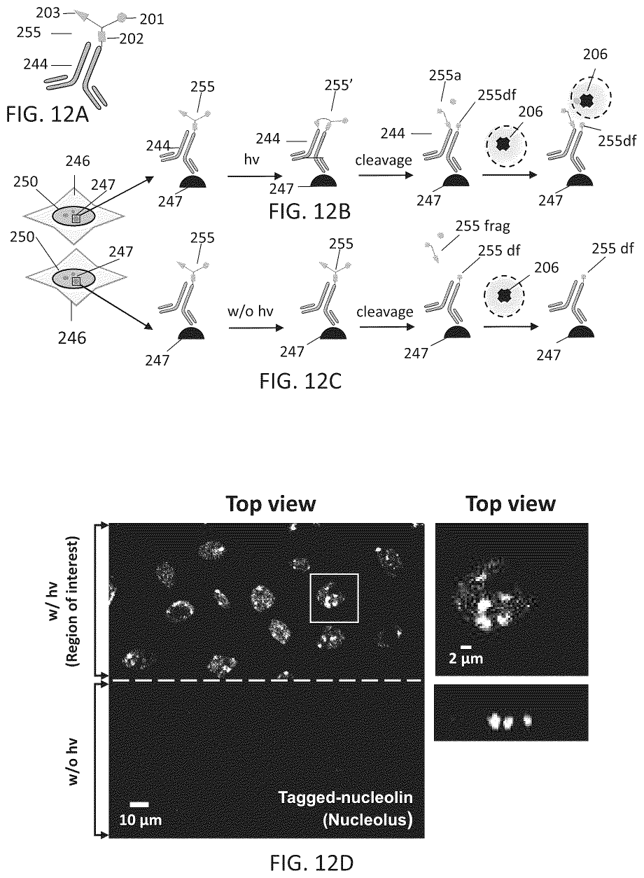

FIG. 12A schematically illustrates a photoreactive and cleavable probe conjugated to an antibody bait. FIG. 12B and FIG. 12C schematically illustrates a reaction scheme for performing photoselective tagging of a molecule using a photoreactive and cleavable probe conjugated to an antibody bait for labeling proteins in the cell nucleolus. FIG. 12B illustrates how the reaction proceeds using controlled light. FIG. 12B also illustrates how the cleavable probes are cleaved to reduce background in non-lighted areas. The reaction shown in FIG. 12B is similar as to that shown in FIG. 2C except that bait molecule 204 is an antibody 244 and the probe 255 includes the antibody 244 as bait. When the photoreactive warhead of the probe 255 is activate, it binds to the bait/antibody 244, rather than a cellular molecule. However, the probe 255 is still retained in the vicinity of the protein of interest, in this case nucleolin in nucleolus 247 in the nucleus 250 of cell 246. During the cleavage step, probe 255 out of the photoselected area is still cleaved into fragment 255 frag and defanged probe fragment 255 df which are washed away during a wash step. FIG. 12D shows results from using the reaction schemes shown in FIG. 12A and FIG. 12B. The nucleolin protein is specifically tagged in the presence of light (top and right panels) but is not tagged in the absence of light (bottom panel). Probes linked to bait molecules are selectively retained through light-activation followed by cleavage and conjugated to enzymes (e.g., HRP in this example) for spatial labeling at a radius from about 10 nm to about 100 nm, depending upon the particular enzymes and reaction times used. In some embodiments of the method, selectively illuminating includes illuminating a zone defined by point spread function.

### Nucleic Acid-Based Probes

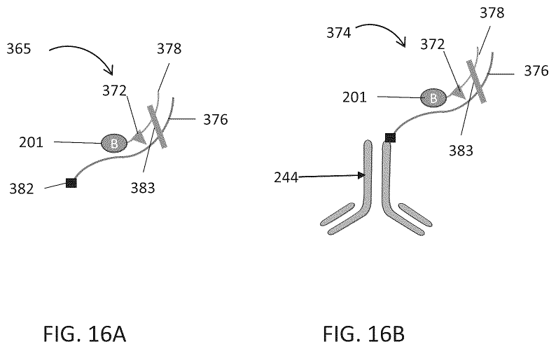

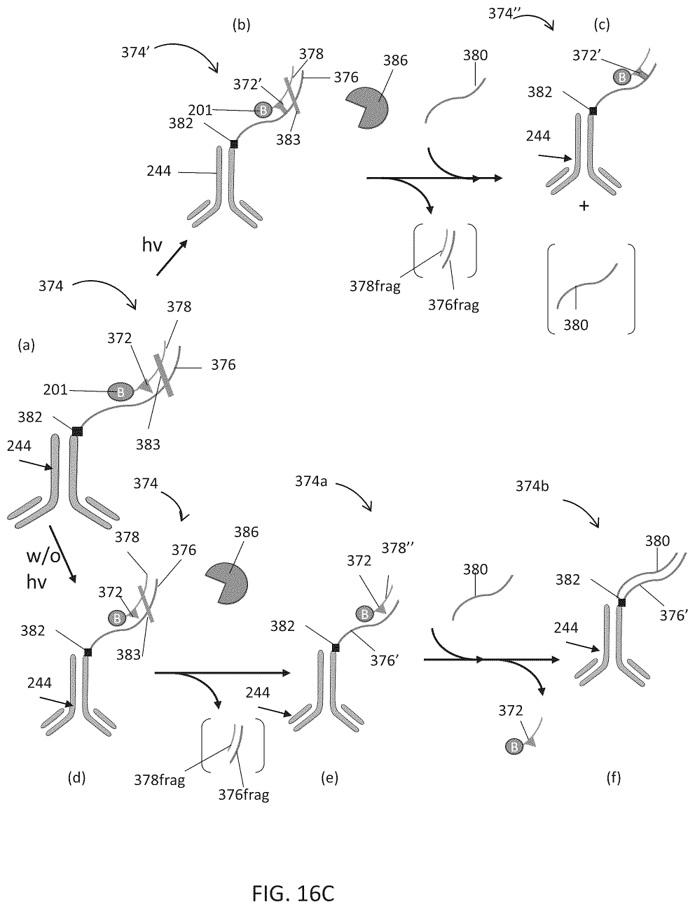

Also provided herein are nucleic acid-based probes with light-activated warheads. These probes may be useful for photoselective tagging and labelling of biomolecules in a biological sample. These probes may use nucleic acid strand displacement to allow selectively labelling of a subset of biomolecules. FIG. 16A shows nucleic acid-based probe 365. Probe 365 includes anchoring strand 376, probing strand 378, tag 201 on probing strand 378 configured to conjugate to a detectable label (including as described elsewhere herein), and bait attachment site 382 on anchoring strand 376. Probing strand 378 is, in part, complementary to anchoring strand 376 and forms a double-stranded structure with anchoring strand 378 along a complementary sequence. An anchoring strand may be continuously complementary to a probing strand and be complementary along an entire length of an anchoring strand or probing strand. In some embodiments, an anchoring strand and a probing strand may have complementary regions of 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides to 15 nucleotides, 16 nucleotides to 20 nucleotides, 21 nucleotides to 25 nucleotides, 26 nucleotides to 30 nucleotides, 31 nucleotides to 40 nucleotides, 41 nucleotides to 50 nucleotides, or 51 nucleotides to 100 n nucleotides. In some embodiments, an anchoring strand and a probing strand may have a plurality of complementary regions separated by non-complementary regions and the total number of complementary nucleotides in the plurality of complementary regions may be at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 45 nucleotides, at least 50 nucleotides, or at least 100 nucleotides. In some embodiments, the total number of complementary nucleotides in the one or plurality of complementary regions may be at least 15 nucleotides or at least 20 nucleotides. An anchoring strand and/or a probing strand can be a nucleic acid (DNA, RNA or both DNA and RNA). FIG. 16A also illustrates cleavable site 383 in the double-stranded structure and cleavable site 383 is configured such that both strands of the probe can be broken or cleaved by a cleaver. A cleaver cleaves a cleavable site. A cleavable site may be, for example, an endonuclease recognition site, such as a restriction endonuclease recognition site with a specific sequence recognized by an enzyme and a cleaver may be a restriction endonuclease, such as a restriction enzyme. FIG. 16A also shows light-activated warhead 372 disposed in probe 365. Light-activated warhead 372 is configured to bond (e.g., covalently bond) anchoring strand 376 to probing strand 378 upon application of light energy which is received by light-activated warhead 372. Light-activated warhead 372 can be, for example, a light-activated nucleobase, such as a thymine specific warhead. Although illustrated in FIG. 16A as part of the probing strand, a light-activated warhead can also or instead be in the anchoring strand, between the strands, etc. Probe 365 can be configured for and can be attached to any type of bait, such as, for example, an antibody, a CLIP-tag, a HaloTag, protein A, protein G, protein L, an RNA molecule, a small molecule, or a SNAP-tag. In some examples, probe 365 can be attached to an antibody, such as a primary antibody or a secondary antibody. FIG. 16B shows probe-bait conjugate 374 with probe 365 attached to antibody 244. A small molecule crosslinker may be used to attach probe 365 to antibody 244. In some variations, a plurality (2, 3, 4, etc.) of individual probes may be attached to a single antibody. The individual probes may be the same as one another or may be different.

FIG. 16C shows an example of probe-bait conjugate 374 (shown in FIG. 16B) in use, such as for selectively labelling part of a biological sample (as shown elsewhere, such as in FIG. 1 and FIG. 2A-FIG. 2C) by selectively illuminating part of the biological sample (to activate the light-activated warhead) and by not illuminating another part of the biological sample (wherein the light-activated warhead is not activated). FIG. 16C illustrates use of competing strand 380 to selectively label biomolecules in light-activated regions. Competing strand 380 is complementary to anchoring strand 376 and, under certain circumstances, competes with probing strand 378 for binding to anchoring strand 376. FIG. 16C depicts probe use when a sample region is illuminated (top) and when a sample region is non-illuminated (bottom). FIG. 16C (top; (b)) depicts light-activated warhead 372′ covalently bonding anchoring strand 376 and probing strand 378. FIG. 16C also depicts adding restriction enzyme 386 to the sample and restriction enzyme 386 cleaving cleavable site 383 (top (c)). Probing strand fragment 378frag and anchoring strand fragment 376frag are no longer attached to the probe or the sample and can be removed (washed away) from the sample. Remaining (and attached to biomolecules such as carbohydrates, lipids, nucleic acids, proteins) are the remainder of the strands, cut probing strand 378′ and cut anchoring strand 376′ in probe-bait conjugate 374″. (Although the cutting of probing strand 378 and anchoring strand 376 is a consequence of the addition of a cleaver (c.g., restriction enzyme) to the biological sample, as explained in detail below and shown in the bottom part of FIG. 16C, the addition of the cleaver (e.g., restriction enzyme) is especially useful for preventing biomolecules in the non-illuminated region of the sample from being tagged). As illustrated in the last panel on the top, competing strand 380 is added to the sample; however, the relatively short cut probing strand 378′ and covalently crosslinked strands at warhead 372′ prevents competing strand 380 from displacing cut probing strand 378′ and competing strand 380 is washed away from light-activated regions. The bottom part of FIG. 16C illustrates how the compositions and processes described herein prevent tagging of biomolecules in non-illuminated regions. In the absence of light, light-activated warhead 372 is not activated and anchoring strand 376 and probing strand 378 are not crosslinked (bottom (d)). Restriction enzyme 386 is added, and (as in the top panel), restriction enzyme 386 cleaves cleavable site 383 (bottom (b to c)). Probing strand fragment 378frag and anchoring strand fragment 376frag are no longer attached to the probe or the sample and can be removed (washed away) from the sample. Probe-bait conjugate 374a is different from probe-bait conjugate 374′ in that the anchoring strand 376′ and probing strand 374′ are not crosslinked. Competing strand 380 is added. In the absence of strand crosslinking, competing strand 380 displaces cut probing strand 378′. This also displaces tag 201 which is attached to cut probing strand 378′. Tag 201 is washed away in this non-illuminated region, leaving only illuminated biological sample regions tagged. These tagged molecules can then be analyzed such as by using an enzyme cascade, NeutrAvidin, etc. or other methods as described herein.

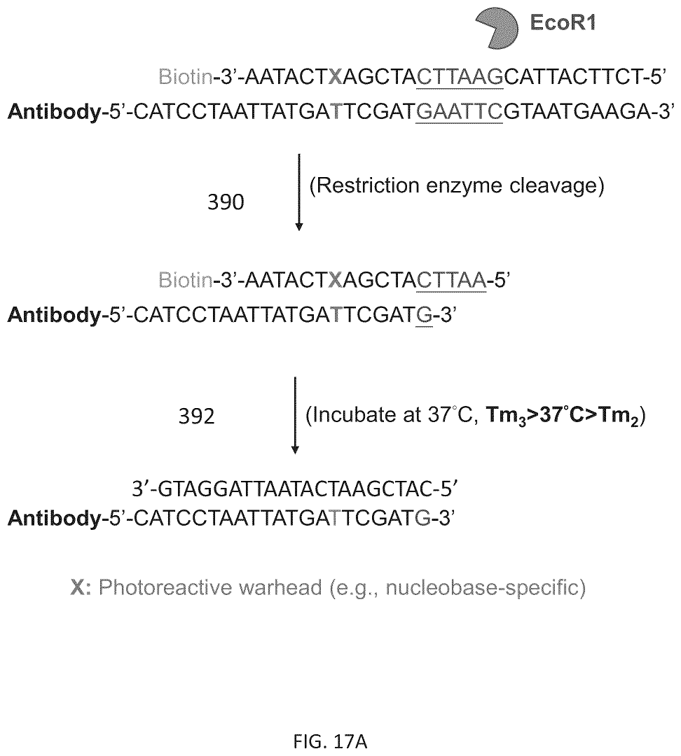

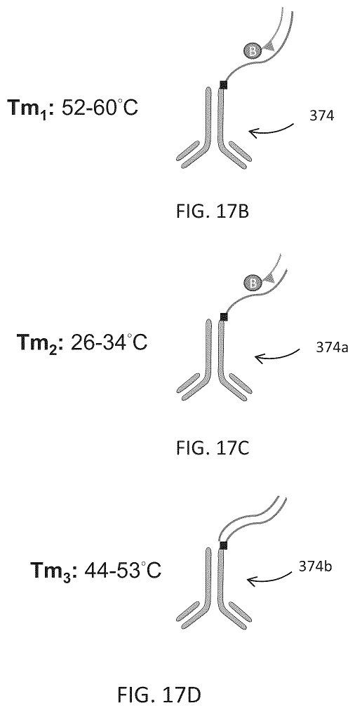

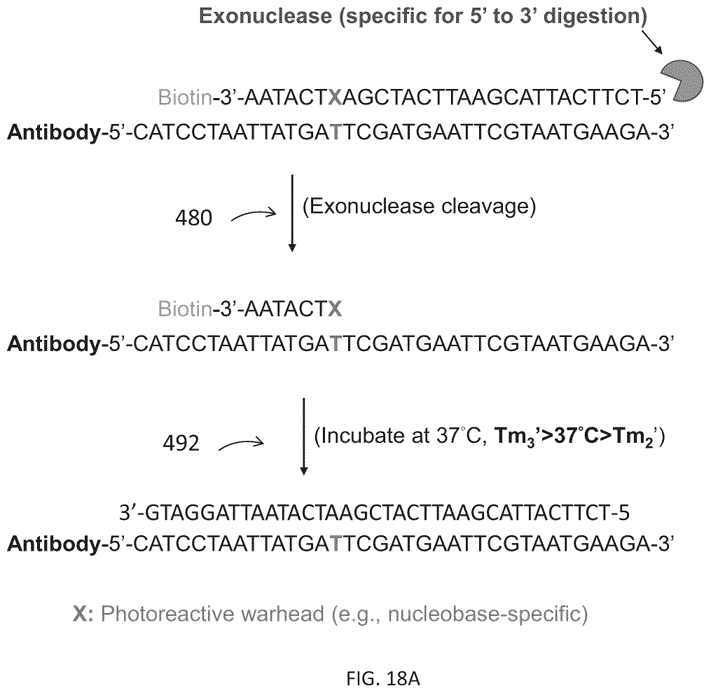

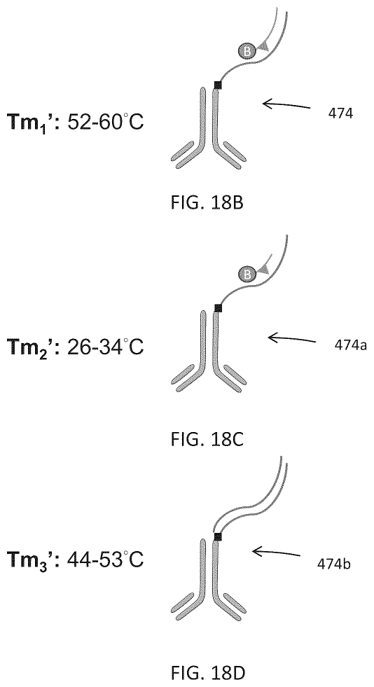

FIGS. 17A-17D illustrate an example probe-bait conjugate and use of a competing strand to remove unwanted tag from some regions of a sample. The top panel of FIG. 17A shows a biotin tagged probing strand (top strand) and anchoring strand (bottom strand) attached to an antibody at the 5′ end of the anchoring strand. This molecule is showing schematically in FIG. 17B. The thymidine specific photoactive warhead (X) is bolded as is its potential thymidine residue binding partner (if the warhead (X) were activated by optical radiation). The restriction enzyme site is underlined. In the region of the sample represented by this example, the region is not exposed to light and the photoreactive warhead (X) does not bind the potential thymidine residue binding partner. With restriction enzyme digestion at step 390, the nucleic acid strands in the probing strand and anchoring strand are cleaved, resulting in shortened probing strand and shortened anchoring strand. This molecule is showing schematically in FIG. 17B. The competing strand is added to the sample and the sample is incubated at step 392 at a desired temperature. The desired temperature (Temp) is higher than the melting temperature Tm₂ of the cleaved probe with the cleaved probing strand and is also lower (Tm_{3>}Temp>Tm₂) than the melting temperature, Tm₃, of probe complex containing the competing strand. The cleaved probing strand (including the tag) is thus removed (denatured or melted away) from the probe. The competing strand becomes hybridized to the anchoring strand. Molecules to which the bait (antibody) are bound thus are not tagged (e.g., in the non-lighted-activated region). The method of any one of claims 27-30 above, wherein a melting temperature Tₘ of the double-stranded structure prior to cleaving the cleavable site is at least 50° C. In some examples, nucleic acid sequences of the probe and/or of the competing strand are sufficiently different from endogenously occurring nucleic acid sequences and are bioorthogonal with them. For example, the sequences of the probe and/or of the competing strand may have no more than 5, no more than 6, no more than 7, no more than 8, no more than 9, no more than 10, no more than 15 nucleotide matches to an endogenously occurring nucleotide sequence in a type of organism of interest (e.g., a eukaryotic sequence, a mammalian sequence, a human sequence, a rat sequence, a mouse sequence, etc.). In some methods herein, a primary sequence of the anchoring strand, a primary sequence of the probing strand, and/or a primary sequence of the competing strand are bioorthogonal with naturally occurring nucleic acid sequences in the biological sample, such that is no sequence matches more than 10 nucleotides in length between endogenous sequences and the probing strands, anchoring strands, or competing strands. Another example for removing a tag from a molecule in a non-light activated region of a sample is illustrated in FIGS. 18A-18D. The probe and method are similar to the probe and methods shown in FIGS. 16A-17D, except that instead of (or in addition) to an endonuclease cleavage, the probing strand is selectively removed from the non-light activated regions using an enzyme or enzyme fragment, followed by strand displacement and replacement with a competing strand. FIG. 18A illustrates photoreactive probe 474 with a photoreactive warhead X (bolded). Probe 474 is similar to probe 374 but lacks a restriction endonuclease cleavage site. Step 480 illustrates an exonuclease, such as the N-terminus fragment of E. coliDNA Polymerase I with 5′ to 3′ exonuclease activity, used to selectively remove the probing strand in a 5′ to 3′ direction. A warhead may inhibit exonuclease activity, and the distances (number of nucleotides) between the tag (biotin) and warhead (X) can be tuned to provide desired melting temperatures of the probes. FIG. 18B schematically illustrates a probe and FIG. 18C and FIG. 18D illustrate altered versions of the probe shown in FIG. 18B and shown in FIG. 18A. The different melting temperatures for the different versions can be used for removing a tag from molecules in part of a sample. For example, to perform the reaction at 37° C., Tm3′>37° C.>Tm2′. Tm1′ can be, for example, from 52° C. to 60°C., Tm2′ can be from 26° C. to 34° C., and Tm3′ can be from 44° C.-53° C.

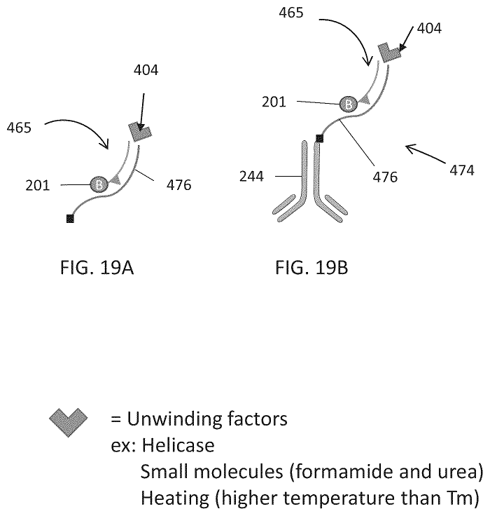

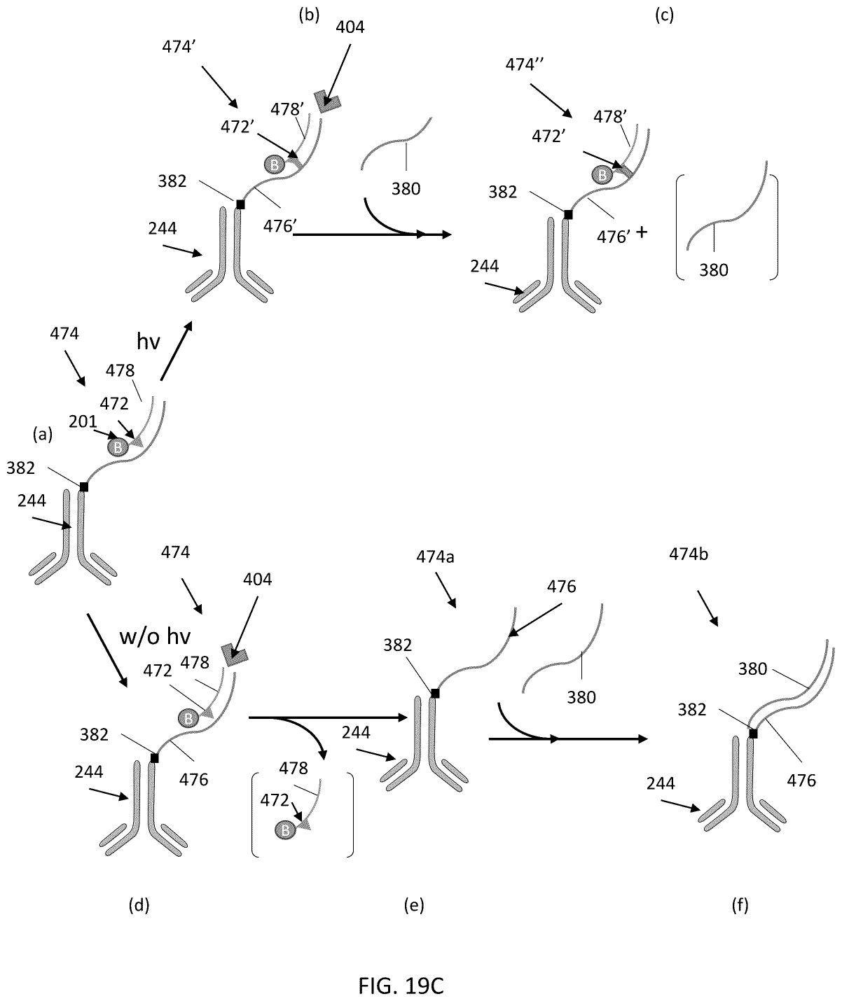

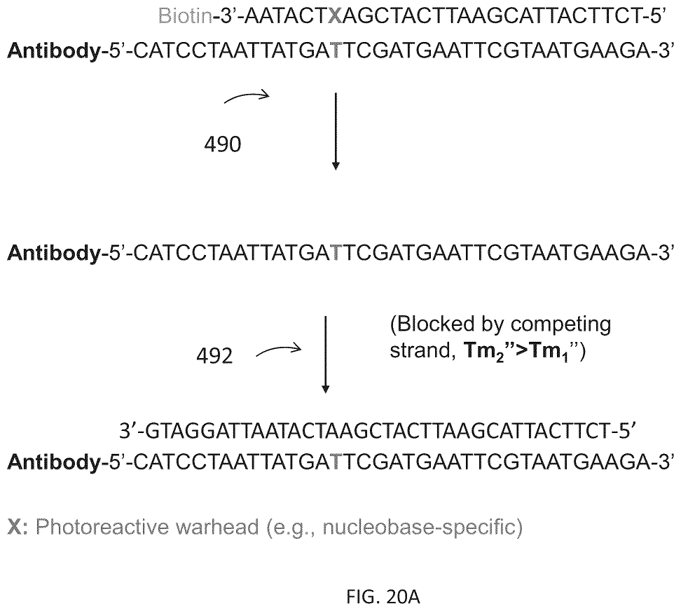

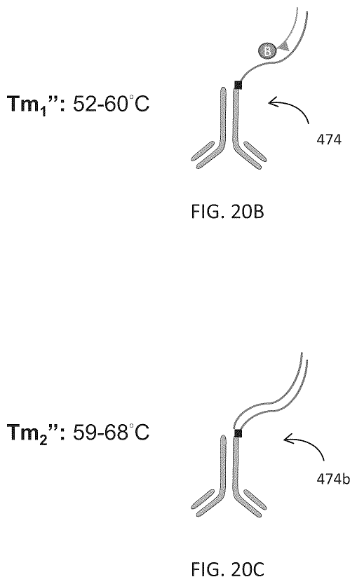

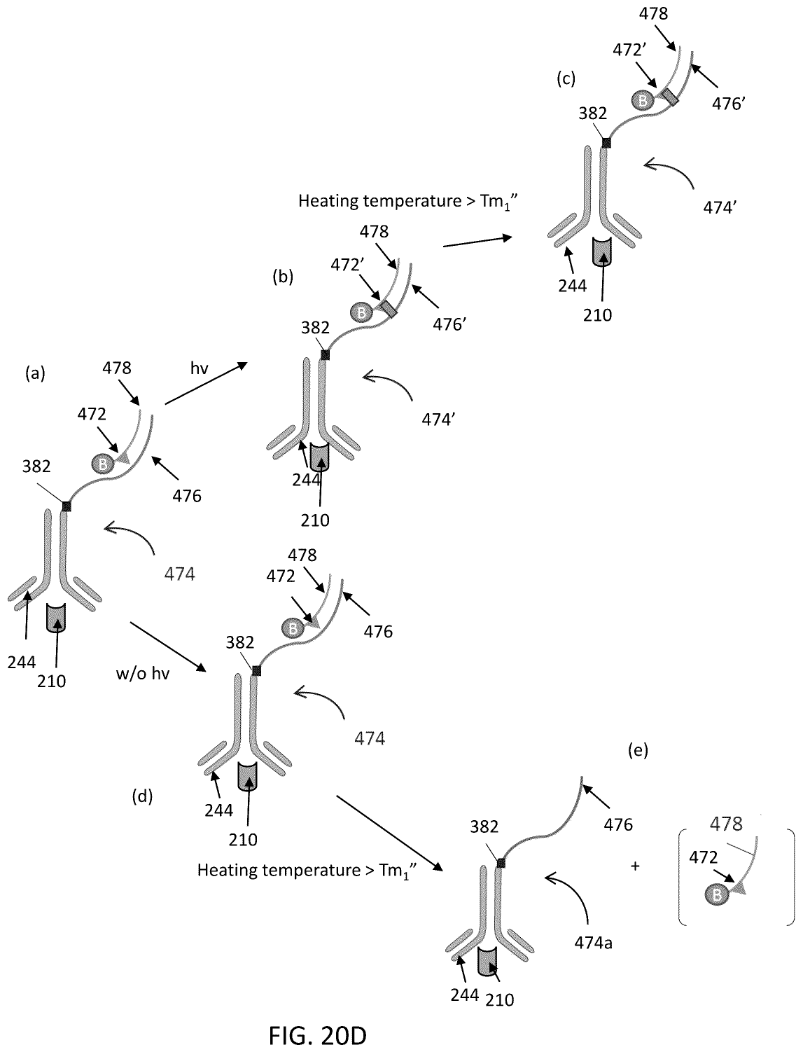

FIGS. 19A-19C and FIGS. 20A-20C schematically also illustrate methods that can be used for removing a tag from molecules in a non-light-activated region of a sample. These probes and methods are similar to the probe and method shown in FIGS. 18A-18D except that an unwinding factor is used to remove the tag rather than an exonuclease and the method can be a cleavage-independent unwinding. FIG. 19A schematically shows probe 465 and unwinding factor 404. FIG. 19A schematically shows probe 465 attached to bait (antibody 244, which can be attached to a molecule of interest, not shown in this FIG). FIG. 19C shows steps in tagging molecules in a light-activated region of a sample and removing a tag from molecules in a non-light-activated region. These steps are similar as to those in FIG. 16C, except that probing strand 478 and anchoring strand 476 lack an endonuclease site. In the top panels (b) and (c), after light activation to activate warhead 472, probing strand 478′ and anchoring strand 476′ are covalently bound at warhead 472. Although unwinding factor 404 is present, the covalently bound probing strand 478′ and anchoring strand 476′ cannot separate and tag 201 remains attached to the bait and molecule of interest. In bottom panels, (d), (e), and (f), warhead 472 is not activated, and unwinding factor 404 is able to unwind (e.g., denature) and separate probing strand 478 from anchoring strand and the rest of the complex. Panel (d) shows unwinding factor 404 beginning to act on probe 474. Panel (c) shows probing strand 478 has been removed from probe 474a. In panel (c), competing strand 380 can bind and prevent probing strand 478 from rehybridizing to probe 474b. An unwinding factor can be an enzyme (e.g., a DNA helicase, an RNA helicase), a small and/or organic molecule (e.g., formamide, urea), heat/temperature treatment, salt treatment, or sodium hydroxide treatment. FIG. 20A-FIG. 20C further illustrate how different melting temperatures for the altered versions of the probe shown in FIG. 20A can be used to facilitate strand displacement and replacement (Step 492) after treatment with an unwinding factor (Step 490). Similar as to described above for FIGS. 17A-18D, the probe 474 with the probing strand (see FIG. 20B) has a melting temperature Tm1″ associated with it (e.g., 52° C.-62° C.) and the probe 474b with the competing strand has a melting temperature Tm2″ associated with it (e.g., 59°C-68° C.). Where Tm2″ is greater than Tm1″, a temperature of the reaction can be utilized to push the reaction to form the probe 474b shown in FIG. 20C.

FIGS. 20D schematically illustrate another method that can be used for removing a tag from molecules in a region of a non-light-activated sample to selectively place the tag on molecules in light activated regions. This probe and method are similar to the probe and method shown in FIG. 19C except that an unwinding factor (e.g., heat is used as an example though an unwinding factor need not be heat) is used to remove the tagged portion of the probe without utilizing a competing strand. FIG. 20D panel (a) schematically shows a biological sample with a photoreactive probe 474. Nucleic acid anchoring strand 476 was delivered to a biological sample and a bait (antibody 244) which can be bound to a molecule of interest 210. After nucleic acid anchoring strand 474 bound to the molecule of interest, unbound anchoring strand was washed away. Next, nucleic acid probing strand 478, which contains a complementary sequence to nucleic acid anchoring strand 476, was delivered and bound to nucleic acid anchoring strand 476 along the complementary sequences to form a double-stranded structure (a photoreactive probe 474). In the top panels (b) and (c), after light activation to activate warhead 472′, probing strand 478′ and anchoring strand 476′ are covalently bound at warhead 472. Although unwinding factor 404 (heat at a temperature above the melting temperature) is delivered to the sample, the covalently bound probing strand 478′ and anchoring strand 476′ cannot separate and tag 201 remains attached to the bait and molecule of interest. In bottom panels, (d), and (c), warhead 472 is not activated, and after application of unwinding factor (heat), the probe unwinds (e.g., denature) and probing strand 478 separates from anchoring strand 476 and the rest of the complex and is washed away. Since the anchoring strand 476 remaining behind on the molecule of interest 210 lacks a tag, molecules of interest 210 in regions not subject to light activation are untagged. In top panels (b) and (c), nucleic acid probing strand is bound to nucleic acid anchoring strand and remains bound, even in the presence of unwinding factors (e.g., heat).

Photoselective tagging and labeling as described herein can be performed in various types of samples, such as samples obtained from tissues, cells, or particles, such as from an entity (e.g., a human subject, a mouse subject, a rat subject, an insect subject, a plant, a fungi, a microorganism, a virus) or tissues samples or cell samples that are not from an organism, such as cell culture samples or artificial tissue scaffold samples (e.g., cultured laboratory cells, in vitro developed heart tissue, 3-d printed tissue, etc.). Samples for analysis using the probes, materials, and methods described herein can be living (live cells) or can be not living (e.g., fixed). A sample for tagging and layering can include a monolayer sample, a multi-layer sample, a sample fixed to a substrate (e.g., a microscope slide), a sample not fixed to a substrate, a suspension of cells, or an extract, such as an in vitro cell extract, a reconstituted cell extract, or a synthetic extract. In some embodiments, a sample is not fixed (unfixed). Examples of probes useful for tagging live cells include those utilizing a small molecule or those sometimes referred to as self-labeling molecules (e.g., Clip-tag, Halo-tag, SNAP-tag). In some embodiments a large number of cells can be automatically analyzed using the methods and materials described herein (e.g., at least about 1,000 cells, at least 10,000 cells, at least 100,000 cells, at least 1 million cells). In some embodiments, a smaller number of cells can be analyzed, such as no more than 1,000 cells, no more than 100 cells, or only a few cells or a single cell. In some embodiments a sample is fixed. For example, a cell or tissue sample may be fixed with e.g., acetic acid, acetone, formaldehyde (4%), formalin (10%), methanol, glutaraldehyde, or picric acid. A fixative may be a relatively strong fixative and may crosslink molecules or may be weaker and not crosslink molecules. A cell or tissue sample for analysis may be frozen, such as using dry ice or flash frozen, prior to analysis. A cell or tissue sample may be embedded in a solid material or semi-solid material such as paraffin or resin prior to analysis. In some embodiments, a cell or tissue sample for analysis may be subject to fixation followed by embedding, such as formalin fixation and paraffin embedding (FFPE).

This disclosure provides an embodiment which is also a microscope-based system for image-guided microscopic illumination. Please refer to FIGS. 14A and 14B. The microscope-based system of this embodiment comprises a microscope 10, an imaging assembly 12, an illuminating assembly 11, and a processing module 13a. The microscope 10 comprises an objective 102 and a stage 101. The stage 101 is configured to be loaded with a sample S. The imaging assembly 12 may comprise a (controllable) camera 121, an imaging light source 122, a focusing device 123, and a first shutter 124. Please further refer to FIG. 14B, the illuminating assembly 11 may comprise an illumination light source 111 and a pattern illumination device 117. The pattern illumination device 117 may include a second shutter 112, a lens module 113 (such as the relay lens 113a and 113b, a quarter wave plate 113c), at least a pair of scanning mirrors 115 and a scan lens 116. Alternatively, digital micromirror device (DMD) or spatial light modulator (SLM) can be used as the pattern illumination device 117.

In this embodiment, the processing module 13a is coupled to the microscope 10, the imaging assembly 12, and the illuminating assembly 11. The processing module 13a can be a computer, a workstation, or a CPU of a computer, which is capable of executing a program designed for operating this system.

The processing module 13a controls the imaging assembly 12 such that the camera 121 acquires at least one image of the sample S of a first field of view, and the image or images are transmitted to the processing module 13a and processed by the processing module 13a automatically in real-time based on a predefined criterion, so as to determine an interested region in the image S and so as to obtain a coordinate information regarding to the interested region. Later, the processing module 13a may control the pattern illumination device 117 of the illuminating assembly 11 to illuminate the interested region of the sample S according to the received coordinate information regarding to the interested region. Also, after the interested region is fully illuminated, the processing module 13a controls the stage 101 of the microscope 10 to move to a second field of view which is subsequent to the first field of view.

In this embodiment, the imaging light source 122 provides an imaging light through an imaging light path to illuminate the sample S during imaging the sample. The first shutter 124, along the imaging light path, is disposed between the image light source 122 and the microscope 10. The controllable camera 121 is disposed on the microscope 10 or on the imaging light path.

Also, the illuminating light source 111 provides an illuminating light through an illuminating light path to illuminate the sample S. The pattern illumination device 117, along the illuminating light path, is disposed between the illumination light source 111 and the microscope 10.

This disclosure provides another embodiment which is also a microscope-based system for image-guided microscopic illumination. This system includes an additional processing module to improve illumination performance and will be describe in detail. Please refer to FIGS. 14A and 14B. FIG. 14A represents a schematic diagram of an imaging-guided system according to one embodiment of the present disclosure, and FIG. 14B depicts the optical path of the image-guided system of FIG. 14A.

As shown in FIGS. 14A and 14B, the microscope-based system 1 for image-guided microscopic illumination comprises a microscope 10, an illuminating assembly 11, an imaging assembly 12, a first processing module 13 and a second processing module 14. The microscope-based system 1 is designed to take a microscope image or images of a sample and use this image or these images to determine and shine an illumination pattern on the sample, finishing all steps for one image rapidly (e.g., within 300 s), and within a short time (e.g., 10 hours) for the entire illumination process for a proteomic study.

The microscope 10 comprises a stage 101, an objective 102 and a subjective 103. The stage is configured to be loaded with a sample S. The stage 101 of the microscope 10 can be a high-precision microscope stage.

The imaging assembly 12 may comprise a camera 121, an imaging light source 122, a focusing device 123, and a first shutter 124. The camera 121 is mounted on the microscope 10. In detail, the camera 121 is coupled to the microscope 10 through the subjective 103 of the microscope 10. The focusing device is coupled to the camera 121 and controlled to facilitate an autofocusing process during imaging of the sample S. The imaging light source 122, which provides an imaging light (as shown in the shaded area in FIG. 14A from imaging assembly 12 to the objective 102) through an imaging light path (as shown with the route indicated by the open arrows in the shaded area depicting the imaging light in FIG. 14A) to illuminate the sample S. The first shutter 124, along the imaging light path, is disposed between the image light source 122 and the microscope 10. The imaging light source 122 can be a tungsten-halogen lamp, an arc lamp, a metal halide lamp, a LED light, a laser, or multiple of them. The shuttering time of the first shutter may vary with the type of the imaging light source 121. Using an LED light source as an example, the shuttering time of the first shutter 124 is 20 microseconds.

If one would like to perform two color imaging, the shutter of the first color light is turned off and the shutter of the second color light is turned on by the first processing module 13. This may take another 40 microseconds. The camera 121 then takes another image with an exposure time of another 20 millisecond. The first processing module 13 then turns off the shutter of the second color light.

In this embodiment, please further refer to FIG. 14B, the illuminating assembly 11 comprises an illuminating light source 111, and a pattern illumination device 117 including a second shutter 112, a lens module 113 (such as the relay lens 113a and 113b, a quarter wave plate 113c), at least a pair of scanning mirrors 115 and a scan lens 116. Alternatively, DMD or SLM can be used as the pattern illumination device 117. The illuminating light source 111 provides an illuminating light (as shown in the open arrows from the illuminating assembly 11 to the objective 102 in FIG. 14A) through an illuminating light path to illuminate the sample S. The second shutter 112, along the illuminating light path, is disposed between the illuminating light source 111 and the microscope 10. The pair of scanning mirrors 115, along the illuminating light path, is disposed between the second shutter 112 and the microscope 10. The camera 121 may be a high-end scientific camera such as an sCMOS or an EMCCD camera with a high quantum efficiency, so that a short exposure time is possible. To get enough photons for image processing, the exposure time is, for example, 20 milliseconds.

The first processing module 13 is coupled to the microscope 10 and the imaging assembly 12. In detail, the first processing module 13 is coupled and therefore controls the camera 121, the imaging light source 122, the first shutter, the focusing device 123, and the stage 101 of the microscope 10, for imaging, focus maintenance, and changes of fields of view. The first processing module 13 can be a computer, a workstation, or a CPU of a computer, which is capable of executing a program designed for operating this system. The first processing module 13 then triggers the camera 121 to take the image of the sample S of a certain field of view (FOV). In addition, the camera 121 can be connected to the first processing module 13 through an USB port or a Camera Link thereon. The controlling and the image-processing procedures of this system will be discussed more detailed in the following paragraphs.

In this embodiment, the second processing module 14 is coupled to the illuminating assembly 11 and the first processing module 13. In detail, the second processing module 14 is coupled to and therefore controls the pattern illumination device 117, including the second shutter 112, and the pair of scanning mirrors, for illuminating the targeted points in the interested region determined by the first processing module 13. The second processing module may be a FPGA, an ASIC board, another CPU, or another computer. The controlling and the image-processing procedures of this system will be discussed more detailed in the following paragraphs.

In brief, the microscope-based system 1 is operated as below. The first processing module 13 controls the imaging assembly 12 such that the camera 121 acquires at least one image of the sample S of a first field of view. The image or images are then transmitted to the first processing module 13 and processed by the first processing module 13 automatically in real-time based on a predefined criterion, so as to determine an interested region in the image and so as to obtain a coordinate information regarding to the interested region. The image processing algorithm is developed independently beforehand using image processing techniques such as thresholding, erosion, filtering, or artificial intelligence trained semantic segmentation methods. Later, the coordinate information regarding to the interested region is transmitted to the second processing module 14. The second processing module 14 controls the illuminating assembly 12 to illuminate the interested region (or, namely, irradiating those targeted points in the interested region) of the sample S according to the received coordinate information regarding to the interested region. In addition, after the interested region is fully illuminated (or all the targeted points in the interested region are irradiated), the first processing module 13 controls the stage 101 of the microscope 10 to move to the next (i.e. the second) field of view which is subsequent to the first field of view. After moving to the subsequent field of view, the method further repeats imaging-image processing-illumination steps, until interested regions of all designated fields of view are illuminated.

Moreover, this disclosure also provides another embodiment which is a microscope-based method for image-guided microscopic illumination. The microscope-based method uses the microscope-based system described above and comprises the following steps (a) to (c): (a) triggering the camera 121 of the imaging assembly 12 by the first processing module 13 to acquire at least one image of the sample S of a first field of view, and the sample S is loaded on the stage 101 of the microscope 10; (b) automatically transmitting the image or images of the sample S to the first processing module 13; (c) based on a predefined criterion, performing image processing of the sample S automatically in real-time by the first processing module 13 to determine an interested region in the image and obtain a coordinate information regarding to the interested region; (d) automatically transmitting the coordinate information regarding to the interested region to the second processing module 14; (e) controlling an illumination assembly 11 by the second processing module 14 according to the received coordinate information to illuminate the interested region in the sample S. Besides, in this embodiment, after the interested region is fully illuminated, the method may further comprise a step of: controlling the stage 101 of the microscope 10 by the first processing module 13 to move to the next (i.e. the second) field of view which is subsequent to the first field of view.

The microscope-based system I used herein are substantially the same as that described above, and the details of the composition and variations of the compositing elements are omitted here.

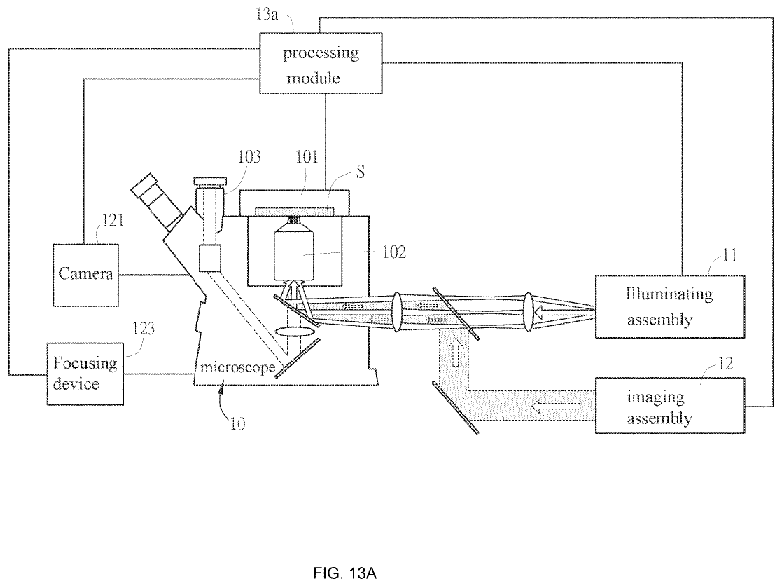

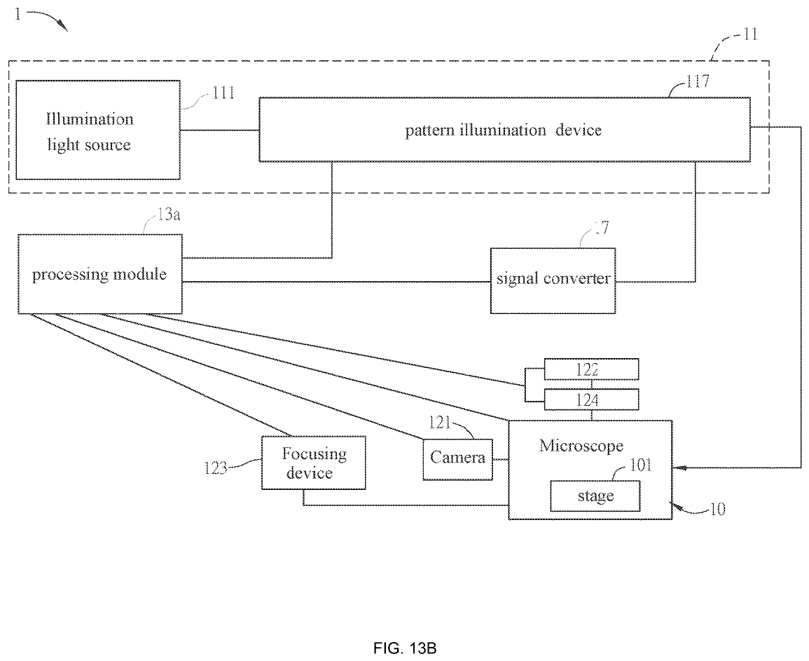

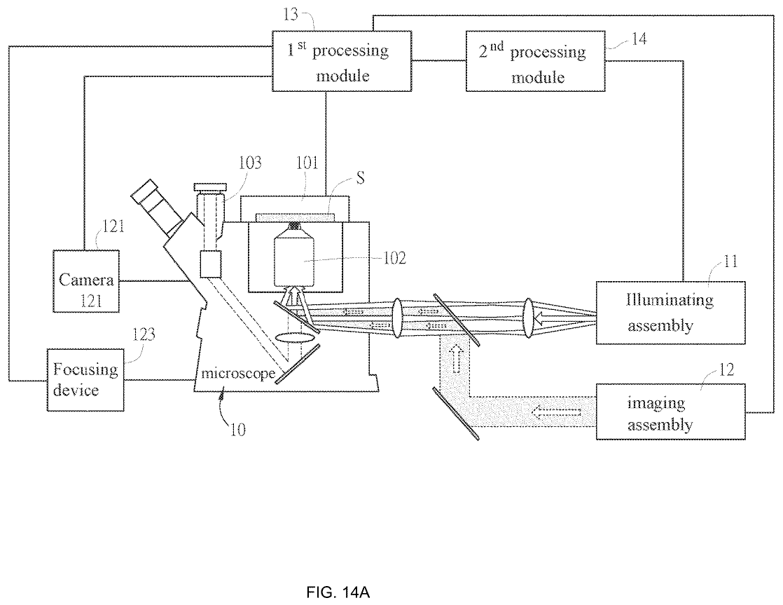

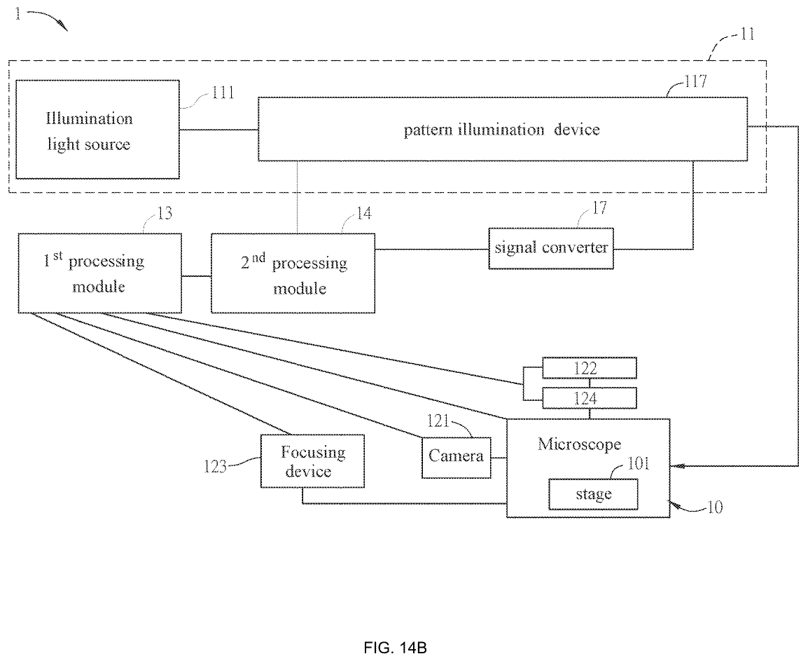

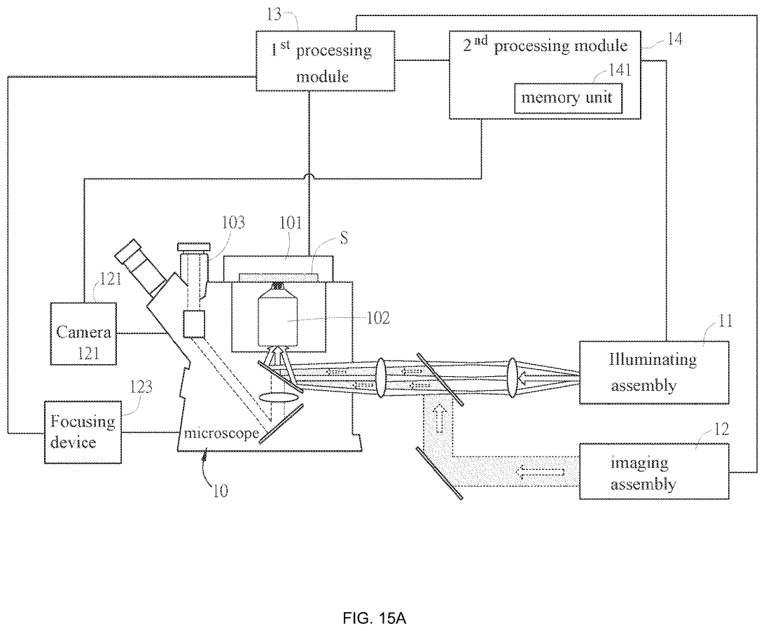

Moreover, as shown in FIGS. 13A and 13B, the light path of the illumination starts from the illumination light source 111. The second shutter 112 is needed for this illumination light source 111. To reach a high switching speed for the point illumination, a mechanical shutter may not be fast enough. One may use an acousto-optic modulator (AOM) or an electro optic modulator (EOM) to achieve the high speed. For example, an AOM can reach 25-nanosecond rise/fall time, enough for the method and system in this embodiment. After the second shutter 112, the beam size may be adjusted by a pair of relay lenses 113a and 113b. After the relay lenses 113a and 113b, the quarter wave plate 113c may facilitate to create circular polarization. The light then reaches the pairs of scanning mirrors (i.e. XY-scanning mirrors) 115 to direct the illumination light to the desired point one at a time. The light then passes a scan lens 116 and a tube lens (included in a microscope, not shown here) and the objective 102 of the microscope 10 to illuminate the targeted point of the sample S. An objective 102 with a high numerical aperture (NA) may be needed to have enough light intensity for photochemical reactions or photoconversion.

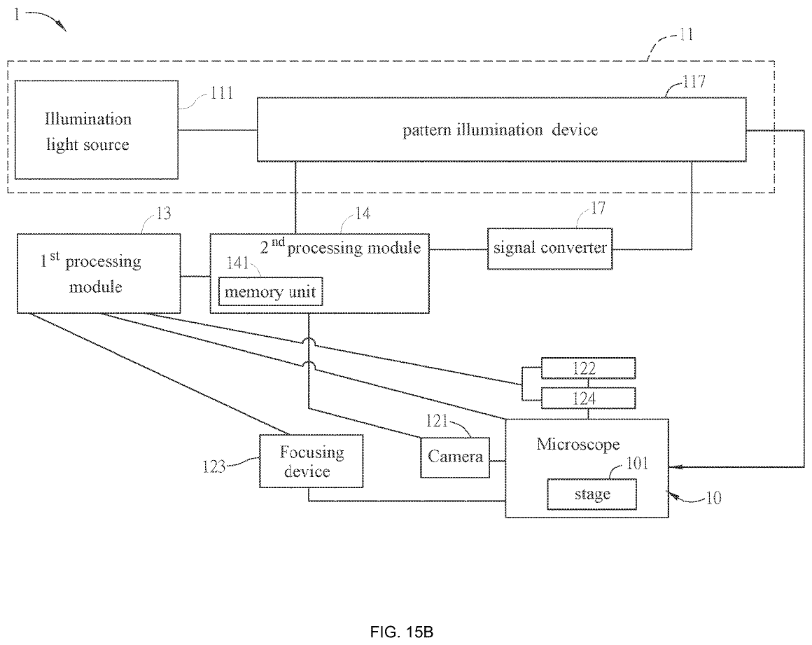

Also, this disclosure also provides another embodiment which is another microscope-based system for image-guided microscopic illumination. The microscope-based system for image-guided microscopic illumination is substantially the same as that is described above. Please refer to FIGS. 15A and 15B. In this embodiment, the microscope-based system 1 comprises a microscope 10, an illuminating assembly 11, an imaging assembly 12, a first processing module 13 and a second processing module 14. The microscope 10 comprises a stage 101, an objective 102 and a subjective 103, and the stage 10 is configured to be loaded with a sample S. Please further refer to both FIG. 15B, the illuminating assembly 11 comprises an illuminating light source 111, and a pattern illumination device 117 including a second shutter 112, at least one relay lens (such as the relay lens 113a and 113b), a quarter wave plate 113c, at least a pair of scanning mirrors 115 and a scan lens 116. Alternatively, DMD or SLM can also be used as the pattern illumination device 117. The imaging assembly 12 may comprise a camera 121, an imaging light source 122, a focusing device 123, and a first shutter 124. The camera 121 is mounted on the microscope 10.

The major difference between the systems described in the previous embodiment and here is that the first processing module 13 here is coupled to the stage 101 of the microscope 10 and the imaging light source 122 and the first shutter 124 of the imaging assembly 12. However, the second processing module 14 here comprises a memory unit 141 and is coupled to the camera 121, the illuminating assembly 11, and the first processing module 13. In other words, in this embodiment, the camera 121 is controlled by the second processing module 14 instead of the first processing module (i.e., the computer) 13. The camera 121 can be connected to the second processing module 14 through a Camera Link if a high speed of image data transfer and processing is required. The memory unit 141 can be a random-access memory (RAM), flash ROM, or a hard drive, and the random access memory may be a dynamic random access memory (DRAM), a static random access Memory (SRAM), or a zero-capacitor random access memory (Z-RAM).

Hence, in the system I embodied here, it is operated as follows. The first processing module 13 controls the imaging assembly 12 and the second processing module controls 14 the camera 121 such that the camera 121 acquires at least one image of the sample S of a first field of view. The image or images are then automatically transmitted to the memory unit 141 to the second processing module 14. Image processing is then performed by the second processing module 14 automatically in real-time based on a predefined criterion, so as to determine an interested region in the image and so as to obtain a coordinate information regarding to the interested region. Later, the second processing module 14 controls the illuminating assembly 11 to illuminate the interested region of the sample S according to the received coordinate information regarding to the interested region.

Because composition, variation or connection relationship to other elements of each detail elements of the microscope-based system I can refer to the previous embodiments, they are not repeated here.

Also, this disclosure also provides still another embodiment which is another microscope-based method for image-guided microscopic illumination. The microscope-based method for image-guided microscopic illumination is substantially the same as that is described above. Please also refer to FIGS. 15A and 15B, the microscope-based method for image-guided microscopic illumination comprises the following steps through (a) to (d): (a) controlling the imaging assembly 12 by the first processing module 13 and triggering the camera 121 of the imaging assembly 12 by the second processing module 14 to acquire at least one image of the sample S of a first field of view, and the sample S is loaded on the stage 101 of the microscope 10; (b) automatically transmitting the image or images of the sample S to the memory unit 141 of the second processing module 14; (c) based on a predefined criterion, performing image processing of the sample S automatically in real-time by the second processing module 14 to determine an interested region in the image and to obtain a coordinate information regarding to the interested region; and (d) controlling the illuminating assembly 11 by the second processing module 14 to illuminate the interested region in the sample S according to the received coordinate information.

The wavelength of light for performing warhead activation or photoselective tagging and labeling ranges in some embodiments from about 200 nm to about 800 nm, e.g., from about 200 nm to about 250 nm, from about 250 nm to about 300 nm, from about 300 nm to about 350 nm, from about 350 nm to about 400 nm, from about 400 nm to about 450 nm, from about 450 nm to about 500 nm, from about 500 nm to about 550 nm, from about 550 nm to about 600 nm, from about 600 nm to about 650 nm, from about 650 nm to about 700 nm, from about 700 nm to about 750 nm, or from about 750 nm to about 800 nm. In some embodiments, the wavelength of light for performing photoselective tagging and labeling is short-wavelength UV light (c.g., 254 nm; 265-275 nm); long-UV light (c.g., 365 nm; 300-460 nm). The wavelength of light for performing warhead activation or photoselective tagging and labeling ranges in some embodiments from about 800 nm to about 2000 nm, e.g., from about 800 nm to about 900 nm, from about 900 nm to about 1000 nm, from about 1000 nm to about 1100 nm, from about 1100 nm to about 1200 nm, from about 1200 nm to about 1300 nm, from about 1300 nm to about 1400 nm, from about 1400 nm to about 1500 nm, from about 1500 nm to about 1600 nm, from about 1600 nm to about 1700 nm, from about 1700 nm to about 1800 nm, from about 1800 nm to about 1900 nm, or from about 1900 nm to about 2000 nm. In some embodiments, the wavelength of light for performing photoselective tagging and labeling is short-wavelength UV light (c.g., 254 nm; 265-275 nm); long-UV light (c.g., 365 nm; 300-460 nm). The wavelengths used for photoactivation of the warhead is different from the wavelengths used for imaging. In some embodiments, photoreactive warhead activation utilizes optical radiation (light) at from around 300-450 nm, 550 nm for single photon activation or >720 nm for multiphoton activation. The particular wavelength depends on the particular warhead. Cleavage can be driven by an enzyme or chemicals (such as sodium dithionite for cleaving azobenzene).

In some embodiments, a multivalent core (c.g., a core moiety) of a probe can be from around 70 Da to about 500 Da. A multivalent core can include or can be a single amino acid or a single nucleotide. In some embodiments, a core can be less than 1 nm in maximal width. Methods

Also disclosed herein are methods of photoselectively tagging and labeling biomolecules and analytical methods. The methods may be used to tag and/or label carbohydrates, lipids, nucleic acids, proteins, either alone or in combination. The methods may include the step of treating a biological sample with a bait molecule and a photoreactive and mildly cleavable probe and binding the bait molecule to a prey in the biological sample. In some embodiments, the probe includes a light-activated warhead and a tag and is bound to the bait molecule through a cleavable linker. Some embodiments include the step of illuminating the biological sample with an imaging lighting source of an image-guided microscope system. Some embodiments include the step of imaging the illuminated sample with a controllable camera. Some embodiments include the step of acquiring with the camera at least one image of subcellular morphology of the sample in a first field of view. Some embodiments include the step of processing the at least one image and determining a region of interest in the sample based on the processed image. Some embodiments include the step of obtaining coordinate information of the region of interest.

Some embodiments include the step of selectively illuminating with a crosslinking light the region of interest based on the obtained coordinate information to thereby doubly crosslink the probe and the bait. Some embodiments include the step of further comprising using the tag to generate a detectable label and labeling proteins proximal the prey with the detectable label. Some embodiments include the step of wherein the detectable label comprises a tyramine label. Some embodiments include the step of, wherein the biological sample comprises a plurality of cells. Some embodiments include the step of wherein the biological sample comprises a plurality of living cells. Some embodiments include the step of wherein the biological sample comprises cell extracts. Some embodiments include the step of wherein selectively illuminating comprises illuminating a region less than 300 nm, less than 200 nm, or less than 100 nm in diameter. Some embodiments include the step of further comprising removing at least the region of interest from the microscope stage.

Some embodiments include the step of further comprising subjecting the sample to mass spectrometry or sequencing analysis. Some embodiments include the wherein the tag comprises a biotin derivative, a click chemistry tag, a HaloTag, a SNAP-tag, a CLIP-tag, digoxigenin, or a peptide tag. Some embodiments include the wherein the click chemistry tag comprises an alkyne-based or azide-based moiety. Some embodiments include the wherein the cleavable linker is an azobenzene derivative, a Dde derivative, a DNA oligomer, a peptide, or a boronic acid ester. Some embodiments include the wherein the bait molecule comprises an antibody, protein A, protein G, protein L, a SNAP-tag, a CLIP-tag or a small molecule. Some embodiments include the wherein the light-activated warhead comprises an aryl azide, a diazirine, or a benzophenone.

Also described herein are photoselective tagging, labeling, and analyzing methods. methods. The methods may include the step of delivering a photoreactive and cleavable probe to a biological sample, wherein the probe comprises a cleavable linker, a light-activated warhead, and a tag and attached to a core of the probe. The methods may include the step of binding a bait molecule to a target biomolecule in the biological sample, wherein the bait molecule is conjugated to the probe. The methods may include the step of illuminating the biological sample from an imaging lighting source of an image-guided microscope system.

The methods may include the step of imaging the illuminated sample with a controllable camera. The methods may include the step of acquiring with the camera at least one image of subcellular morphology of the biological sample in a first field of view. The methods may include the step of processing the at least one image and determining a region of interest in the sample based on the processed image. The methods may include the step of obtaining coordinate information of the region of interest. The methods may include the step of selectively illuminating the region of interest with optical radiation to activate the light-activated warhead and attach the warhead to the target biomolecule or a target biomolecule neighbor such that the probe and target molecule are double-crosslinked. The methods may include the step of cleaving the cleavable linker of the probe. The methods may include the step of removing the cleaved and unbound probe.

Some embodiments include labeling a region less than 300 nm, less than 200 nm, or less than 100 nm in diameter. In some embodiments, the biological sample includes at least one, at least 100, at least 1000 or at least 10,000 live cells.

Some methods include contacting a biological sample having a target biomolecule with a probe as described herein, using optical radiation to spatially selectively photocrosslink the probe with a target biomolecule, cleaving the probe, washing unbound probe or cleaved probe away, labeling the biomolecule/probe complex with a label, and selectively proximity labeling biomolecule neighbor molecules.

Also described are methods for photoactivated labeling including the steps of conjugating any of the photoreactive and cleavable probes (including any nucleic acid probes), to a biomolecule in a biological sample; conjugating a bait molecule to a target biomolecule in the biological sample to crosslink the probe and target biomolecule; delivering optical radiation to activate the light-activated warhead and covalently bond the anchoring strand to the probing strand; cleaving the cleavable site in the double-stranded region of the probe to remove a portion of the anchoring strand and the probing strand away from the rest of the probe; removing the cleaved and unbound probe from the biological sample. In these and other methods herein, a melting temperature Tm of the double-stranded structure prior to cleaving the cleavable site is from 52° C. to 60° C. In these and other methods herein, (i) the probe includes a double-stranded structure after the cleaving step, (ii) the probe includes a double-stranded structure after the replacing step, and (iii) a melting temperature Tm of the double-stranded structure in the second region after cleaving the cleavable site is lower than a melting temperature Tm of the double-stranded structure in the second region after the replacing step in the second region.

In these and other methods herein, the melting temperature Tm of the double-stranded structure in the second region after cleaving the cleavable site is from 26° C. to 34° C. In these and other methods herein, the melting temperature Tm of the double-stranded structure in the second region after the replacing step in the second region is from 44° C. to 53°C. In these and other methods herein, the replacing step is performed at a temperature higher than the melting temperature Tm of the double-stranded structure in the second region after cleaving the cleavable site and lower than the melting temperature Tm of the double-stranded structure in the second region after the replacing step.

Also described are an analytical method including: delivering a photoreactive and cleavable probe to a biological sample, the probe including a nucleic acid anchoring strand and a nucleic acid probing strand, wherein the probing strand forms a double-stranded structure with the anchoring strand along a complementary sequence; selectively illuminating a first region of the biological sample to thereby activate a light-activated warhead disposed in the probe and covalently bond the probing strand to the anchoring strand in the first region, and not illuminating a second portion of the biological sample such that the probing strand and anchoring strand are not covalently bonded in the second region; cleaving a cleavable site in the double-stranded region of the probe to remove a portion of the anchoring strand and the probing strand away from the rest of the probe in the first and second regions; delivering a competing nucleic acid strand to the first and second regions, wherein the competing nucleic acid strand is configured to compete with the probing strand for binding to the anchoring strand; replacing the probing strand with the competing nucleic acid strand in probes in the second region, but not in the first region, wherein the covalent bonding between the probing strand and the anchoring strand in the first region prevents the competing strand from replacing the probing strand.

These methods can further include a tag bound to the probing strand, wherein the tag is configured to conjugate to a detectable label. These methods can further include conjugating a detectable label with the tag and detectably proximity labeling neighbors proximal the target biomolecule by detectable label activity. In some methods, detectably proximity labeling includes photoselectively proximity labeling a region less than 300 nm, less than 200 nm, or less than 100 nm in diameter.

In some methods, wherein a melting temperature Tm of the double-stranded structure prior to cleaving the cleavable site is at least 50° C. In some methods, wherein a melting temperature Tm of the double-stranded structure prior to cleaving the cleavable site is from 52° C. to 60° C. In some methods, wherein the probe includes a double-stranded structure after the cleaving step, wherein a melting temperature Tm of the double-stranded structure in the second region after cleaving the cleavable site is from 52° C. to 60° C. In some methods, wherein the probe includes a double-stranded structure after the replacing step, wherein a melting temperature Tm of the double-stranded structure in the second region after the replacing step in the second region is from 44° C. to 53° C. A melting temperature of a double-stranded structure of a probe (e.g., of an anchoring strand and a probing strand without cleavable site) or of a double-stranded structure that is shorter than a corresponding probe (e.g., a probe cleaved by a cleaver into first and second double-stranded structures) can be from 25° C. to 100° C., such as from 30° C. to 35° C., 35° C. to 40° C., 40° C. to 55° C., 55° C. to 60° C., 60° C. to 65° C., 65° C. to 70° C., including all values between these. Heating a sample to denature a probe or a first or second double-stranded structure can include a step of heating a sample at or above a melting temperature (c.g., at a melting temperature or above a melting temperature by at least 5° C. above a melting temperature, at least 10° C. above a melting temperature, at least 15° C. above a melting temperature, at least 20° C. above a melting temperature, etc.). Heating a sample can include heating above a melting temperature of a probe or a double-stranded structure that is shorter than a corresponding probe and below a melting temperature of another component (such as e.g., a melting temperature of a bait and its target). In some variations, a step of heating a sample can include heating above a melting temperature of a probe or a double-stranded structure that is shorter than a corresponding probe and below a dissociation temperature of another component (such as e.g., a melting temperature of a bait and its target). In these and other embodiments, a temperature or temperature range for heating a sample to remove unwanted probe/tag can be a temperature or a temperature range.

In some methods, wherein a primary sequence of the anchoring strand, a primary sequence of the probing strand, and/or a primary sequence of the competing strand are bioorthogonal with naturally occurring nucleic acids in the biological sample. In some methods, cleaving a cleavable site includes cutting the cleavable site with a restriction endonuclease and/or a restriction enzyme. In some methods, selectively illuminating to activate a light-activated warhead includes activating a nucleobase warhead. In some methods, selectively illuminating to activate a light-activated warhead includes activating a thymine-specific warhead.

In some methods, wherein selectively illuminating the biological sample includes illuminating from an imaging lighting source of an image-guided microscope system, the method further includes imaging the illuminated sample with a controllable camera; acquiring with the camera at least one image of subcellular morphology of the biological sample in a first field of view; processing the at least one image and determining a region of interest in the sample based on the processed image; obtaining coordinate information of the region of interest; These and other methods further include removing cleaved and unbound probe from the first and second regions. In these and other methods, selectively illuminating includes illuminating a region for 25 μs/pixel to 400 μs/pixel, for 50 μs/pixel to 300 μs/pixel, or for 75 μs/pixel to 200 μs/pixel. In these and other methods, selectively illuminating includes illuminating with a power intensity of from 100 mW to 300 mW. In these and other methods, the detectable label includes a catalytic label. In these and other methods, the biological sample includes at least one, at least 100, at least 1000 or at least 10,000 live or fixed cells. In these and other methods, the biological sample includes fixed cells, tissues or cell or tissue extracts. In these and other methods, selectively illuminating includes illuminating a zone defined by point spread function. In these and other methods, the biological sample is disposed on a microscope stage, the method further including removing at least a portion of the illuminated region of the biological sample from the stage. These and other methods include subjecting the sample to mass spectrometry analysis or sequencing analysis. In these and other methods, the tag includes a biotin derivative, a CLIP-tag, a click chemistry tag, digoxigenin, a HaloTag, a peptide tag, or a SNAP-tag.

### Kits

Also provided herein are kits and systems for practicing the methods described herein, e.g., for generating probes, and analyzing, tagging, and labeling biomolecules. Kits will typically include at least one photoreactive and cleavable probe as described herein or components thereof. In some embodiments, the at least one photoreactive and cleavable probe is configured to be mildly cleavable (e.g., bioorthogonally cleavable). In some variations, a kit can include a nucleic acid anchoring strand and/or (e.g., at least one of) an anchoring strand and a probing strand. An anchoring strand can include a bait attachment site. In some variations, a bait attachment site of an anchoring strand is covalently attached to a bait molecule (e.g., an antibody, an RNA probe, etc.). In these and other variations a nucleic acid probing strand, which can be part of a kit, can include wherein the probing strand is configured to form a double-stranded structure with the anchoring strand along a complementary sequence. A light-activated warhead as described herein can be disposed in the probing strand, wherein the light-activated warhead is configured to covalently bound the anchoring strand to the probing strand upon application of light energy. In some variations, a light-activated warhead can be disposed in the anchoring strand instead of or in addition to being disposed in the probing strand. A tag can be bound to the probing strand, wherein the tag is a detectable label. Nucleic acid in a probing strand and an anchoring strand can independently be DNA (i.e., only DNA without RNA), RNA (i.e., RNA without DNA), or both RNA and DNA.

In addition, the kits will typically include instructional materials disclosing means for generating or modifying the one or more probes, such as e.g., attaching a bait moiety to the probe, applying the probe to a sample, conjugating the bait moiety to a prey molecule (in the sample), photocrosslinking the probe via the photoreactive warhead to a molecule of interest, photoreactively cleaving the cleavable linker via the cleavable linker bond, removing (washing away) non-photoreactive probe,

The kits may also include additional components to facilitate the particular application for which the kit is designed. Thus, for example, where a kit contains one or more photoreactive and cleavable probe for tagging and labelling biomolecules, the kit can additionally contain one or more cleavage molecule (e.g., a chemical, an endonuclease, a protease). The kit can additionally contain one or more bait molecules, such as any of those described herein (e.g., an antibody, a functional protein (e.g., protein A, protein G, a protein drug, etc.), a self-labeling protein (e.g., a CLIP-tag, a Halo-Tag, a SNAP-tag), a small molecule or drug.

The kit can additionally contain means of detecting the sample and/or detecting the label (e.g., enzyme substrates for enzymatic labels, filter sets to detect fluorescent labels, enzymes or associated detection reagents, including reagents for performing catalyzed reporter deposition (CARD) or signal amplification (e.g., avidin, Neutravidin, streptavidin, HRP, tyramide, hydrogen peroxide, etc.). The kits may additionally include wash solutions, such as blocking agents, detergents, salts (e.g., sodium chloride, potassium chloride, phosphate buffer saline (PBS)) for one or more steps (e.g., after sample fixation, after probe cleavage, etc.). A kit may include variations of wash solutions, such as concentrates of wash buffers configured to be diluted before use or components to use for making one or more wash solutions) and other reagents routinely used for the practice of a particular method. A kit may include fixatives and other sample preparation materials (e.g., ethanol, methanol, formalin, paraffin, etc.)

The kits can optionally include instructional materials teaching the use of the probes, cleavage molecules, addition of a bait molecule to a probe, and wash solution and the like.

A kit for labeling biomolecules can include any of the photoreactive and cleavable nucleic acid probes described herein, in a first container. A kit may additionally include a competing strand (e.g., strand 380) in a second container, wherein the competing strand is complementary to the anchoring strand. A kit may include a competing strand (e.g., strand 380), wherein when the competing strand and anchoring strand of the kit form a double stranded structure, the structure has a melting temperature Tm of from 44° C. to 53° C., etc., as described elsewhere herein.

A kit can also include an enzyme for removing a probe, such as an endonuclease, an exonuclease, or a helicase. A kit can also include one or more than one unwinding factors, such as a helicase, a small molecule (e.g., formamide, urea), or a salt or base solution.

Also provided herein are kits for probe generation, such as for generation of a nucleic acid probe, such as for generating a probe-bait complex from various baits of interest (such as antibodies, etc.). A kit for probe generation may include a nucleic acid anchoring strand, wherein the anchoring strand comprises a bait attachment site; a nucleic acid probing strand, wherein the probing strand forms a double-stranded structure with the anchoring strand along a complementary sequence; a cleavable site in the double-stranded structure, wherein the anchoring strand and probing strand are configured to break at the cleavable site in response to application of a cleaver; a light-activated warhead, wherein the light-activated warhead is configured to covalently bond the anchoring strand to the probing strand upon application of light energy; and a tag bound to the probing strand, wherein the tag is configured to conjugate to a detectable label.

### Experimental and Methods

Example 1—Demonstration of successful localized photoselective tagging of nucleolin using azo probe. FIG. 12A shows a schematic of photoselective tagging of nucleolin. Nucleolin is a protein found in the nucleolus of eukaryotic cells and involved in the synthesis of ribosomes. Azo-probe 1 was conjugated to a secondary antibody by using BCN-NHS (CAS# 1516551-46-4) as additional linker between Azo-probe 1 and secondary antibody. A sample of U2OS cells was grown on a glass-bottom chamber slide and fixed with 2.4% PFA. The antibody conjugated with Azo-probe I was applied to the sample stained with anti-nucleolin antibody. The sample was exposed to 780 nm two-photon irradiation (200 mW, 200 μs/pixel) to photocrosslink the light activated warhead to the antibody and subsequently incubated with IM sodium dithionite at room temperature for over 16 h to remove non-crosslinked probes. Neutravidin conjugated to Alexa Fluor 647 dye was added and the sample assayed for the Alexa Fluor 647. Alexa Fluor 647 is a bright, far-red-fluorescent dye with excitation ideally suited for the 594 nm or 633 nm laser lines. Results are shown in the top panel of FIG. 12B. A close-up view is shown in the top right side of FIG. 12B. The characteristic nucleoli shape is observed. A side view is shown in the bottom right of FIG. 12B. The bottom of FIG. 12B shows a control region treated the same as in the top panel except that the sample shown in the bottom panel was not exposed to photoactivating light. No significant staining was observed.

### Example 2—Preparation of BCN-Antibody

1. For 100 μl of reaction, 70 μl antibody (1.2-1.5μg/μl) of solution. 2 was prepared. Added 10 μl of 1M sodium bicarbonate (or 1M borate buffer, final 50-100 mM) and BCN-NHS (Sigma-Aldrich #744867, final concentration: 200 μM). Adjusted the final volume to 100 μl with ddH2O. 3. Mixed gently by inverting the tube a few times and mildly spin down. 4. Incubated on shaker/mixer for 1 hour at room temperature. Protected from light if needed. 5. Stopped the reaction by adding 10 μl of 1M glycine and reacted for another 30-60 minutes at room temperature. 6. Removed non-conjugated small molecules by resin filtration using desalting column.

Preparation of Probe 3-antibody conjugate:7. Mixed 0.5-1 μg/μl antibody with probe 3 (final concentration: 100 μM), reacted overnight at 4° C. 8. Removed non-conjugated small molecules by resin filtration using desalting column.

Photoselective labeling: 9. Treated nucleolin-stained cells with Probe 3-antibody and DRAQ5 (nucleus marker) in PBS solution supplement with 0.1% triton for 60 min. 10. Wash the sample with PBS solution supplement with 0.1% Triton and fixed the sample with 2.4% PFA. 11. Defined desired area and labelled the Probe 3-antibody stained nucleolin within the selected area with 160-200 mW pulsed laser at 780 nm. 12. Washed the labeled samples with PBS and incubated with IM sodium dithionite overnight at RT.13. Checked the labeling by staining with NeutrAvidin-Dy550 conjugates (1:200).

### Example 3—Preparation of Probe IV N-TEV

Pre-conjugated peptides N-TEV were dissolved in DMSO/Water (1/1) to 1 mM. N-Succinimidyl 4-Benzoylbenzoate (TCI #S0863) was dissolved in pure anhydrous DMSO to 2 mM. 10 μL of N-TEV stock solution was mixed with 10 μL of N-Succinimidyl 4-Benzoylbenzoate stock solution, 10 μL of 1M sodium borate buffer (pH=8.5), 70 μL of DMSO/Water (1/1) and reacted for 2 h at room temperature. The reaction was quenched by adding 10 μL of IM glycine solution and validated with MALDI-MS.

### Example 4—Preparation of Nucleic Acid Probes

### DNA-Antibody Conjugation

1. Antibodies were purchased from commercial vendors (Jackson Immunoresearch, #111-005-003, Goat anti Rabbit) and initially concentrated to ˜2.5 mg/ml using Amicon Ultra Centrifugal Filters (10 kDa MWCO). Optional: Azide or any other preservatives were removed, and the antibody was buffer-exchanged to phosphate buffered saline (PBS, pH 7.4) using Zeba spin columns (7000 MWCO). For Jackson's antibodies, this storage buffer is already azide/amine free. 2. 200 μg antibody (80 μL) was mixed with 15 μL of 1X PBS and 5 μL of 2.5 mM SM(PEG)2 (PEGylated SMCC) crosslinker (Thermo, #22102) in dimethylformamide (DMF). The molar ratio of SM(PEG)2 to antibody was equal to 9.375. The solution was then incubated at 4° C. for 3 h. 3. Excess PEGylated SM(PEG)2 crosslinker was removed by G-25 columns and concentrated to >2 mg/ml using Amicon Ultra Centrifugal Filters (10 kDa MWCO). 4. In parallel, 130 μL thiol-modified DNA oligos (100 μM, 13 nmole, MW: ˜11 kDa) were mixed with 30 μL of 0.5M dithiothreitol (DTT) and 40 μL of 5X PBS (supplemented with 5 mM EDTA, pH 8.0) for 2 h at room temperature. The reduced DNA oligos were purified using NAP-5 nuclease-free columns (Cytiva/GE Healthcare). Deionized water was used as eluent. 5. Reduced DNA oligos were concentrated to ˜2 μg/μL using Amicon Ultra Centrifugal Filters (3 kDa MWCO). 6. 68 μg (2.72mg/mL, 25 μL) SM(PEG)2 crosslinked (maleimide-activated) antibodies were mixed with ˜136 μg (1.5 μg/μL, 91) of the reduced form of thiol DNA oligos (˜30 eq, Ref: 15 eq) in 1X PBS solution (final volume: 125 μL). The reaction was allowed to proceed for 12 h at 4° C. DNA-antibody conjugates were purified and concentrated using Microcon Centrifugal Filters (30 kDa MWCO).

### Procedures for Sample Preparation

Cells on a chambered coverslip with 4 wells (80427, Ibidi) were fixed with 2.4% paraformaldehyde/PBS for 10 min at RT and permeabilized through incubation with 0.5% Triton X-100 in PBS for 10 min at RT. Cells were then blocked with 3% BSA/0.1% PBST for 1 h, then treated with 0.002% streptavidin in 0.1% PBST for 30 min at RT to block the endogenous biotin. After three times of 0.1% PBST washes, 40 μM biotin was added into the wells for blocking residual streptavidin.

Immunochemistry and in situ hybridization

Cells were stained with appropriate concentration (normally 1-10 μg/mL) of primary antibody of desired target in 3% BSA/0.1% PBST for 1 h at RT. DNA-conjugated secondary antibody was prepared to 10 μg/mL in 3% BSA/0.1% PBST or hybridization buffer (2x saline-sodium citrate (SSC) buffer, 10% dextran sulfate, 1 mg/mL yeast tRNA, 5% normal donkey scrum or BSA) and was incubated with the sample for 1 hr at room temperature or at 4° C. overnight, for binding primary antibody. Biotin-tagged/warhead-bound oligo probe was prepared to 0.5-1 μg/mL in 0.1% PBST or hybridization buffer and was incubated with the sample for 1 hr at room temperature or at 4° C. overnight, to hybridized with the DNA-antibody conjugates.

### Photocrosslinking of Hybridized dsDNA-Antibody Conjugate

Another secondary antibody or oligo probe with fluorophore was used to visualize target protein (if needed) for choosing ROI and covalent photocrosslinking of hybridized strands.

### Removal of Probing Oligo

Sample was incubated with digestion buffer (ex: CutSmart® Buffer) containing sequence-specific restriction enzyme and a competing strand for replacement of digested DNA-antibody conjugates. Reacted for 60 min to overnight at 37° C. Washed three times with 0.1% PBST. (Other nucleases with specific reactivity are also applicable, such as 5′->3′ exonuclease or 3′->5′ exonuclease, which depends on the attaching site of anchoring DNA to antibody.)

### Validation of Light-Driven Tag Capturing

Neutravidin-fluorophore conjugate (1 μg/mL in 3% BSA/PBST, 1 h) was used to probe the biotin-labeled region after illumination.

### Example 5—Preparation of NHS-(PEG)2-maleimide-Ab Conjugation

1. Antibodies were purchased from commercial vendors (Jackson Immunoresearch, Dk anti Ms) and initially concentrated to 2.5 to 3 mg/ml using 30 kDa filters. 2. Appropriate amounts of concentrated antibodies were mixed with 2.50 mM NHS-(PEG)2-maleimide crosslinker (Thermofisher, #22102) dissolved in DMF and stored at −20° C. for around 30 days (about 4 and a half weeks) with molar ratio of NHS-(PEG)2-maleimide to antibody being equal to 12 in PBS (pH=7.2˜7.4). The concentration of antibody was adjusted with PBS until it was equal to 2 to 3 mg/ml). 3. The reaction was then incubated at 4° C. for 3 h. 4. Excess NHS-(PEG)2-maleimide crosslinkers were removed by G-25 columns and concentrated to >2 mg/ml by using Amicon Ultra Centrifugal Filters (30 kDa MWCO)

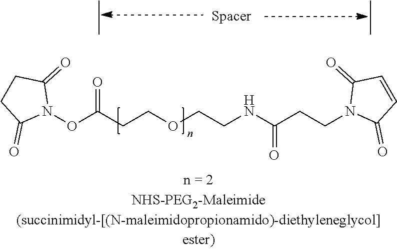

### NHS-(PEG)2-maleimide Conjugated Antibody Reacts with Reduced thiol-DNA

1. Appropriate volume of NHS-(PEG)2-maleimide conjugated antibody (2.5˜3 mg/mL in 1X PBS) was mixed with dried thiol-DNA oligos (reduced form) with the molar ratio equal to 1:25 (antibody: DNA) by adding the antibody solution into the tube containing lyophilized DNA. 2. The reaction was mixed by vortexing for a few minutes and centrifuged at >10,000 RCF. 3. The reaction was allowed to proceed for >12 h at 4° C. 4. DNA-antibody conjugates were purified and concentrated using Microcon Centrifugal Filters (100 kDa MWCO).

### Example 6—dsDNA-Antibody Conjugate Enables the Enrichment of Protein Constituents of Arsenite-Induced Stress Granules

Stress granules (SGs) are dynamic ribonucleoprotein assemblies that are formed under cellular stress. Given their pathological implications in neoplastic and neurodegenerative processes, characterization of SG composition is crucial for therapeutic explorations. However, purification and enrichment of SGs has been a major challenge in the field due to the diminutive, membrane-less structure of SGs. Here, SGs were induced in U2-OS cells via arsenite exposure and stained with SG marker G3BP1.

### Cell Preparation

U2-OS cells were fixed with PFA or PFA/GA. Permeabilized fixed cells were permeabilized by incubating with 0.1 to 0.5% PBST for 10 minutes, and then washed 3 times with 0.1% PBST. Cells were incubated with blocking buffer (3% BSA) for 1 hour, with gentle shaking, then washed twice with 0.1% PBST. Following this, (to reduce background) cells were incubated with 0.002% streptavidin (in 0.1% PBST) for 30 minutes, and then washed twice with 0.1% PBST. Finally, the cells were incubated with 40 μM biotin (in 0.1% PBST) for 15 minutes, and then washed 3 times with 0.1% PBST.

### Hybridization with dsDNA-Antibody Conjugate

1. Used general immunofluorescence (IF) protocol until 1^{st} Antibody treatment and then washed three times with PBST.
2. Incubated with hybridization blocking buffer (freshly prepared) for 60 min at RT with 30 μL per coverslip and 250 μL per well of 2 well plate. If nucleus background was high, dextran sulfate could have been used at a final concentration 0.02% to 0.5%. (stock: 5% dextran sulfate in DEPC H₂O). The components of hybridization blocking buffer is provided below in Table 1.

TABLE 1

| Reagent | Volume | | Note |
|---|---|---|---|
| 10% BSA | 50 | μL | in DEPC H₂O |
| 10% Triton-X 100 | 1 | μL | in DEPC H₂O |
| 10X PBS | 10 | μL | Cell culture level or |
| | | | autoclaved |
| Salmon sperm DNA | 10 | μL | |
| DEPC H₂O | 29 | μL | |
| Total | 100 | μL | |

3. Incubated with 10 μg/mL Ab-DNA in hybridization blocking buffer for 60 min at RT. Ab is donkey anti-mouse IgG. DNA sequence is shown in FIG. 20A (5′ to 3′). The DNA sequence is the same from 5′ to 3′as shown in FIG. 20A. Washed three times with PBST. If nucleus background was high, dextran sulfate could have been used at a final concentration 0.02% to 0.5%. (stock: 5% dextran sulfate in DEPC H₂O). The components of hybridization blocking buffer added with Ab-dsDNA is provided below in Table 2.

TABLE 2

| Reagent | Volume | | Note |
|---|---|---|---|
| 10% BSA | 50 | μL | in DEPC H₂O |
| 10% Triton-X 100 | 1 | μL | in DEPC H₂O |
| 10X PBS | 10 | μL | Cell culture level or |
| | | | autoclaved |
| Salmon sperm DNA | 10 | μL | |
| Ab-DNA | X μL (for 1/25 dilute, X = 4) | | |
| DEPC H₂O | 29-X | μL | |
| Total | 100 | μL | |

4. Incubated with 1 μM biotin-tagged DNA probing strand (with or without warhead) and 3^{rd} Ab (for Dk Ab, used 1:200) in PBST supplement with 3% BSA for 60 min at RT. DNA sequence of the probing strand is shown in FIG. 20A (3′ to 5′). The third antibody is Rabbit anti-Donkey IgG (H+L) Secondary Antibody, DyLight™ 650, which is used to confirm region of interest (ROIs).
5. Washed three times with PBST and another three times with PBS.

### Photocrosslinking of hybridized dsDNA-antibody conjugate

After hybridization of dsDNA-antibody conjugate, one can confirm ROIs (stress granule) by fluorescence microscope. The entire ROIs of U2-OS cells were illuminated with a 780-nm femtosecond laser to activate the light-activated warhead disposed in the probing strand and covalently bind the probing strand to the anchoring strand.

### Dehybridization

1. Used wet tissue paper to cover the surface of heating plate. 2. Set the heating plate and heated PBS (PH˜7) and ddH₂O to 70 to 75 degrees. Hot ddH₂O was used for keeping the tissue paper moist. 3. Washed the sample with pre-heated PBS for four times (3 min per wash) and another 2 times with PBST at RT. 4. Continued with Dy-488-Neutravidin staining (usually 1:500).

### Amplification of Biotin Signal of Probing Strand

1. Incubated cells with 1/1000 Biotin-HRP (3% BSA in PBST or PBS) for 30 minutes. 2. Washed with PBS and discarded immediately. Then washed with PBS again, exchanged 5 times, exchanging wash buffer every 10˜12 minutes. 3. Mixed BP and H₂O₂ to 500 uM BP and 0.012% H₂O₂ and incubated cells for 3 minutes. 4. Prepared 2X and 1X amp quencher. 2X amp quencher: 0.04% NaN₃, 20 mM sodium ascorbate (SA) and 10 mM Trolox (Tx) in PBS. 1X amp quencher: 0.02% NaN₃, 10 mM sodium ascorbate (SA) and 5 mM Trolox (Tx) in PBST. 5. Added equal amount of 2X amp quencher directly and then removed. 6. Washed 3 times with 1X amp quencher. 7. Washed 3 times with PBS. 8. After amplification, sample was subject to LC-MS/MS analysis.

### Results

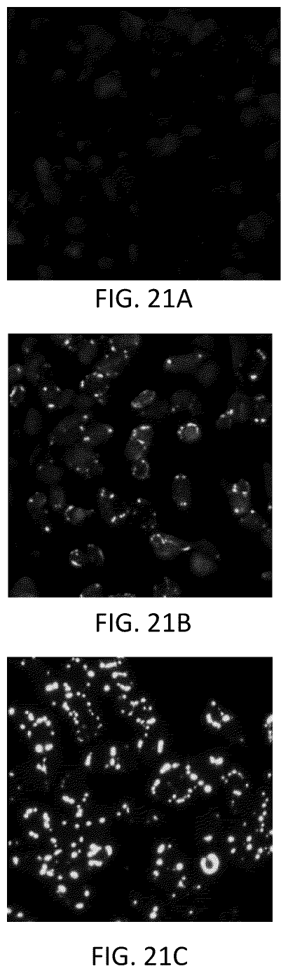

FIG. 21A-21C shows results. FIG. 21A shows the fluorescent image of control sample, which was incubated with probing strand without warhead. FIG. 21A shows low labeling background. FIG. 21B shows the fluorescent image of positive sample, which was incubated with probing strand with warhead. Small spot can be clearly observed within the cell boundary. FIG. 21C shows the fluorescent image of positive sample after amplification of the biotin signal of probing strand. The intensity of fluorescence and numbers of SGs′ spot are largely amplified by 5˜10 times.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or clement is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/-−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (c.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A kit for photoreactive labeling, comprising:
a nucleic acid anchoring strand, wherein the anchoring strand comprises a bait attachment site covalently attached to a bait molecule;
a nucleic acid probing strand, wherein the probing strand is configured to form a double-stranded structure with the anchoring strand along a complementary sequence;
a light-activated warhead disposed in the probing strand, wherein the light-activated warhead is configured to covalently bond the anchoring strand to the probing strand upon application of light energy; and
a tag bound to the probing strand, wherein the tag is a detectable label.

2. The kit of claim 1, wherein the nucleic acid in the anchoring strand and the nucleic acid in the probing strand are DNA, RNA, or both DNA and RNA.

3. The kit of claim 1, wherein the anchoring strand is longer than the probing strand, and the anchoring strand and the probing strand have at least 6 complementary nucleotides in a continuous row and at least 20 complementary nucleotides in total.

4. The kit of claim 1, wherein the double-stranded structure is at least 10 nucleotides in length.

5. The kit of claim 1, wherein a melting temperature Tm(1) of the double-stranded structure is at least 50° C.

6. The kit of claim 4, wherein the melting temperature Tm(1) of the double-stranded structure is from 52° C. to 60° C.

7. The kit of claim 1, wherein each of the anchoring strand and the probing strand are between 15 nucleotides and 40 nucleotides in length.

8. The kit of claim 1, wherein the bait molecule comprises a CLIP-tag, a HaloTag, SNAP-tag, protein A, protein G, protein L, or an RNA molecule.

9. The kit of claim 1, wherein the bait molecule is a primary antibody or a secondary antibody.

10. The kit of claim 1, wherein the tag comprises a biotin derivative, digoxigenin, a click chemistry tag, a CLIP-tag, a HaloTag, or a SNAP-tag.

11. The kit of claim 10, wherein the tag is a biotin derivative and the biotin derivative includes a moiety of

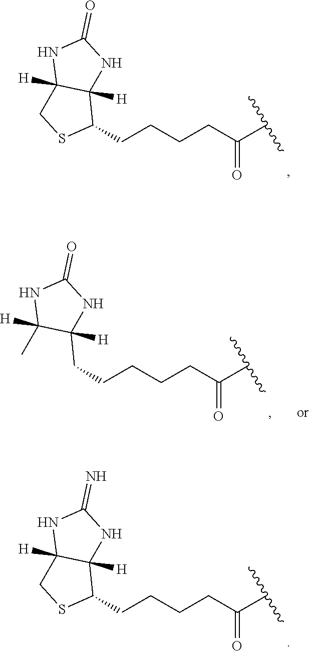

12. The kit of claim 1, wherein the light-activated warhead comprises a thymine-specific warhead.

13. The kit of claim 1, wherein the light-activated warhead is a nucleobase-specific psoralen, including a moiety of

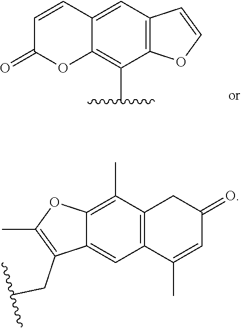 [SMILES: *c1c2occc2cc2ccc(=O)oc12 ; *Cc1c(C)oc2c(C)c3c(cc12)C(C)=CC(=O)C3]

14. The kit of claim 1, wherein the light-activated warhead is a nucleobase-specific azide.

15. The kit of claim 1, wherein the light-activated warhead is a nucleobase-specific 3-cyanovinylcarbazole nucleoside (CNVK), including a moiety of

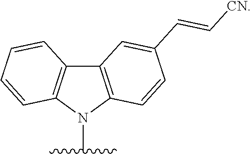 [SMILES: *n1c2ccccc2c2cc(/C=C/C#N)ccc21]

16. The kit of claim 1, wherein the light-activated warhead comprises a nucleobase-specific diazirine, including a moiety of

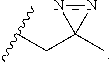 [SMILES: *CC1(C)N=N1]

17. The kit of claim 1, wherein, when the nucleic acid anchoring strand and the nucleic acid probing strand bind to each other along the complementary sequence to form the double-stranded structure, the double-stranded structure comprises a cleavable site, wherein the anchoring strand and the probing strand are configured to break at the cleavable site.

18. The kit of claim 17, wherein the cleavable site comprises a restriction enzyme site or another endonuclease site.

19. The kit of claim 17, wherein cleaving the double-stranded site at the cleavable site results in the formation of two smaller double-stranded structures, wherein a melting temperature Tm(2) of a first of the two smaller double-stranded structures is from 26° C. to 34° C., wherein the first of the smaller double-stranded structures does not contain the light-activated warhead or the tag.

20. A photoselective labeling method comprising:
delivering a nucleic acid anchoring strand to a biological sample, wherein the anchoring strand comprises a bait attachment site covalently attached to a bait molecule, and binding the bait molecule to a molecule of interest in the biological sample;
removing unbound anchoring strand from the biological sample;
delivering a nucleic acid probing strand to the biological sample, the probing strand comprising a light-activated warhead disposed therein, and a tag bound to the 3′ end of the probing strand, wherein the probing strand forms a double-stranded structure with the anchoring strand along a complementary sequence to form a photoreactive probe;
selectively illuminating a first region of the biological sample to thereby activate the light-activated warhead disposed in the probing strand and covalently bond the probing strand to the anchoring strand of the probe in the first region to achieve a photoselective labeling, and not activating the light-activated warhead in a second region of the biological sample such that the probing strand and anchoring strand are not covalently bonded in the probe in the second region;
heating the biological sample above a melting temperature Tm(1) such that the probing strand separates from the anchoring strand in probe in the second region of the biological sample to form unbound probing strand, while the probing strand and anchoring strand remain in the double-stranded structure in the first region of the biological sample; and
removing the unbound probing strand from the biological sample.

21. The photoselective labeling method of claim 19, wherein the melting temperature Tm(1) of the double-stranded structure of the photoreactive probe is from 52° C. to 60° C.


SEQUENCE LISTING (34 sequences)

SEQ ID NO: 1 - protein, 6 aa, synthetic construct
HHHHHH

SEQ ID NO: 2 - protein, 5 aa, synthetic construct
DDDDK

SEQ ID NO: 3 - protein, 6 aa, synthetic construct
LVPRGS

SEQ ID NO: 4 - protein, 8 aa, synthetic construct
LEVLFQGP

SEQ ID NO: 5 - protein, 7 aa, synthetic construct
ENLYFQS

SEQ ID NO: 6 - protein, 7 aa, synthetic construct
ENLYFQG

SEQ ID NO: 7 - protein, 6 aa, synthetic construct
LVPRGS

SEQ ID NO: 8 - protein, 13 aa, synthetic construct
GRRRYLEVLFQGP

SEQ ID NO: 9 - protein, 13 aa, synthetic construct
LEVLFQGPYRRRG

SEQ ID NO: 10 - protein, 11 aa, synthetic construct
ENLYFQGGGGS

SEQ ID NO: 11 - protein, 11 aa, synthetic construct
LVPRGSYRRRG

SEQ ID NO: 12 - protein, 6 aa, synthetic construct
LVPRGS

SEQ ID NO: 13 - protein, 13 aa, synthetic construct
LEVLFQGPGGGGS

SEQ ID NO: 14 - protein, 13 aa, synthetic construct
ENLYFQGGYRRRG

SEQ ID NO: 15 - protein, 13 aa, synthetic construct
GGGGSYENLYFQG

SEQ ID NO: 16 - protein, 40 aa, synthetic construct
  VARIANT 1..40: note = This sequence may encompass 1-8 GGGGS repeating units
GGGGSGGGGSGGGGSGGGGSGGGGSGGGGSGGGGSGGGGS

SEQ ID NO: 17 - protein, 5 aa, synthetic construct
GGGGS

SEQ ID NO: 18 - protein, 15 aa, synthetic construct
  SITE 1: note = N-acetyl Azidoalanine
  SITE 15: note = Lysine 1,2-ethylene diamine biotin
XGRRRYLEVLFQGPK

SEQ ID NO: 19 - protein, 14 aa, synthetic construct
  SITE 1: note = Lysine linked to biotin through amide functionality
  SITE 14: note = Azidolysine amide
KLEVFQGPYRRRGX

SEQ ID NO: 20 - protein, 13 aa, synthetic construct
  SITE 1: note = Lysine linked to biotin through amide functionality
  SITE 13: note = Azidolysine amide
KENLYFQGGGGSX

SEQ ID NO: 21 - protein, 13 aa, synthetic construct
  SITE 1: note = Lysine linked to biotin through amide functionality
  SITE 13: note = Azidolysine amide
KLVPRGSYRRRGX

SEQ ID NO: 22 - protein, 8 aa, synthetic construct
  SITE 1: note = Lysine linked to biotin through amide functionality
  SITE 8: note = Azidolysine amide
KLVPRGSX

SEQ ID NO: 23 - protein, 15 aa, synthetic construct
  SITE 1: note = Lysine linked to biotin through amide functionality
  SITE 15: note = Propargylglycine amide
KLEVLFQGPGGGGSX

SEQ ID NO: 24 - protein, 15 aa, synthetic construct
  SITE 1: note = Lysine linked to biotin through amide functionality
  SITE 15: note = Propargylglycine amide
KENLYFQGGYRRRGX

SEQ ID NO: 25 - protein, 15 aa, synthetic construct
  SITE 1: note = N-acetyl Azidoalanine
  SITE 15: note = Lysine 1,2-ethylene diamine biotin
XGGGGSYENLYFQGK

SEQ ID NO: 26 - protein, 13 aa, synthetic construct
  SITE 1: note = Linked to biotin through amide functionality with 6-N-acetyl benzophenone substitution
  SITE 13: note = Azidolysine amide
KENLYFQGGGGSX

SEQ ID NO: 27 - DNA, 28 bp, synthetic construct
  source 1..28: mol_type = other DNA
  misc_difference 22: note = Any nucleobase modified with a photoactive warhead
  modified_base 28: mod_base = OTHER; note = Adenosine biotin
tcttcattacgaattcatcgantcataa

SEQ ID NO: 28 - DNA, 36 bp, synthetic construct
  source 1..36: mol_type = other DNA
  modified_base 1: mod_base = OTHER; note = Antibody conjugated deoxycytidine
catcctaattatgattcgatgaattcgtaatgaaga

SEQ ID NO: 29 - DNA, 17 bp, synthetic construct
  source 1..17: mol_type = other DNA
  misc_difference 11: note = Any nucleobase modified with a photoactive warhead
  modified_base 17: mod_base = OTHER; note = Adenosine biotin
aattcatcgantcataa

SEQ ID NO: 30 - DNA, 21 bp, synthetic construct
  source 1..21: mol_type = other DNA
  modified_base 1: mod_base = OTHER; note = Antibody conjugated deoxycytidine
catcctaattatgattcgatg

SEQ ID NO: 31 - DNA, 21 bp, synthetic construct
  source 1..21: mol_type = other DNA
catcgaatcataattaggatg

SEQ ID NO: 32 - DNA, 36 bp, synthetic construct
  source 1..36: mol_type = other DNA
tcttcattacgaattcatcgaatcataattaggatg

SEQ ID NO: 33 - protein, 5 aa, synthetic construct
HHHHH

SEQ ID NO: 34 - protein, 10 aa, synthetic construct
  VARIANT 1..10: note = This sequence may encompass 7-10 residues
HHHHHHHHHH